SEQUENCE LISTING (60 sequences)

SEQ ID NO: 1 - protein, 19 aa, synthetic construct
  REGION 1..19: note = Synthetic: HCDR3 polypeptide
AKDPSYGSGSYHSYYGMDV

SEQ ID NO: 2 - protein, 9 aa, synthetic construct
  REGION 1..9: note = Synthetic: LCDR3 polypeptide
QQRFNWPLT

SEQ ID NO: 3 - protein, 16 aa, synthetic construct
  REGION 1..16: note = Synthetic: HCDR3 polypeptide
VKDFHYGSGSNYGMDV

SEQ ID NO: 4 - protein, 9 aa, synthetic construct
  REGION 1..9: note = Synthetic: LCDR3 polypeptide
QQSNDWPLT

SEQ ID NO: 5 - protein, 15 aa, synthetic construct
  REGION 1..15: note = Synthetic: HCDR3 polypeptide
TKDGSYGHFYSGLDV

SEQ ID NO: 6 - protein, 9 aa, synthetic construct
  REGION 1..9: note = Synthetic: LCDR3 polypeptide
QQRYYWPLT

SEQ ID NO: 7 - protein, 126 aa, synthetic construct
  REGION 1..126: note = Synthetic: HCVR polypeptide
EEQLVESGGDLVQPGRSLRLSCAASGFTFHDYTMHWVRQAPGKGLEWVSGISWNSGSLGYADSVKGRFTISRDNAKKSLYLQMNSLRAEDTALYYCAKDPSYGSGSYHSYYGMDVWGQGTTVTVSS

SEQ ID NO: 8 - protein, 107 aa, synthetic construct
  REGION 1..107: note = Synthetic: LCVR polypeptide
EIVLTQSPATLSLSPGERATLSCWASQSISRYLVWYQQKCGQAPRLLIYEASKRATGIPVRFSGSGSGTDFTLTISSLESEDFAVYYCQQRFNWPLTFGGGTKVEIK

SEQ ID NO: 9 - protein, 123 aa, synthetic construct
  REGION 1..123: note = Synthetic: HCVR polypeptide
EVQLAESGGDLVQSGRSLRLSCAASGITFHDYAMHWVRQPPGKGLEWVSGISWNSDYIGYADSVKGRFTISRDNAKKSLYLQMNSLRPDDTALYYCVKDFHYGSGSNYGMDVWGQGTTVTVSP

SEQ ID NO: 10 - protein, 107 aa, synthetic construct
  REGION 1..107: note = Synthetic: LCVR polypeptide
EIVMTQSPATLSMSPGERATLSCRASQSVSRNLAWYQQKVGQAPRLLISGASTRATGIPARFSGSGSGTEFTLTINSLQSEDFAVYYCQQSNDWPLTFGQGTRLEIK

SEQ ID NO: 11 - protein, 122 aa, synthetic construct
  REGION 1..122: note = Synthetic: HCVR polypeptide
EVQLVESGGGLVQPGRSLRLSCAASGFTFYDYAMHWVRQAPGKGLEWVSGISWNSDTIGYADSVKGRFTISRDNAKNSLYLQMNSLRAEDTALYYCTKDGSYGHFYSGLDVWGQGTTVTVSS

SEQ ID NO: 12 - protein, 107 aa, synthetic construct
  REGION 1..107: note = Synthetic: LCVR polypeptide
EIVLTQSPATLSLSPGERATLSCRASQSVSSYLAWYQQKPGQAPRLLIYVASNRATGIPARFSGSGSGTDFTLTISSLEPDDFAVYYCQQRYYWPLTFGGGTKVEIK

SEQ ID NO: 13 - protein, 122 aa, synthetic construct
  REGION 1..122: note = Synthetic: HCVR polypeptide
EVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSS

SEQ ID NO: 14 - protein, 107 aa, synthetic construct
  REGION 1..107: note = Synthetic: LCVR polypeptide
EIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIK

SEQ ID NO: 15 - protein, 8 aa, synthetic construct
  REGION 1..8: note = Synthetic: HCDR1 polypeptide
GFTFYDYA

SEQ ID NO: 16 - protein, 8 aa, synthetic construct
  REGION 1..8: note = Synthetic: HCDR2 polypeptide
ISWNSGYI

SEQ ID NO: 17 - protein, 15 aa, synthetic construct
  REGION 1..15: note = Synthetic: HCDR3 polypeptide
AKDNSYGKFYYGLDV

SEQ ID NO: 18 - protein, 6 aa, synthetic construct
  REGION 1..6: note = Synthetic: LCDR1 polypeptide
QSVSSN

SEQ ID NO: 19 - skipped

SEQ ID NO: 20 - protein, 9 aa, synthetic construct
  REGION 1..9: note = Synthetic: LCDR3 polypeptide
QQYNNWPIT

SEQ ID NO: 21 - protein, 39 aa, synthetic construct
  REGION 1..39: note = Synthetic: CD8-alpha hinge domain polypeptide
PTPAPTIASQPLSLRPEACRPAAGGAVHTRGLDFACDIY

SEQ ID NO: 22 - protein, 47 aa, synthetic construct
  REGION 1..47: note = Synthetic: CD8-alpha hinge domain polypeptide
TTTPAPRPPTPAPTIASQPLSLRPEACRPAAGGAVHTRGLDFACDIY

SEQ ID NO: 23 - protein, 22 aa, synthetic construct
  REGION 1..22: note = Synthetic: CD8-alpha transmembrane domain polypeptide
IWAPLAGTCGVLLLSLVITLYC

SEQ ID NO: 24 - protein, 112 aa, synthetic construct
  REGION 1..112: note = Synthetic: CD3z signaling domain polypeptide
RVKFSRSADAPAYQQGQNQLYNELNLGRREEYDVLDKRRGRDPEMGGKPQRRKNPQEGLYNELQKDKMAEAYSEIGMKGERRRGKGDGLYQGLSTATKDTYDALHMQALPPR

SEQ ID NO: 25 - protein, 112 aa, synthetic construct
  REGION 1..112: note = Synthetic: CD3z signaling domain polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 42 aa, synthetic construct
  REGION 1..42: note = Synthetic: 4-1BB costimulatory domain polypeptide
KRGRKKLLYIFKQPFMRPVQTTQEEDGCSCRFPEEEEGGCEL

SEQ ID NO: 27 - protein, 48 aa, synthetic construct
  REGION 1..48: note = Synthetic: CD27 costimulatory domain polypeptide
QRRKYRSNKGESPVEPAEPCHYSCPREEEGSTIPIQEDYRKPEPACSP

SEQ ID NO: 28 - protein, 21 aa, synthetic construct
  REGION 1..21: note = Synthetic: secretion signal
MALPVTALLLPLALLLHAARP

SEQ ID NO: 29 - protein, 114 aa, synthetic construct
  REGION 1..114: note = Synthetic: IL-15 polypeptide
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 30 - protein, 23 aa, synthetic construct
  REGION 1..23: note = Synthetic: P2A cleavage sequence polypeptide
SGSGATNFSLLKQAGDVEENPGP

SEQ ID NO: 31 - protein, 4 aa, synthetic construct
  REGION 1..4: note = Synthetic: furin cleavage sequence polypeptide
RAKR

SEQ ID NO: 32 - protein, 27 aa, synthetic construct
  REGION 1..27: note = Synthetic: P2A+furin cleavage sequence polypeptide
RAKRSGSGATNFSLLKQAGDVEENPGP

SEQ ID NO: 33 - protein, 19 aa, synthetic construct
  REGION 1..19: note = Synthetic: P2A cleavage sequence polypeptide
ATNFSLLKQAGDVEENPGP

SEQ ID NO: 34 - protein, 23 aa, synthetic construct
  REGION 1..23: note = Synthetic: F2A cleavage sequence polypeptide
VKQTLNNFDLLKLAGDVESNPGP

SEQ ID NO: 35 - protein, 20 aa, synthetic construct
  REGION 1..20: note = Synthetic: E2A cleavage sequence polypeptide
QCTNYALLKLAGDVESNPGP

SEQ ID NO: 36 - protein, 17 aa, synthetic construct
  REGION 1..17: note = Synthetic: T2A cleavage sequence polypeptide
EGRSLLTCGDVEENPGP

SEQ ID NO: 37 - DNA, 553 bp, synthetic construct
  misc_feature 1..553: note = Synthetic: IRES
  source 1..553: mol_type = other DNA
ctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgata

SEQ ID NO: 38 - DNA, 461 bp, synthetic construct
  misc_feature 1..461: note = Synthetic: IRES
  source 1..461: mol_type = other DNA
agcaggtttccccaactgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccac

SEQ ID NO: 39 - protein, 493 aa, synthetic construct
  REGION 1..493: note = Synthetic: 3H7 CD8 CD27z polypeptide
MSVPTQVLGLLLLWLTDARCEIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIKGGGGSGGGGSGGGGEVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSSTTTPAPRPPTPAPTIASQPLSLRPEACRPAAGGAVHTRGLDFACDIYIWAPLAGTCGVLLLSLVITLYCQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 40 - protein, 492 aa, synthetic construct
  REGION 1..492: note = Synthetic: 3B9-CD8-BBz polypeptide
MSVPTQVLGLLLLWLTDARCEIVMTQSPATLSVSPGERATLSCRASQSVSSNLAWYQQKPGQAPRLLIYGTSTRATGIPARFSGSGSGTEFTLTISSLQSEDFAVYYCQQYNNWPLTFGGGTKVEIKGGGGSGGGGSGGGGEVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 41 - protein, 487 aa, synthetic construct
  REGION 1..487: note = Synthetic: 3H7-CD8-BBz polypeptide
MSVPTQVLGLLLLWLTDARCEIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIKGGGGSGGGGSGGGGEVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 42 - protein, 488 aa, synthetic construct
  REGION 1..488: note = Synthetic: 2B7-CD8-BBz polypeptide
MSVPTQVLGLLLLWLTDARCEIVLTQSPATLSLSPGERAALSCRASQSVSNYLAWYQQKPGQAPRLLIYDASNRATGIPARFSGSGSGTDFTLTISSLEPEDFAVYYCQQRSNWPLTFGGGTKVEIRGGGGSGGGGSGGGGEVQLVESGGGLVQPGRSLRLSCAASGFTFRDYTMHWVRQGPGKGLEWVSGISWNSDYIGYADSVKGRFTISRDNAKNSLYLQMNSLRVEDTALYYCAKLSGTYRDYFYGVDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 43 - protein, 486 aa, synthetic construct
  REGION 1..486: note = Synthetic: 9C11-CD8-BBz polypeptide
MSVPTQVLGLLLLWLTDARCEIVVTQSPATLSLSPGERATLSCRTSQTTTSYLAWYRQKPGQAPRLLIYDASNRAAGIPARFSGSGSGTDFTLTINSLEPEDFAVYYCQLRTNWITFGQGTRLEIKGGGGSGGGGSGGGGQVQLVESGGDSVKPGGSLRLSCAASGFTFSDSYMTWIRQAPGKGLEWVSFISSSGSTIYYADSVKGRFTISRDNVKKSLYLQMNRLRAEDTAVYYCAREEPGNYVYYGMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 44 - protein, 445 aa, synthetic construct
  REGION 1..445: note = Synthetic: 3H7-CD3z polypeptide
MSVPTQVLGLLLLWLTDARCEIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIKGGGGSGGGGSGGGGEVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSSTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 45 - DNA, 1482 bp, synthetic construct
  misc_feature 1..1482: note = Synthetic: 3H7-CD8-27z nucleotide
  source 1..1482: mol_type = other DNA
atgtccgtgcctacccaggtgctgggcctgctgctgctgtggctgaccgacgccagatgcgaaatagtgatgacgcagtctccagccaccctgtctgtgtctccaggggaaagaaccaccctctcctgcagggccagtcagagtgttagcagcaacttagcctggtaccttcagaaacctggccaggctcccaggctcctcatctatggtgcatccaccagggccactggtatcccagccaggttcagtggcagtgggtctgggacagagttcattctcaccatcagcagcctgcagtctgaagattttgcagtttattactgtcagcagtataataactggccgatcaccttcggccaagggacacggctggagattaaaggtggaggtggatctggaggaggaggatccggtggaggaggtgaagtgcaactggtggagtctgggggaggcttggtacagcctggcaggtccctgagactctcctgtgcagcctctggattcaccttttatgattatgccatgcactgggtccggcaagctccagggaagggcctggagtgggtctcaggtattagttggaatagtggttacataggctatgcggactctgtgaagggccgattcaccatctccagagacaacgccaagaactccctgtatctgcaaatgaacagtctgagagctgaggacacggccttgtattactgtgcaaaagataacagctatggaaagttctactacggtttggacgtctggggccaagggaccacggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccaacgacgcaagtaccgctccaataaaggagagtcaccagtagaacccgccgaaccttgtcactattcatgtccacgcgaagaggagggttcaacgatccctattcaggaagattacagaaagccggaacctgcttgtagccccagagtgaagttcagccgcagcgccgacgcccctgcctaccagcagggccagaaccagctgtataacgagctgaacctgggcaggcgggaggaatacgacgtgctggacaagcgcagaggccgggaccctgagatgggcggcaagccccagaggcggaagaacccccaggaaggcctgtataacgaactgcagaaagacaagatggccgaggcctacagcgagatcggcatgaagggcgagcggcgacgcggcaagggccacgacggcctgtaccagggcctgtccaccgccaccaaggacacctacgacgccctgcacatgcaggccctgcctccccgttag

SEQ ID NO: 46 - DNA, 1464 bp, synthetic construct
  misc_feature 1..1464: note = Synthetic: 3H7-CD8-BBz nucleotide
  source 1..1464: mol_type = other DNA
atgtccgtgcctacccaggtgctgggcctgctgctgctgtggctgaccgacgccagatgcgaaatagtgatgacgcagtctccagccaccctgtctgtgtctccaggggaaagaaccaccctctcctgcagggccagtcagagtgttagcagcaacttagcctggtaccttcagaaacctggccaggctcccaggctcctcatctatggtgcatccaccagggccactggtatcccagccaggttcagtggcagtgggtctgggacagagttcattctcaccatcagcagcctgcagtctgaagattttgcagtttattactgtcagcagtataataactggccgatcaccttcggccaagggacacggctggagattaaaggtggaggtggatctggaggaggaggatccggtggaggaggtgaagtgcaactggtggagtctgggggaggcttggtacagcctggcaggtccctgagactctcctgtgcagcctctggattcaccttttatgattatgccatgcactgggtccggcaagctccagggaagggcctggagtgggtctcaggtattagttggaatagtggttacataggctatgcggactctgtgaagggccgattcaccatctccagagacaacgccaagaactccctgtatctgcaaatgaacagtctgagagctgaggacacggccttgtattactgtgcaaaagataacagctatggaaagttctactacggtttggacgtctggggccaagggaccacggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 47 - DNA, 1479 bp, synthetic construct
  misc_feature 1..1479: note = Synthetic: 3B9-CD8-BBz nucleotide
  source 1..1479: mol_type = other DNA
atgagcgttccaacccaagttctgggactgcttctgctctggttgactgacgctaggtgcgaaatagtaatgacccaatccccagccactctctccgttagcccaggtgaaagagccactcttagttgcagggctagtcaatccgtatctagcaacctggcctggtaccagcaaaagcccggacaagcgccgcggttgttgatctatgggacgagcacacgagctacgggtattccggccaggttctcagggtctggctccggaaccgaatttacattgacgatcagtagtctgcaatcagaggatttcgccgtttactattgccaacagtacaataattggccgctcacattcgggggaggaaccaaggtcgagattaagggaggtgggggtagtgggggcggggggtcaggaggtggaggagaggtacagttggtagaaagcggcggggggttggttcaacctggacggagtctgagattgtcttgcgtggcttccggctttactttcaatgattacgccatgcactgggtacgccaggcgcctggaaagggtctggagtgggtttccgtgatatcctggaatagtgatagtataggctatgccgatagtgtaaaaggaaggtttacaatctctagggataacgctaagaacagcctgtaccttcaaatgcatagtctccgggctgaggacacagccttgtactattgtgctaaggacaatcattatggaagcgggtcatattattactatcaatatgggatggatgtgtggggtcagggaacgaccgttacggtatcctcaaccaccacccctgcaccaaggcccccgactcccgcgcccaccatcgcgtcacagcctcttagcctgcgaccggaagcatgcagaccagctgccgggggggccgtgcatacgagaggtttggacttcgcctgcgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 48 - DNA, 1467 bp, synthetic construct
  misc_feature 1..1467: note = Synthetic: 2B7-CD8-BBz nucleotide
  source 1..1467: mol_type = other DNA
atgtccgtacctacccaggtgctgggcctgctgctgctgtggctgaccgacgccagatgcgaaattgtgttgacacagtctccagccaccctgtctttgtctccaggggaaagagccgccctctcctgcagggccagtcagagtgttagcaactacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgctcactttcggcggagggaccaaggtggagatcagaggtggaggtggatctggaggaggaggatccggtggaggaggtgaagtgcagctggtggagtctgggggaggcttggtacagcctggcaggtccctgcgactctcctgtgcagcctctggattcacctttcgagattataccatgcactgggtccggcaaggtccagggaagggcctggaatgggtctcaggtattagttggaatagtgattacataggctatgcggactctgtgaagggccgattcaccatctccagagacaacgccaagaactccctgtatctgcaaatgaacagtctgagagttgaggacacggccttgtattactgtgcaaagctcagtgggacctacagggactacttctacggagtggacgtctggggccaagggaccacggtcaccgtctcctcaaccaccacccctgcaccaaggcccccgactcccgcgcccaccatcgcgtcacagcctcttagcctgcgaccggaagcatgcagaccagctgccgggggggccgtgcatacgagaggtttggacttcgcctgcgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 49 - DNA, 1461 bp, synthetic construct
  misc_feature 1..1461: note = Synthetic: 9C11-CD8-BBz nucleotide
  source 1..1461: mol_type = other DNA
atgtccgtgcctacccaggtgctgggcctgctgctgctgtggctgaccgacgccagatgcgaaattgtggtgacacagtctccagccaccctgtctttgtctccaggggaaagagccaccctctcctgcaggaccagtcagactactaccagctacttagcctggtaccgacagaaacctggccaggctcccaggctcctcatctatgatgcatccaacagggccgctggcatcccagccaggttcagtggcagtgggtctgggacagacttcactctcaccatcaacagcctggagcctgaagattttgcagtttattactgtcagctgcgtaccaactggatcaccttcggccaagggacacgactggagattaaaggtggaggtggatctggaggaggaggatccggtggaggaggtcaggtgcagctggtggagtctgggggagactcggtcaagcctggagggtccctgagactctcctgtgcagcctctggattcaccttcagtgactcctacatgacttggatccgccaggctccagggaaggggctggagtgggtttcattcattagtagtagtggaagtaccatatattatgcagactctgtgaagggccgattcaccatttccagggacaacgtcaagaagtcattgtatctgcagatgaacagactgagagccgaggacacggccgtgtattactgtgcgagagaagaaccaggaaactacgtctattacggtatggacgtctggggccaagggaccacggtcaccgtctcctcaaccaccacccctgcaccaaggcccccgactcccgcgcccaccatcgcgtcacagcctcttagcctgcgaccggaagcatgcagaccagctgccgggggggccgtgcatacgagaggtttggacttcgcctgcgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 50 - DNA, 135 bp, synthetic construct
  misc_feature 1..135: note = Synthetic: Codon-optimized CD8-alpha hinge region nucleotide
  source 1..135: mol_type = other DNA
accaccacccctgcaccaaggcccccgactcccgcgcccaccatcgcgtcacagcctcttagcctgcgaccggaagcatgcagaccagctgccgggggggccgtgcatacgagaggtttggacttcgcctgcgat

SEQ ID NO: 51 - DNA, 135 bp, synthetic construct
  misc_feature 1..135: note = Synthetic: Codon-optimized CD8-alpha hinge region nucleotide
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 52 - protein, 644 aa, synthetic construct
  REGION 1..644: note = Synthetic: anti-CD20-CAR polypeptide
MSVPTQVLGLLLLWLTDARCEIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIKGGGGSGGGGSGGGGEVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 53 - protein, 22 aa, synthetic construct
  REGION 1..22: note = Synthetic: P2A cleavage domain polypeptide
GSGATNFSLLKQAGDVEENPGP

SEQ ID NO: 54 - protein, 671 aa, synthetic construct
  REGION 1..671: note = Synthetic: anti-CD20-CAR polypeptide
MSVPTQVLGLLLLWLTDARCEIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIKGGGGSGGGGSGGGGEVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 55 - protein, 48 aa, synthetic construct
  REGION 1..48: note = Synthetic: Secretion signal polypeptide
MRISKPHLRSISIQCYLCLLLNSHFLTEAGIHVFILGCFSAGLPKTEA

SEQ ID NO: 56 - DNA, 1935 bp, synthetic construct
  misc_feature 1..1935: note = Synthetic: anti-CD20 CAR + sIL15 nucleotide
  source 1..1935: mol_type = other DNA
atgtccgtgcctacccaggtgctgggcctgctgctgctgtggctgaccgacgccagatgcgaaatagtgatgacgcagtctccagccaccctgtctgtgtctccaggggaaagaaccaccctctcctgcagggccagtcagagtgttagcagcaacttagcctggtaccttcagaaacctggccaggctcccaggctcctcatctatggtgcatccaccagggccactggtatcccagccaggttcagtggcagtgggtctgggacagagttcattctcaccatcagcagcctgcagtctgaagattttgcagtttattactgtcagcagtataataactggccgatcaccttcggccaagggacacggctggagattaaaggtggaggtggatctggaggaggaggatccggtggaggaggtgaagtgcaactggtggagtctgggggaggcttggtacagcctggcaggtccctgagactctcctgtgcagcctctggattcaccttttatgattatgccatgcactgggtccggcaagctccagggaagggcctggagtgggtctcaggtattagttggaatagtggttacataggctatgcggactctgtgaagggccgattcaccatctccagagacaacgccaagaactccctgtatctgcaaatgaacagtctgagagctgaggacacggccttgtattactgtgcaaaagataacagctatggaaagttctactacggtttggacgtctggggccaagggaccacggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtagcggggctacgaacttctcccttcttaaacaagcgggagacgtggaagaaaatcccggacctatggccttaccagtgaccgccttgctcctgccgctggccttgctgctccacgccgccaggccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 57 - DNA, 2016 bp, synthetic construct
  misc_feature 1..2016: note = Synthetic: anti-CD20 CAR + sIL15 nucleotide
  source 1..2016: mol_type = other DNA
atgtccgtgcctacccaggtgctgggcctgctgctgctgtggctgaccgacgccagatgcgaaatagtgatgacgcagtctccagccaccctgtctgtgtctccaggggaaagaaccaccctctcctgcagggccagtcagagtgttagcagcaacttagcctggtaccttcagaaacctggccaggctcccaggctcctcatctatggtgcatccaccagggccactggtatcccagccaggttcagtggcagtgggtctgggacagagttcattctcaccatcagcagcctgcagtctgaagattttgcagtttattactgtcagcagtataataactggccgatcaccttcggccaagggacacggctggagattaaaggtggaggtggatctggaggaggaggatccggtggaggaggtgaagtgcaactggtggagtctgggggaggcttggtacagcctggcaggtccctgagactctcctgtgcagcctctggattcaccttttatgattatgccatgcactgggtccggcaagctccagggaagggcctggagtgggtctcaggtattagttggaatagtggttacataggctatgcggactctgtgaagggccgattcaccatctccagagacaacgccaagaactccctgtatctgcaaatgaacagtctgagagctgaggacacggccttgtattactgtgcaaaagataacagctatggaaagttctactacggtttggacgtctggggccaagggaccacggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtagcggggctacgaacttctcccttcttaaacaagcgggagacgtggaagaaaatcccggacctatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 58 - protein, 487 aa, synthetic construct; identical to SEQ ID NO: 41
  REGION 1..487: note = Synthetic: anti-CD20-CAR polypeptide

SEQ ID NO: 59 - protein, 135 aa, synthetic construct
  REGION 1..135: note = Synthetic: Secretion signal and sIL-15 domain polypeptide
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 60 - DNA, 2489 bp, synthetic construct
  misc_feature 1..2489: note = Synthetic: anti-CD20 CAR + sIL15 nucleotide
  source 1..2489: mol_type = other DNA
atgtccgtgcctacccaggtgctgggcctgctgctgctgtggctgaccgacgccagatgcgaaatagtgatgacgcagtctccagccaccctgtctgtgtctccaggggaaagaaccaccctctcctgcagggccagtcagagtgttagcagcaacttagcctggtaccttcagaaacctggccaggctcccaggctcctcatctatggtgcatccaccagggccactggtatcccagccaggttcagtggcagtgggtctgggacagagttcattctcaccatcagcagcctgcagtctgaagattttgcagtttattactgtcagcagtataataactggccgatcaccttcggccaagggacacggctggagattaaaggtggaggtggatctggaggaggaggatccggtggaggaggtgaagtgcaactggtggagtctgggggaggcttggtacagcctggcaggtccctgagactctcctgtgcagcctctggattcaccttttatgattatgccatgcactgggtccggcaagctccagggaagggcctggagtgggtctcaggtattagttggaatagtggttacataggctatgcggactctgtgaagggccgattcaccatctccagagacaacgccaagaactccctgtatctgcaaatgaacagtctgagagctgaggacacggccttgtattactgtgcaaaagataacagctatggaaagttctactacggtttggacgtctggggccaagggaccacggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagagtactgcggccgctacgtaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgatattaattaagccaccgccatggccttaccagtgaccgccttgctcctgccgctggccttgctgctccacgccgccaggccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

(19) United States
(12) Patent Application Publication
(10) Pub. No.: US 2025/0025504 A1
(43) Pub. Date: Jan. 23, 2025

(54) TREATMENT OF B CELL MALIGNANCIES

(71) Applicant: ADICET THERAPEUTICS, INC., Redwood City, CA (US)

(72) Inventors: Blake AFTAB, Thousand Oaks, CA (US); Francesco GALMI, Del Mar, CA (US); Rose Kamyee LAI, San Ramon, CA (US); Ori MALLER, Half Moon Bay, CA (US)

(21) Appl. No.: 18/716,497

(22) PCT Filed: Dec. 5, 2022
(86) PCT No.: PCT/US22/51848

§ 371 Date: Jun. 4, 2024

Related U.S. Application Data
(60) Provisional application No. 63/286,086, filed on Dec. 5, 2021.

(51) Int. Cl. A61K 35/17; A61K 31/675; A61K 31/7076; A61K 39/00; A61P 35/00; C07K 14/725; C07K 16/28

(52) U.S. Cl. CPC A61K 35/17; A61K 31/675; A61K 31/7076; A61K 39/4611; A61K 39/4631; A61K 39/464424; A61P 35/00; C07K 14/7051; C07K 16/2887

(57) ABSTRACT

Aspects of the disclosure include methods of effectively treating a relapsed/refractory B cell malignancy in a patient in need thereof who has previously been treated with and ultimately failed at least one, two, or at least three previous therapies. In one example. a method comprises administering to the subject one or more doses comprising a therapeutically effective amount of anti-CD20 CAR γδ T cells that express a chimeric antigen receptor (CAR) comprising a binding domain that specifically binds to CD20 on a malignant B cell to the subject. Following administration, the subject methods optionally further include monitoring for one or more biomarkers of cell activation and/or therapeutic efficacy that inform the need for and/or type of follow-on treatment regimen.

### CROSS-REFERENCES TO RELATED APPLICATIONS

This application claims the benefit of priority to U.S. Provisional Application No. 63/286,086, filed Dec. 5, 2021, the contents of which are hereby incorporated for any and all purposes.

### FIELD OF DISCLOSURE

The present disclosure relates generally to a remarkably effective salvage therapy for relapsed/refractory B cell malignancies, comprising administering one or more doses of anti-CD20 CAR γδ T cells to a patient previously treated with one or more alternative therapies.

### BACKGROUND OF THE DISCLOSURE

Non-Hodgkin Lymphoma (NHL) is a type of cancer that affects the lymphatic system. NHL is not a single disease, but rather comprises a number of closely related cancers. Although the various types of NHL share many common characteristics, they differ in certain features, including their appearance under a microscope, molecular features and growth patterns, impact on the body, and how they respond to different types of treatment. NHL is estimated to be the thirteenth most common cancer and the eleventh leading cause of cancer death worldwide (Bray et al. (2018) CA Cancer J Clin., 68 (6): 394-424; Global Cancer Observatory: Cancer Tomorrow. International Agency for Research on Cancer).

Subtypes of NHL are classified by which lymphocyte subset is transformed and by how quickly the lymphoma grows and progresses. NHL arises in B cells, T cells, and natural killer (NK) cells, but the vast majority (85-90%) of NHL subtypes originate in B cells. NHL subtypes are designated as either indolent (slow-growing) or aggressive (fast-growing), and about 60% of all cases of NHL are of an aggressive subtype. Relevant examples of subtypes of aggressive B cell lymphomas include diffuse large B cell lymphoma (DLBCL), primary mediastinal large B cell lymphoma (PMBCL), mantle cell lymphoma (MCL), and transformed follicular lymphoma (tFL).

The anti-CD20 monoclonal antibody (mAb) RITUXAN® (rituximab) combined with chemotherapy is a first-line induction therapy of CD20 positive B cell NHLs. Rituximab engages Fc receptors on NK cells and macrophages, facilitates complement-dependent cytotoxicity (CDC) and antibody-dependent cell-mediated cytotoxicity/phagocytosis (ADCC/ADCP), and exerts direct antiproliferative and pro-apoptotic effects (Tobinai et al. (2017) Adv Ther., 34(2): 324-356; Boross and Leusen (2012) Am J Cancer Res., 2(6): 676-690). While the use of rituximab has significantly improved outcomes in some patients with B-cell malignancies (Lim and Levy (2014) J Immunol., 193(4): 1519-1524; Coiffier et al. (2010) Blood, 116(12): 2040-2045; Pfreundschuh et al. (2006) Lancet Oncol., 7(5): 379-391; Hallek et al. (2010) Lancet, 376(9747): 1164-1174), resistance towards rituximab is observed in about half of patients during a course of prolonged treatment. The precise mechanism of resistance to rituximab is unknown, but it is thought to be a function of a complex combination of the three mechanisms of action of rituximab (CDC, ADCC/ADCP, and apoptosis), as well as the patient-specific lymphoma microenvironment (van Meetern and Hagenbeek (2009) The Netherlands Journal of Medicine, 67(7): 251-259). Although alternative anti-CD20 mAbs have been developed, their efficacy and safety compared to rituximab are still controversial (Luo et al. (2021) Scientific Reports, 11(3255)).

Adoptive cell therapy, specifically autologous CAR αβ T cell therapy, has played an increasingly relevant role as a salvage treatment for relapsed/refractory B cell NHLs, including in particular the anti-CD19 αβ CAR T cell therapy Yescarta™ (axicabtagene ciloleucel), which is approved for the treatment of adult patients with R/R DLBCL. Unfortunately, however, 30% of patients have only a partial response to the treatment, and the therapeutic effects tend to wane within six months in many others, with only 40% of patients achieving durable responses. (With FDA Approval for Advanced Lymphoma, Second CAR T-Cell Therapy Moves to the Clinic; (2017)). Moreover, many eligible patients do not have access to the cell product due to manufacturing failures and/or cell infusion delays, and these cellular therapies can also present their own difficult toxicities, namely graft-versus-host disease (GvHD) and immunogenicity (i.e., host-versus graft rejection), as well as cytokine release syndrome (CRS). Accordingly, while autologous CAR αβ T cell therapies have positively impacted the treatment paradigm for B cell NHL, a myriad of issues remain, including but not limited to challenges in manufacturability, non-responders, serious adverse events, and relapse after achieving remission.

As such, the outcome of current salvage therapies remains poor for many relapsed or refractory (R/R) B-cell non-Hodgkin lymphoma (NHL) patients, and more effective treatment options are urgently needed, and for heavily pre-treated patients in particular.

### SUMMARY OF DISCLOSURE

The present disclosure addresses the urgent unmet need in the art for more effective salvage therapies for patients suffering from relapsed or refractory (R/R) B-cell malignancies, by administering at least one dose of anti-CD20 CAR γδ T cells to patients who have been previously treated with at least one, at least two, or at least three, previous therapies. As demonstrated herein for the first time, the subject γδ T cell therapy provides unexpected and truly remarkable results in patients who have failed multiple prior treatments, including previous anti-CD20 antibody and/or autologous αβ CAR T cell therapies.

The subject methods will find advantageous use in a wide range of B cell malignancies including, e.g., Non-Hodgkin lymphoma (NHL); chronic lymphocytic leukemia/small lymphocytic lymphoma (CLL/SLL), acute lymphocytic leukemia (ALL), and acute myeloid leukemia (AML). In embodiments, the B cell malignancy may be a type of NHL including diffuse large B cell lymphoma (DLBCL), mantle cell lymphoma (MCL), transformed follicular lymphoma (tFL), primary mediastinal (thymic) large B cell lymphoma (PMBCL), high-grade B cell lymphomas, Burkitt lymphoma, follicular lymphoma (FL), and marginal zone lymphoma (MZL).

In one aspect, the invention provides methods of treating a relapsed/refractory (R/R) B cell malignancy in a patient who has been previously treated with at least one, at least two, or at least three, previous therapies, comprising administering to the subject a therapeutically effective amount of anti-CD20 CAR γδ T cells that express a chimeric antigen receptor (CAR) comprising a binding domain that specifically binds to CD20 on a malignant B cell, and thereby treating the subject. In embodiments, the patient may be relapsed from or refractory to a previous therapy comprising an anti-CD20 monoclonal antibody (e.g. rituximab or obinutuzumab) or an anti-CD19 CAR αβ T cell therapy (e.g. axicabtagene ciloleucel or tisagenlecleucel).

In embodiments, the binding domain of the anti-CD20 CAR γδ T cells specifically binds to a CD20 epitope that is different from the CD20 epitope recognized by the anti-CD20 monoclonal antibody.

In embodiments, the therapeutically effective amount of said anti-CD20 CAR γδ T cells is between about 3×10⁷ and about 1×10⁹ γδ T cells. In embodiments, the therapeutically effective amount of said anti-CD20 CAR γδ T cells is about 3×10⁷ γδ T cells. In embodiments, the therapeutically effective amount of said anti-CD20 CAR γδ T cells is about 1×10⁸ γδ T cells. In embodiments, the therapeutically effective amount of said anti-CD20 CAR γδ T cells is about 3×10⁸ γδ T cells. In embodiments, the therapeutically effective amount of said anti-CD20 CAR γδ T cells is about 1×10⁹ γδ T cells. The anti-CD20 CAR γδ T cells may comprise, consist essentially of or consist of γδ1 T cells, γδ2 T cells, γδ3 T cells, or γδ4 T cells, or combinations thereof. In exemplary embodiments anti-CD20 CAR γδ T cells comprise γδ1 T cells.

In embodiments, the methods may further comprise administering to the subject a lymphodepletion (LD) regimen prior to administering to the subject a first dose of the therapeutically effective amount of anti-CD20 CAR γδ T cells. In some embodiments, the LD regimen comprises administration of fludarabine at about 30 mg/m²/day for three days plus cyclophosphamide at about 500 mg/m²/day for three days, optionally further comprising an anti-CD52 antibody and/or an anti-CD19 antibody. In some embodiments, the LD regimen comprises administration of fludarabine at about 30 mg/m²/day for four days plus cyclophosphamide at about 1000 mg/m²/day for three days, optionally further comprising an anti-CD52 antibody and/or an anti-CD19 antibody.

In embodiments, the subject methods further comprise administering one or more additional doses of anti-CD20 CAR γδ T cells at least 5 days, at least 7 days, at least 10 days, at least 14 days, at least 21 days, at least 28 days, or at least one month after the first dose. In embodiments, the one or more additional doses of anti-CD20 CAR γδ T cells are administered without an additional LD regimen. In embodiments, the one or more additional doses of anti-CD20 CAR γδ T cells are administered following an additional LD regimen. In embodiments, the one or more additional doses comprise an increased amount of anti-CD20 CAR γδ T cells. In embodiments, the one or more additional doses comprise a decreased amount of anti-CD20 CAR γδ T cells. In embodiments, the one or more additional doses comprise the same amount of anti-CD20 CAR γδ T cells. In embodiments, the one or more additional doses comprise anti-CD20 CAR γδ T cells derived from the same donor. In embodiments, the one or more additional doses comprise anti-CD20 CAR γδ T cells derived from a different donor.

In an exemplary embodiment, the methods comprise administering a LD regimen to the subject on Day −5, followed by administering a first dose comprising a therapeutically effective amount of anti-CD20 CAR γδ T cells (e.g. between about 3×10⁷ and about 1×10^{9 γδ} T cells) to the subject on Day 1. In embodiments, the therapeutically effective amount of said anti-CD20 CAR γδ T cells is about 3×10⁷ γδ T cells. In embodiments, the therapeutically effective amount of said anti-CD20 CAR γδ T cells is about 1×10⁸ γδ T cells. In embodiments, the therapeutically effective amount of said anti-CD20 CAR γδ T cells is about 3×10⁸ γδ T cells. In embodiments, the therapeutically effective amount of said anti-CD20 CAR γδ T cells is about 1×10⁹ γδ T cells. The anti-CD20 CAR γδ T cells may comprise, consist essentially of or consist of γδ1 T cells, γδ 2 T cells, γδ3 T cells, or γδ4 T cells, or combinations thereof. In preferred embodiments the anti-CD20 CAR γδ T cells comprise, consist essentially of, or consist of γδ1 T cells.

In further exemplary embodiments, the subject methods may optionally further comprise administering a second dose comprising a therapeutically effective amount of anti-CD20 CAR γδ T cells to the subject on Day 7, with or without administering an additional LD regimen. The second therapeutically effective dose may comprise an increased amount of anti-CD20 CAR γδ T cells, a decreased amount of anti-CD20 CAR γδ T cells, or the same amount of anti-CD20 CAR γδ T cells as the first dose. In embodiments, the subject methods may optionally further comprise administering a third dose comprising a therapeutically effective amount of anti-CD20 CAR γδ T cells to the subject on Day 14, with or without administering an additional LD regimen. The third therapeutically dose may comprise an increased amount of anti-CD20 CAR γδ T cells, a decreased amount of anti-CD20 CAR γδ T cells, or the same amount of anti-CD20 CAR γδ T cells as the first and/or second doses. In embodiments, the therapeutically effective amount of said anti-CD20 CAR γδ T cells is about 3×10⁸ γδ T cells. The anti-CD20 CAR γδ T cells may comprise, consist essentially of or consist of γδ1 T cells, γδ2 T cells, γδ3 T cells, or γδ4 T cells, or combinations thereof. In preferred embodiments the anti-CD20 CAR γδ T cells comprise, consist essentially of, or consist of γδ1 T cells.

In embodiments, the subject methods may further comprise administering a therapeutically effective amount of IL-2 to the subject. In some embodiments, the therapeutically effective amount of IL-2 is about 2×10⁶ IU daily from 1-14 days following administration of the anti-CD20 CAR γδ T cells.

In another aspect, the methods may further include monitoring the patient for one or more pharmacodynamics/pharmacokinetics biomarkers, as an indication of cell product activation and/or efficacy. In embodiments, the subject methods further comprise monitoring the subject for one or more pharmacodynamics/pharmacokinetics biomarkers following administration of the anti-CD20 CAR γδ T cells, wherein the biomarkers are selected from the group comprising or consisting of CAR transgene expression level, quantitative measurement of CAR+γδ T cells, serum level of one or more cytokines and/or serum proteins, and minimal residual disease (MRD). In embodiments, the one or more cytokines/serum proteins are selected from the group comprising or consisting of INFY, GM-CSF, IL-2, IL-7, IL-15, TNFα, IL-1β, IL-6, IL-8, IL-10, MIP1α, MIP1β, CRP, ferritin, monocyte chemotactic protein-1 (MCP-1), CXCL9, CXCL10, CXCL11, CCL5, IL-5, IL-IRA, IL-18, soluble MICA, IL-10, IL-4, IL-13, IL-17, CCL2, CXCL12, CCL17, and CCL22. In exemplary embodiments, the one or more cytokines are IL-2 and/or IL-8. In embodiments, the subject methods may further comprise measuring CAR transgene expression level via quantitative polymerase chain reaction (qPCR). In embodiments, the quantitative measurement of CAR+γδ T cells is determined via flow cytometry. In embodiments, the subject methods further comprise conducting analysis of MRD via immunosequencing methodology.

In a further aspect, the subject methods may further comprise administering a secondary treatment regimen based at least in part on monitoring one or more of the foregoing biomarkers. In embodiments, the secondary treatment regimen comprises one or more additional doses of anti-CD20 CAR γδ T cells. In embodiments, the one or more additional doses of anti-CD20 CAR γδ T cells are administered without an additional LD regimen. In embodiments, the one or more additional doses of anti-CD20 CAR γδ T cells are administered following an additional LD regimen. In embodiments, the one or more additional doses of anti-CD20 CAR γδ T cells are administered as a consolidation therapy to kill any cancer cells that may be left in the body, preferably wherein the one or more additional doses of anti-CD20 CAR γδ T cells are administered following an additional LD regimen. In embodiments, the one or more additional doses comprise an increased amount of anti-CD20 CAR γδ T cells. In embodiments, the one or more additional doses comprise a decreased amount of anti-CD20 CARγδ T cells. In embodiments, the one or more additional doses comprise the same amount of anti-CD20 CAR γδ T cells. In embodiments, the one or more additional doses comprise anti-CD20 CAR γδ T cells derived from the same donor. In embodiments, the one or more additional doses comprise anti-CD20 CAR γδ T cells derived from a different donor. In embodiments, the follow-on treatment regimen further comprises administration of cyclophosphamide, doxorubicin hydrochloride (hydroxydaunorubicin), vincristine sulfate, and prednisone (CHOP).

In embodiments, the anti-CD20 CAR can comprise the amino acid sequence of SEQ ID NO: 41. In embodiments, the anti-CD20 γδ T cells can further comprise a nucleic acid sequence encoding the amino acid sequence of SEQ ID NO: 41. In embodiments, the anti-CD20 γδ T cells can further comprise a nucleic acid sequence encoding the CAR and having the sequence of SEQ ID NO: 46.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIGS. 1A-1D illustrate Fluorodeoxyglucose (FDG)-positron emission tomography (PET) images obtained on a subject before (FIG. 1A and FIG. 1C) and after (FIG. 1B and FIG. 1D) treatment with 3×10⁷ anti-CD20 CAR γδ T cells.

FIGS. 2A-2D illustrate FDG-PET images obtained on another subject before (FIG. 2A and FIG. 2C) and after (FIG. 2B and FIG. 2D) treatment with 3×10⁷ anti-CD20 CAR γδ T cells.

FIGS. 3A-3D illustrate FDG-PET images obtained on another subject before (FIG. 3A and FIG. 3C) and after (FIG. 3B and FIG. 3D) treatment with 1×10⁸ anti-CD20 allogeneic γδ CAR T cells.

FIGS. 4A-4C are graphs showing serum levels of various cytokines in subjects prior to lymphodepletion, after lymphodepletion but prior to infusion of anti-CD20 allogeneic γδ CAR T cells, and after infusion of anti-CD20 allogeneic γδ CAR T cells.

FIGS. 5A-5D illustrate flow cytometry analysis indicative of in vivo expansion of anti-CD20 allogeneic γδ CAR T cells.

FIG. 6 provides a summary of the safety findings to date in a first in human study of anti-CD20 allogeneic γδ CAR T cells in adults with various B-cell malignancies.

FIG. 7 provides a swimmer plot for patients treated to date along with a summary of the independent radiographic assessments of these patients. TH, triple hit; DH, double hit; DLBCL, diffuse large B-cell lymphoma; HGBCL, high grade B-cell lymphoma; MCL, mantle cell lymphoma; mCR: metabolic complete response (PET negative).

### DETAILED DESCRIPTION

### I. Definitions

For purposes of interpreting this specification, the following definitions will apply, and whenever appropriate, terms used in the singular will also include the plural and vice versa. In the event that any definition set forth conflicts with any document incorporated herein by reference, the definition set forth below shall control. Unless defined otherwise, all technical and scientific terms used herein have the same meaning as commonly understood by one of ordinary skill in the art to which the disclosure pertains.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “w/v” refers to the weight of the component in a given volume of solution.

“Ranges”: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The terms “patient,” “subject,” “individual,” and the like are used interchangeably herein, and refer to any animal amenable to the methods described herein. In certain non-limiting embodiments, the patient, subject or individual is a human.

As used herein, the term “agent” refers to any protein, nucleic acid molecule (including chemically modified nucleic acids), compound, antibody, small molecule, organic compound, inorganic compound, other molecule of interest, or cell (e.g., cell engineered to express a chimeric antigen receptor). Agent can include a therapeutic agent, a diagnostic agent or a pharmaceutical agent. A therapeutic or pharmaceutical agent is one that alone or together with an additional agent induces the desired response (such as inducing a therapeutic or prophylactic effect when administered to a subject, including treating a subject suffering from cancer, or other disease/condition.

The term “diagnosis”, or “diagnosing” as used herein refers to the process of identifying a disease, such as cancer, by its signs, symptoms, and/or results of various tests. A conclusion reached through such a process is a diagnosis. Forms of testing commonly performed include blood tests, medical imaging, urinalysis, biopsy, and the like.

The term “therapeutically effective amount”, or simply “effective amount” refers to the amount of an agent or composition (e.g., composition comprising an agent) that will elicit a biological or medical response of a tissue, system, or subject that is being sought by the researcher, veterinarian, medical doctor or other clinician. The term “therapeutically effective amount” includes that amount of an agent, or a composition comprising an agent, that, when administered, is sufficient to prevent development of, or alleviate to some extent, one or more of the signs or symptoms of the disorder or disease (e.g., hematological or solid tumor) being treated. The therapeutically effective amount will vary depending on the composition, the disease and its severity and the age, weight, etc., of the subject to be treated.

To “treat” a disease as the term is used herein, means to decrease or reduce the frequency or severity of at least one sign or symptom of a disease or disorder experienced by a subject. In one example, a therapy (e.g., administration of a therapeutic agent of the present disclosure) treats a disease or condition by decreasing one or more signs or symptoms associated with the disease or condition, for example as compared to the response in the absence of the therapy. For example, administration of a therapeutic agent may provide an anti-tumor effect that decreases one or more signs or symptoms associated with cancer.

As used herein, the term “administration” means to provide or give a subject one or more agents, such as an agent that treats one or more signs or symptoms associated with a condition/disorder or disease including but not limited to cancer (e.g., lymphoma), viral infection, bacterial infection, etc., by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes. Administration “in combination with” one or more further therapeutic agents includes simultaneous (concurrent) and sequential administration in any order.

The term “pharmaceutically acceptable”, as used herein, refers to a material, including but not limited, to a salt, carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained. The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more agents, such as one or more modulatory agents. In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical agents to be administered can contain minor amounts of non-toxic auxiliary substances, such as wetting or emulsifying agents, preservatives, and pH buffering agents and the like, for example sodium acetate or sorbitan monolaurate, sodium lactate, potassium chloride, calcium chloride, and triethanolamine oleate. For example, the present invention provides a pharmaceutical composition comprising a pharmaceutically acceptable excipient and an, e.g., γδ, T cell, preferably a γδ T cell engineered to express a CAR directed to CD20, as described herein.

As used herein, the term “pharmacodynamic (PD) biomarker” and/or “pharmacokinetic (PK) biomarker” refers to one or more measurable indicators associated with administration of a therapeutic agent to a subject. Broadly speaking, a PK marker relates to how the body affects a therapeutic agent, whereas a PD marker relates to how the therapeutic agent affects a subject.

As used herein, the term “relapsed/refractory B cell malignancy” encompasses any B cell lymphoma that is ultimately non-responsive to treatment including but not limited to Non-Hodgkin lymphoma (NHL); chronic lymphocytic leukemia/small lymphocytic lymphoma (CLL/SLL), acute lymphocytic leukemia (ALL), and acute myeloid leukemia (AML). Accordingly in embodiments the B cell malignancy may be selected from the group comprising or consisting of NHL, CLL, ALL and/or AML. In embodiments the B cell malignancy may be a form of NHL selected from the group comprising or consisting of diffuse large B cell lymphoma (DLBCL), mantle cell lymphoma (MCL), transformed follicular lymphoma (tFL), primary mediastinal (thymic) large B cell lymphoma (PMBCL), high-grade B cell lymphomas, Burkitt lymphoma, follicular lymphoma (FL), and marginal zone lymphoma (MZL).

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antigen” or “Ag” as used herein is defined as a molecule that provokes an immune response. This immune response may involve either antibody production, or the activation of specific immunologically-competent cells, or both. The skilled artisan will understand that any macromolecule, including proteins or peptides, can serve as an antigen.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies (including agonist, antagonist, neutralizing antibodies, full length or intact monoclonal antibodies), antibody compositions with polyepitopic specificity, multivalent antibodies, multispecific antibodies (e.g., bispecific antibodies so long as they exhibit the desired biological activity), formed from at least two intact antibodies, diabodies, single domain antibodies (sdAbs), as long as they exhibit the desired biological or immunological activity, Fv, Fab and F(ab), as well as single chain antibodies and humanized antibodies (Harlow et ah, 1999, In: Using Antibodies: A Laboratory Manual, Cold Spring Harbor Laboratory Press, NY: Harlow et ah, 1989, In; Antibodies: A Laboratory Manual, Cold Spring Harbor, N.Y.; Houston et ah, 1988, Proc. Nat Acad. Sci. USA 85:5879-5883: Bird et ah, 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in antibody molecules in their naturally occurring conformations.

An “antibody light chain,” as used herein, refers to the smaller of the two types of polypeptide chains present in antibody molecules in their naturally occurring conformations. K and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “epitope” includes any protein determinant, lipid or carbohydrate determinant capable of specific binding to an immunoglobulin or receptor, for example a T-cell receptor. Epitopic determinants usually consist of active surface groupings of molecules such as amino acids, lipids or sugar side chains and usually have specific three-dimensional structural characteristics, as well as specific charge characteristics.

The term “specifically binds”, as used herein refers to a receptor (which can include but is not limited to an antibody or antibody fragment) which recognizes a specific molecule/ligand, but does not substantially recognize or bind other molecules in a sample. For example, a receptor that specifically binds to a molecule from one species may also bind to that molecule from one or more other species. But, such cross-species reactivity does not itself alter the classification as specific. In another example, a receptor that specifically binds to a molecule may also bind to different allelic forms of the molecule. However, such cross reactivity does not itself alter the classification as specific. In some instances, the terms “specific binding” or “specifically binding,” can be used in reference to the interaction of a protein (or a peptide) with a second chemical species, to mean that the interaction is dependent upon the presence of a particular structure (e.g., an antigenic determinant or epitope) on the chemical species; for example, a receptor recognizes and binds to a specific a structure rather than to proteins generally. If receptor is specific for epitope “A”, the presence of a molecule containing epitope A (or free, unlabeled A), in a reaction containing labeled “A” and the receptor, will reduce the amount of labeled A bound to the receptor.

In some embodiments, specific binding can be characterized by an equilibrium dissociation constant of at least about 1×10⁻⁸ M or less (e.g., a smaller KD denotes a tighter binding). Methods for determining whether two molecules specifically bind are well known in the art and include, for example, equilibrium dialysis, surface plasmon resonance, and the like.

The term “anti-tumor effect” as used herein, refers to a biological effect which can be manifested by a decrease in tumor volume, a decrease in the number of tumor cells, a decrease in metabolic activity of the tumor cells (e.g., PET signal), a decrease in the number of metastases, an increase in life expectancy, or amelioration of various physiological symptoms associated with the cancerous condition.

As used herein, the term “autologous” is meant to refer to any material derived from an individual which is later to be re-introduced into the same individual.

As used herein, the term “allogeneic” refers to material derived from an animal which is later introduced into a different animal of the same species.

The term “γδ T-cells” or “gamma delta T-cells” as used herein refers to a subset of T-cells that express a distinct T-cell receptor (TCR), namely γδ TCR, on their surface, composed of one γ-chain and one δ-chain. The term “γδ T-cells” specifically includes all subsets of γδ T-cells, including, without limitation, Vδ1, Vδ2, and Vδ3 γδ T cells, as well as naive, effector memory, central memory, and terminally differentiated γδ T-cells. As a further example, the term “γδ T-cells” includes Vδ4, Vδ5, Vδ7, and Vδ8 γδ T cells, as well as Vγ2, Vγ3, Vγ5, Vγ8, Vγ9, Vγ10, and Vγ11 65 δ T cells.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., IRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Expression cassette” refers to a nucleic acid comprising expression control sequences operatively linked to a nucleic acid encoding a transcript or polypeptide to be expressed. An expression cassette comprises sufficient cis-acting elements for expression; other elements for expression can be supplied by the host cell or in an in vitro expression system. Expression cassettes can be a component of a vector such as a cosmid, a plasmid (e.g., naked or contained in a liposome), or a virus (e.g., lentivirus, retrovirus, adenovirus, and adeno-associated virus). An expression cassette can be in a host cell, such as a γδ T cell.

### Chimeric Antigen Receptor Constructs

Aspects of the invention include nucleic acids encoding CARs, and constructs and vectors containing such nucleic acids. In some cases, the nucleic acid is a, e.g., heterologous, component of an expression cassette. In some embodiments, the nucleic acid is a, e.g., heterologous, component of a retroviral vector. In some embodiments, the nucleic acid is a, e.g., heterologous, component of a γδ T cell. In some embodiments, the nucleic acid is a, e.g., heterologous, component of a γ⁺ T cell and/or a δ⁺ T cell.

Described herein are nucleic acids encoding a CAR binding domain that specifically binds to a tumor associated antigen (TAA) expressed on a surface of a hematological tumor cell. In preffered embodiments, the binding domain is a CD20 binding domain, such as a CD20 binding domain described in U.S. Patent Appl. No. 2009/0035322 and WO 2020/072536A9, the contents of each of which are incorporated by reference in the entirety and for all purposes and in particular for the binding domains, antibodies, antibody fragments, complementarity determining regions, polypeptides containing said complementarity determining regions, nucleic acids encoding for said complementarity determining regions, and epitope specificities and assays for determining epitope specificity described therein. Typically, the region encoding the binding domain is 5′ of a linker region (e.g., a region encoding a CD8α hinge domain).

Exemplary CD20 binding domains include but are not limited to binding domains that selectively bind to an epitope within CD20 bound by, or that competes for binding with, 3B9, 3H7, 2B7, 9C11, or 10F2; or 3B9, 3H7, 2B7, or 9C11; or 3H7. Additionally or alternatively, the CD20 binding domain can comprise the complementary determining regions of an anti-CD20 antibody selected from the group consisting of 3B9, 3H7, 2B7, 9C11, and 10F2; selected from the group consisting of 3B9, 3H7, 2B7, and 9C11; or comprise the complementary determining regions of an anti-CD20 antibody selected from the group consisting of 3H7. The present disclosure also contemplates CD20 binding domains that compete for binding with a sequence provided herein.

One can determine whether a CD20 binding domain binds to the same epitope as, or competes for binding with, a reference antibody or binding domain by using known methods. For example, to determine if a test antibody binds to the same epitope as a reference binding domain, the reference binding domain can be allowed to bind to CD20 under saturating conditions. Next, the ability of a test binding domain to bind to CD20 molecule can be assessed. If the test binding domain is able to bind to CD20 following saturation binding with the reference binding domain, it can be concluded that the test binding domain binds to a different epitope than the reference binding domain. On the other hand, if the test binding domain is not able to bind to CD20 following saturation binding with the reference binding domain, then the test binding domain may bind to the same epitope as the epitope bound by the reference binding domain.

To determine if a binding domain competes for binding with a reference binding domain, the above-described binding methodology is performed in two orientations: In a first orientation, the reference binding domain is allowed to bind to CD20 under saturating conditions followed by assessment of binding of the test binding domain to the CD20 molecule. In a second orientation, the test binding domain is allowed to bind to a CD20 molecule under saturating conditions followed by assessment of binding of the reference binding domain to the CD20 molecule. If, in both orientations, only the first (saturating) binding domain is capable of binding to the CD20 molecule, then it is concluded that the test binding domain and the reference binding domain compete for binding to CD20. As will be appreciated by a person of ordinary skill in the art, a binding domain that competes for binding with a reference binding domain may not necessarily bind to the identical epitope as the reference binding domain, but may sterically block binding of the reference binding domain by binding an overlapping or adjacent epitope.

Two binding domains bind to the same or an overlapping epitope if each competitively inhibits (blocks) binding of the other to the antigen. That is, a 1-, 5-, 10-, 20-or 100-fold excess of one binding domain inhibits binding of the other by at least 50%, for example, 75%, 90% or even 99% as measured in a competitive binding assay (see, e.g., Junghans et al., Cancer Res. 1990 50:1495-1502). Alternatively, two binding domains have the same epitope if essentially all amino acid mutations in the antigen that reduce or eliminate binding of one binding domain also reduce or eliminate binding of the other. Two binding domains have overlapping epitopes if some amino acid mutations that reduce or eliminate binding of one binding domain also reduce or eliminate binding of the other.

Additional routine experimentation (e.g., peptide mutation and binding analyses) can then be carried out to confirm whether the observed lack of binding of the test binding domain is in fact due to binding to the same epitope as the reference binding domain or if steric blocking (or another phenomenon) is responsible for the lack of observed binding. Experiments of this sort can be performed using ELISA, RIA, surface plasmon resonance, flow cytometry or any other quantitative or qualitative binding assay available in the art.

In embodiments, a CD20 binding domain as herein disclosed binds a CD20 epitope that is different from a CD20 epitope bound by rituximab. In embodiments, a CD20 binding domain as herein disclosed binds a CD20 epitope that is different from a CD20 epitope bound by ocrelizumab. In some embodiments, a CD20 binding domain as herein disclosed binds a same or overlapping CD20 epitope bound by ocrelizumab. In embodiments, a CD20 binding domain as herein disclosed binds a CD20 epitope that is different from a CD20 epitope bound by ofatumumab, or obinutuzumab, or veltuzumab. In embodiments, a CD20 binding domain as herein disclosed bind a same or overlapping CD20 epitope bound by ofatumumab, or obinutuzumab, or veltuzumab. In embodiments, a CD20 binding domain as herein disclosed binds a CD20 epitope that is different from one or more anti-CD20 mAbs described by Luo et al. (2021) Scientific Reports, 11(3255). In embodiments, a CD20 binding domain as herein disclosed binds a same or overlapping CD20 epitope bound by one or more anti-CD20 mAbs described by Luo et al. (2021) Scientific Reports, 11 (3255). In embodiments, a CD20 binding domain as herein disclosed binds a CD20 epitope that is different from one or more anti-CD20 mAbs described by Casan et al. (2018) Hum Vaccin Immunother, 14 (12): 2820-2841. In embodiments, a CD20 binding domain as herein disclosed binds a same or overlapping CD20 epitope bound by one or more anti-CD20 mAbs described by Casan et al. (2018) Hum Vaccin Immunother, 14(12): 2820-2841.

The present disclosure provides antibodies and CARs with “substantial identity” or “substantial similarity” to the sequences provided herein in the CDR or framework regions. The term “substantial identity” or “substantially identical,” when referring to a nucleic acid or fragment thereof, indicates that, when optimally aligned with another nucleic acid (or the complementary strand of the other nucleic acid), there is nucleotide sequence identity in %, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or 100% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or 100% sequence identity. In some aspects, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24:307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443 45, herein incorporated by reference. A “moderately conservative” replacement is any change having a nonnegative value in the PAM250 log-likelihood matrix.

Sequence identity and/or similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Sequences also can be compared using the Smith-Waterman homology search algorithm using an affine gap search with a gap open penalty of 12 and a gap extension penalty of 2, BLOSUM matrix of 62. Another preferred algorithm when comparing a sequence disclosed herein to a database containing a large number of sequences from different organisms is the computer program BLAST, especially BLASTP or TBLASTN, using default parameters. See, e.g., Altschul et al. (1990) J. Mol. Biol. 215:403-410 and (1997) Nucleic Acids Res. 25:3389-3402, each of which is herein incorporated by reference.

Provided herein are anti-CD20 CARs comprising variants of any of the HCVR, LCVR, and/or CDR amino acid sequences disclosed herein having one or more substitutions (e.g., conservative substitutions). For example, the present disclosure includes anti-CD20 CARs having HCVR, LCVR, and/or CDR amino acid sequences with, e.g., 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 amino acid substitutions relative to any of the HCVR, LCVR, and/or CDR (e.g., HCDR1, HCDR2, HCDR3, LCDRI, LCDR2, or LCDR3) amino acid sequences disclosed herein. For example, an anti-CD20 CAR can comprise 20, 19, 18, 17, 16, 15, 14 13, 12, 11, 10, 9, 8, 7, 6, 5, 4, 3, 2, or 1 amino acid substitutions (e.g., conservative amino acid substitutions) relative to any of the HCVR, LCVR, and/or CDR (e.g., HCDR1, HCDR2, HCDR3, LCDR1, LCDR2, or LCDR3) amino acid sequences disclosed herein.

In some embodiments, the isolated nucleic acid encodes an anti-CD20 binding domain having a heavy chain complementary determining region 3 (HCDR3) and a light chain CDR3 (LCDR3), wherein the HCDR3 and LCDR3 are selected from the group consisting of SEQ ID NO: 1 (AKDPSYGSGSYHSYYGMDV) and 2 (QQRFNWPLT); 3 (VKDFHYGSGSNYGMDV) and 4 (QQSNDWPLT); and 5 (TKDGSYGHFYSGLDV) and 6 (QQRYYWPLT).

In some embodiments, the isolated nucleic acid encodes an anti-CD20 binding domain having a heavy chain variable region (HCVR) sequence and a light chain variable region (LCVR) sequence, wherein the HCVR and LCVR sequences are selected from the group consisting of SEQ ID NO: 7 (EEQLVESGGDLVQPGRSLRLSCAASGFTFHDYTMH WVRQAPGKGLEWVSGISWNSGSLGYADSVKGRFTISRDNAKKSLYLQMNSLRAEDTAL YYCAKDPSYGSGSYHSYYGMDVWGQGTTVTVSS) and 8 (EIVLTQSPATLSLSPGE RATLSCWASQSISRYLVWYQQKCGQAPRLLIYEASKRATGIPVRFSGSGSGTDFTLTISSL ESEDFAVYYCQQRFNWPLTFGGGTKVEIK); 9 (EVQLAESGGDLVQSGRSLRLSCAAS GITFHDYAMHWVRQPPGKGLEWVSGISWNSDYIGYADSVKGRFTISRDNAKKSLYLQM NSLRPDDTALYYCVKDFHYGSGSNYGMDVWGQGTTVTVSP) and 10 (EIVMTQSPATL SMSPGERATLSCRASQSVSRNLAWYQQKVGQAPRLLISGASTRATGIPARFSGSGSGTEF TLTINSLQSEDFAVYYCQQSNDWPLTFGQGTRLEIK); and 11 (EVQLVESGGGLVQPGR SLRLSCAASGFTFYDYAMHWVRQAPGKGLEWVSGISWNSDTIGYADSVKGRFTISRDN AKNSLYLQMNSLRAEDTALYYCTKDGSYGHFYSGLDVWGQGTTVTVSS) and 12 (EIVLTQSPATLSLSPGERATLSCRASQSVSSYLAWYQQKPGQAPRLLIYVASNRATGIPA RFSGSGSGTDFTLTISSLEPDDFAVYYCQQRYYWPLTFGGGTKVEIK).

In some embodiments, the isolated nucleic acid encodes an anti-CD20 binding domain having a heavy chain complementary determining region 3 (HCDR3) domain and a light chain CDR3 (LCDR3) domain, wherein the HCDR3 domain comprises an amino acid sequence of the formula X1-X2-X3-X4-X5-X6-X7-X8-X9-X10-X11-X12-X13-X14-X15-X16-X17-X18-X19, wherein X1=A, V or T; X2=K; X3=D; X4=P, F or G; X5=S or H; X6=Y; X7-G; X8-S or H; X9-G or F; X10=S or Y; X11=Y, N or S; X12=Y, G or H; X13=G, L or S; X14=Y, M or D; X15=Y, D or V; X16 =G, V or absent; X17=M or absent; X18=D or absent; X19=V or absent; and the LCDR3 domain comprises an amino acid sequence of the formula X1-X2-X3-X4-X5-X6-X7-X8-X9, wherein X1=Q; X2=Q; X3=R or S; X4=N, Y or F; X5=N, D, or Y; X6=W; X7=P; X8-L; X9=T.

In some embodiments, the isolated nucleic acid encodes an anti-CD20 binding domain having a heavy chain variable region (HCVR) sequence and a light chain variable region (LCVR) sequence, wherein the HCVR and LCVR sequences are SEQ ID NO: 13 (EVQLVESGGGLVQPGRSLRLSCAASGFTFYDYAMHWVRQAPGKGLEWVSGISWNSGY IGYADSVKGRFTISRDNAKNSLYLQMNSLRAEDTALYYCAKDNSYGKFYYGLDVWGQ GTTVTVSS) and 14 (EIVMTQSPATLSVSPGERTTLSCRASQSVSSNLAWYLQKPGQAPR LLIYGASTRATGIPARFSGSGSGTEFILTISSLQSEDFAVYYCQQYNNWPITFGQGTRLEI).

In some embodiments, the isolated nucleic acid encodes an anti-CD20 binding domain that binds the same epitope as, competes with, or is an anti-CD20 binding domain having heavy chain complementarity determining regions (HCDR) and a light chain complementarity determining regions (LCDR), wherein the HCDR and LCDR sequences are the HCVR sequences of SEQ ID NO: 13 and the LCVR sequences of SEQ ID NO: 14 respectively.

In some embodiments, the isolated nucleic acid encodes an anti-CD20 binding domain that binds the same epitope as, competes with, or is an anti-CD20 binding domain having an HCDRI that is or comprises SEQ ID NO: 15 (GFTFYDYA), an HCDR2 that is or comprises SEQ ID NO: 16 (ISWNSGYI), and/or an HCDR3 that is or comprises SEQ ID NO: 17 (AKDNSYGKFYYGLDV). In some embodiments, the isolated nucleic acid encodes an anti-CD20 binding domain that binds the same epitope as, competes with, or is an anti-CD20 binding domain having an LCDRI that is or comprises SEQ ID NO: 18 (QSVSSN), an LCDR2 that is or comprises SEQ ID NO: 19 (GAS), and/or an LCDR3 that is or comprises SEQ ID NO: 20 (QQYNNWPIT). In some embodiments, the isolated nucleic acid encodes an anti-CD20 binding domain that binds the same epitope as, competes with, or is an anti-CD20 binding domain having an HCDR1 that is or comprises SEQ ID NO:15, an HCDR2 that is or comprises SEQ ID NO:16, an HCDR3 that is or comprises SEQ ID NO: 17, an LCDRI that is or comprises SEQ ID NO: 18, an LCDR2 that is or comprises SEQ ID NO: 19, and an LCDR3 that is or comprises SEQ ID NO: 20. In some embodiments, the isolated nucleic acid encodes an anti-CD20 binding domain having an HCDR1 comprising SEQ ID NO:15, an HCDR2 comprising SEQ ID NO:16, an HCDR3 comprising SEQ ID NO: 17, an LCDRI comprising SEQ ID NO: 18, an LCDR2 comprising SEQ ID NO: 19, and an LCDR3 comprising SEQ ID NO: 20.

Exemplary binding domains described herein typically comprise, in order from the amino to carboxy terminus, a heavy chain region followed by a light chain region (VH-VL). Where a certain order of of VH and VL region in the binding domain is explicitly or implicitly described, the present disclosure is also understand to describe the alternate embodiment in which the order of VH and VL regions are reversed, e.g., in an scFV or a CAR comprising an scFv binding domain. Thus, description of a VH-VL order also describes the alternate VL-VH order, e.g., in an scFV or a CAR comprising an scFv binding domain. Moreover, description of a VL-VH order also describes the alternate VH-VL order, e.g., in an scFV or a CAR comprising an scFv binding domain.

Generally, the CAR encoding nucleic acids described herein include an extracellular linker portion that encodes a peptide linker that links the binding domain to a transmembrane domain. Exemplary linker portions include, without limitation, a linker portion that encodes the CD8α hinge domain, e.g., SEQ ID NO: 21 (PTPAPTIASQPLSLRPEACRPAAGGAVHTRGLDFACDIY) or SEQ ID NO:22 (TTTPAPRP PTPAPTIASQPLSLRPEACRPAAGGAVHTRGLDFACDIY). Typically, the region encoding the peptide linker (e.g., CD8α hinge domain) is 3′ of the region encoding the binding domain and 5′ of a region encoding a transmembrane domain.

The CAR encoding nucleic acids described herein include a transmembrane domain. The transmembrane domain can link an extracellular antigen binding domain, e.g., and hinge, to one or more intracellular signaling components. For example, the transmembrane domain can link an antigen binding domain, e.g., and hinge, to a CD3ζ signaling domain and optionally with one or two costimulation endodomains. Exemplary transmembrane domains include without limitation a CD8a transmembrane domain, e.g., SEQ ID NO: 23 (IWAPLAGTCGVLLLSLVITLYC). Typically, the region encoding the transmembrane domain (e.g., CD8α transmembrane domain) is 3′ of the region encoding the peptide linker (e.g., CD8α hinge domain) and 5′ of a region encoding one or more cytoplasmic domains.

In some embodiments, the isolated nucleic acid encodes a cytoplasmic region containing one or more cytoplasmic domains. The region encoding the cytoplasmic region is typically 3′ of the region encoding the transmembrane domain. The cytoplasmic domains are typically signaling domains that provide an activating signal for γδ T cell proliferation, cytotoxic activity, and/or pro-inflammatory cytokine expression (e.g., TNF-α or IFNγ). An exemplary cytoplasmic domain is a CD3ζ signaling domain. In some embodiments, the CD3ζ signaling domain is or comprises SEQ ID NO: 24 (RVKFSRSADAPAYQQGQNQLYNELNLGR REEYDVLDKRRGRDPEMGGKPQRRKNPQEGLYNELQKDKMAEAYSEIGMKGERRRGK GHDGLYQGLSTATKDTYDALHMQALPPR). In some embodiments, the CD33 signaling domain is or comprises SEQ ID NO: 25 (RVKFSRSADAPAYQQGQNQLYNELNLGRREEYDVLDKRRGRDPEMGGKPRRKNPQE GLYNELQKDKMAEAYSEIGMKGERRRGKGHDGLYQGLSTATKDTYDALHMQALPPR). In some embodiments, the cytoplasmic region contains multiple (e.g., 2, 3, 4, 5, or 6) signaling domains, such as multiple (e.g., 2, 3, 4, 5, or 6) CD3ζ signaling domains, e.g., each independently selected from SEQ ID NO: 24 and 25. In some embodiments, the cytoplasmic region contains multiple (e.g., 2, 3, 4, 5, or 6) non-CD3ζ signaling domains and a CD3ζ signaling domain. In some embodiments, the cytoplasmic region contains a non-CD3ζ signaling domain and multiple (e.g., 2, 3, 4, 5, or 6) CD3ζ signaling domains.

The cytoplasmic region can contain one or more costimulatory domains. A region encoding one or more costimulatory domains can be 5′ or 3′ of a region encoding a signaling domain. In some embodiments, the region encoding one or more costimulatory endodomains is 5′ of the region encoding a signaling domain. In some embodiments, a region encoding one or more costimulatory endodomains is 5′ of a signaling domain and an additional region encoding one or more costimulatory endodomains is 3′ of the signaling domain. Exemplary costimulation endodomains include, without limitation, CD28; CD137 (4-1BB); CD278 (ICOS); CD27; CD134 (OX40); Dap10; Dap12; DNAm-1; 2B4; a SLAM domain; and TLR2 costimulation endodomains, and combinations thereof.

In some embodiments, the construct encodes at least one 4-1BB costimulation endodomain, and optionally a second costimulation endodomain selected from a 4-1BB, 2B4, ICOS, CD28, and CD27 costimulation endodomain. In some embodiments, the construct encodes at least two 4-1BB costimulation endodomains, or two 4-1BB costimulation endodomains in combination with one, two, three, or four, or more, costimulation endodomains selected from a 4-1BB, ICOS, CD28, and CD27. In some embodiments, the 4-1BB costimulation endodomain comprises SEQ ID NO: 26(KRGRKKLLYIFKQPFMRPVQTT QEEDGCSCRFPEEEEGGCEL).

In some embodiments, the construct encodes one CD27 costimulation endodomain, and optionally a second costimulation endodomain selected from a 4-1BB, ICOS, CD28, and CD27 costimulation endodomain. In some embodiments, the construct encodes a CD27 costimulation endodomain, and a 4-1BB costimulation endodomain. In some embodiments, the construct encodes two CD27 costimulation endodomains. In some embodiments, the CD27 costimulation endodomain comprises SEQ ID NO: 27 (QRRKYRSNKGESPVEPAEPCHYSCPREEEGSTIPIQED YRKPEPACSP).

In some embodiments, the construct encodes a secretion signal, e.g., SEQ ID NO: 28 (MALPVTALLLPLALLLHAARP) operably linked to facilitate secretion of a C-terminal polypeptide, such as a cytokine that supports the activation, cytotoxicity, and/or persistence of a T cell (e.g., CAR-T cell). In some embodiments, the construct encodes a secretion signal, e.g., SEQ ID NO: 28 operably linked to facilitate secretion of a common gamma chain cytokine such as IL-15 or an active fragment thereof, e.g., SEQ ID NO: 29 (NWVNVISDLKKIED LIQSMHIDATLYTESDVHPSCKVTAMKCFLLELQVISLESGDASIHDTVENLIILANNSLSS NGNVTESGCKECEELEEKNIKEFLQSFVHIVQMFINTS). Exemplary common gamma chain cytokines include IL-2 and IL-15. In some embodiments, the common gamma chain cytokine is selected from IL-2, IL-7, and IL-15. In some embodiments, the common gamma chain cytokine is IL-15. IL-15 sequences, including codon optimized nucleic acid sequences encoding sIL 15, are disclosed herein and in WO 2007/037780.

In some embodiments, the construct encodes one or more multi-cistronic linker regions, e.g., between a signaling domain and/or costimulation endodomain and a secretion signal operably linked to facilitate secretion of a cytokine. A multi-cistronic linker region is a region of polypeptide sequence or RNA sequence that facilitates the production of multiple discrete polypeptides from a single transcription product. In some embodiments, the multi-cistronic linker region encodes a cleavage sequence. Suitable cleavage sequences include self-cleavage sequences such as a P2A, F2A, E2A, or T2A cleavage sequence and/or sequences that are cleaved by an endogenous protease, such as furin.

In some embodiments, the cleavage sequence is a P2A cleavage sequence. In some embodiments, the cleavage sequence is a furin cleavage sequence. In some embodiments, the cleavage sequences are a P2A and a furin cleavage sequence. In some embodiments, the cleavage sequence is the P2A cleavage sequence of SEQ ID NO: 30 (SGSGATNFSLLKQAGDVEENPGP). In some embodiments, the cleavage sequence is a furin cleavage sequence of SEQ ID NO: 31 (RAKR). In some embodiments, the cleavage sequence is a P2A+furin cleavage sequence of SEQ ID NO: 32 (RAKRSGSGATNFSLLKQAG DVEENPGP).

In some embodiments, the cleavage sequence is or comprises a P2A cleavage sequence of SEQ ID NO: 33 (ATNFSLLKQAGDVEENPGP). In some embodiments, the cleavage sequence is or comprises an F2A cleavage sequence of SEQ ID NO: 34 (VKQTLNNFDLLKLAGDVESNPGP). In some embodiments, the cleavage sequence is or comprises an E2A cleavage sequence of SEQ ID NO: 35 (QCTNYALLKLAGDVESNPGP). In some embodiments, the cleavage sequence is or comprises an T2A cleavage sequence of SEQ ID NO: 36 (EGRSLLTCGDVEENPGP). In certain aspects, multiple self-cleavage sequences can be encoded carboxy terminal to a signaling and/or costimulatory domain and amino-terminal to an encoded secreted cytokine (e.g., common gamma chain cytokine such as IL-15), preferably wherein the multiple self cleavage sequences are independently selected from the group consisting of a P2A cleavage sequence, a T2A cleavage sequence, an E2A cleavage sequence, and an F2A cleavage sequence. In certain aspects, one or more self-cleavage sequences and one or more sequences cleaved by an endogenous protease are encoded in a construct described herein. In certain embodiments, a endogenous protease recognition site is encoded amino terminal to a self cleavage sequence.

In some embodiments, the multi-cistronic linker region encodes an internal ribosome entry site. An exemplary internal ribosome entry site is encoded by SEQ ID NO: 37 (CTAACGTTACTGGCCGAAGCCGCTTGGAATAAGGCCGGTGTGCGTTTGTCTATATGT TATTTTCCACCATATTGCCGTCTTTTGGCAATGTGAGGGCCCGGAAACCTGGCCCTGT CTTCTTGACGAGCATTCCTAGGGGTCTTTCCCCTCTCGCCAAAGGAATGCAAGGTCT GTTGAATGTCGTGAAGGAAGCAGTTCCTCTGGAAGCTTCTTGAAGACAAACAACGTC TGTAGCGACCCTTTGCAGGCAGCGGAACCCCCCACCTGGCGACAGGTGCCTCTGCG GCCAAAAGCCACGTGTATAAGATACACCTGCAAAGGCGGCACAACCCCAGTGCCAC GTTGTGAGTTGGATAGTTGTGGAAAGAGTCAAATGGCTCTCCTCAAGCGTATTCAAC AAGGGGCTGAAGGATGCCCAGAAGGTACCCCATTGTATGGGATCTGATCTGGGGCC TCGGTGCACATGCTTTACATGTGTTTAGTCGAGGTTAAAAAAACGTCTAGGCCCCCC GAACCACGGGGACGTGGTTTTCCTTTGAAAAACACGATGATA).

Another exemplary internal ribosome entry site is encoded by SEQ ID NO: 38 (AGCAGGTTTCCCCAACTGACACAAAACGTGCAACTTGAAACTCCGCCTGGTCTTTC CAGGTCTAGAGGGGTAACACTTTGTACTGCGTTTGGCTCCACGCTCGATCCACTGGC GAGTGTTAGTAACAGCACTGTTGCTTCGTAGCGGAGCATGACGGCCGTGGGAACTCC TCCTTGGTAACAAGGACCCACGGGGCCAAAAGCCACGCCCACACGGGCCCGTCATG TGTGCAACCCCAGCACGGCGACTTTACTGCGAAACCCACTTTAAAGTGACATTGAAA CTGGTACCCACACACTGGTGACAGGCTAAGGATGCCCTTCAGGTACCCCGAGGTAA CACGCGACACTCGGGATCTGAGAAGGGGACTGGGGCTTCTATAAAAGCGCTCGGTT TAAAAAGCTTCTATGCCTGAATAGGTGACCGGAGGTCGGCACCTTTCCTTTGCAATT ACTGACCAC).

Further suitable internal ribosome entry sites include, but are not limited to, those described in Nucleic Acids Res. 2010 January;38 (Database issue): D131-6. doi: 10.1093/nar/gkp981. Epub 2009 Nov. 16, those described at iresite.org, those described in WO 2018/215787, the sequence described in GenBank accession No. KP019382.1, and the IRES element disclosed in GenBank accession No. LT727339.1.

Additional multi-cistronic linker regions, including cleavage, self-cleavage, and IRES elements, are disclosed in US 2018/0360992 and U.S. Pat. No. 8,865,467.

In some embodiments, the isolated nucleic acid encodes SEQ ID NO: 39 (MSVPTQVLGLLLLWLTDARCEIVMTQSPATLSVSPGERTTLSCRASQSVSSNLAWYLQ KPGQAPRLLIYGASTRATGIPARFSGSGSGTEFILTISSLQSEDFAVYYCQQYNNWPITFG QGTRLEIKGGGGSGGGGSGGGGEVOLVESGGGLVQPGRSLRLSCAASGFTFYDYAMH WVRQAPGKGLEWVSGISWNSGYIGYADSVKGRFTISRDNAKNSLYLQMNSLRAEDTAL YYCAKDNSYGKFYYGLDVWGQGTTVTVSSTTTPAPRPPTPAPTIASQPLSLRPEACRPA AGGAVHTRGLDFACDIYIWAPLAGTCGVLLLSLVITLYCQRRKYRSNKGESPVEPAEPC HYSCPREEEGSTIPIQEDYRKPEPACSPRVKFSRSADAPAYQQGQNQLYNELNLGRREEY DVLDKRRGRDPEMGGKPQRRKNPQEGLYNELQKDKMAEAYSEIGMKGERRRGKGHD GLYQGLSTATKDTYDALHMQALPPR), a 3H7-CD8-CD27z polypeptide comprising the following domains in order: a 3H7 binding domain, a CD8α hinge and transmembrane domain, a CD27 costimulation endodomain, and a CD3ζ signaling domain.

In some embodiments, the isolated nucleic acid encodes SEQ ID NO: 40 (MSVPTQVLGLLLLWLTDARCEIVMTQSPATLSVSPGERATLSCRASQSVSSNLAWYQQ KPGQAPRLLIYGTSTRATGIPARFSGSGSGTEFTLTISSLQSEDFAVYYCQQYNNWPLTFG GGTKVEIKGGGGSGGGGSGGGGEVOLVESGGGLVQPGRSLRLSCVASGFTFNDYAMH WVRQAPGKGLEWVSVISWNSDSIGYADSVKGRFTISRDNAKNSLYLQMHSLRAEDTAL YYCAKDNHYGSGSYYYYQYGMDVWGQGTTVTVSSTTTPAPRPPTPAPTIASQPLSLRPE ACRPAAGGAVHTRGLDFACDIYIWAPLAGTCGVLLLSLVITLYCKRGRKKLLYIFKQPF MRPVQTTQEEDGCSCRFPEEEEGGCELRVKFSRSADAPAYQQGQNQLYNELNLGRREE YDVLDKRRGRDPEMGGKPQRRKNPQEGLYNELQKDKMAEAYSEIGMKGERRRGKGH DGLYQGLSTATKDTYDALHMQALPPR), a 3B9-CD8-BBz polypeptide comprising the following domains in order: a 3B9 binding domain, a CD8α hinge and transmembrane domain, a 4-1BB costimulation endodomain, and a CD3ζ signaling domain.

In some embodiments, the isolated nucleic acid encodes SEQ ID NO: 41 (MSVPTQVLGLLLLWLTDARCEIVMTQSPATLSVSPGERTTLSCRASQSVSSNLAWYLQ KPGQAPRLLIYGASTRATGIPARFSGSGSGTEFILTISSLQSEDFAVYYCQQYNNWPITFG QGTRLEIKGGGGSGGGGSGGGGEVOLVESGGGLVQPGRSLRLSCAASGFTFYDYAMH WVRQAPGKGLEWVSGISWNSGYIGYADSVKGRFTISRDNAKNSLYLQMNSLRAEDTAL YYCAKDNSYGKFYYGLDVWGQGTTVTVSSTTTPAPRPPTPAPTIASQPLSLRPEACRPA AGGAVHTRGLDFACDIYIWAPLAGTCGVLLLSLVITLYCKRGRKKLLYIFKQPFMRPVQ TTQEEDGCCRFPEEEEGGCELRVKFSRSADAPAYQQGQNQLYNELNLGRREEYDVLD KRRGRDPEMGGKPQRRKNPQEGLYNELQKDKMAEAYSEIGMKGERRRGKGHDGLYQ GLSTATKDTYDALHMQALPPR), a 3H7-CD8-BBz polypeptide comprising the following domains in order: a 3H7 binding domain, a CD8α hinge and transmembrane domain, a 4-1BB costimulation endodomain, and a CD3ζ signaling domain.

In some embodiments, the isolated nucleic acid encodes SEQ ID NO: 42 (MSVPTQVLGLLLLWLTDARCEIVLTQSPATLSLSPGERAALSCRASQSVSNYLAWYQQ KPGQAPRLLIYDASNRATGIPARFSGSGSGTDFTLTISSLEPEDFAVYYCQQRSNWPLTFG GGTKVEIRGGGGSGGGGSGGGGEVOLVESGGGLVQPGRSLRLSCAASGFTFRDYTMH WVRQGPGKGLEWVSGISWNSDYIGYADSVKGRFTISRDNAKNSLYLQMNSLRVEDTAL YYCAKLSGTYRDYFYGVDVWGQGTTVTVSSTTTPAPRPPTPAPTIASQPLSLRPEACRPA AGGAVHTRGLDFACDIYIWAPLAGTCGVLLLSLVITLYCKRGRKKLLYIFKQPFMRPVQ TTQEEDGCSCRFPEEEEGGCELRVKFSRSADAPAYQQGQNQLYNELNLGRREEYDVLD KRRGRDPEMGGKPQRRKNPQEGLYNELQKDKMAEAYSEIGMKGERRRGKGHDGLYQ GLSTATKDTYDALHMQALPPR), a 2B7-CD8-BBz polypeptide comprising the following domains in order: a 2B7 binding domain, a CD8α hinge and transmembrane domain, a 4-1BB costimulation endodomain, and a CD3ζ signaling domain.

In some embodiments, the isolated nucleic acid encodes SEQ ID NO: 43 (MSVPTQVLGLLLLWLTDARCEIVVTQSPATLSLSPGERATLSCRTSQTTTSYLAWYRQK PGQAPRLLIYDASNRAAGIPARFSGSGSGTDFTLTINSLEPEDFAVYYCQLRTNWITFGQG TRLEIKGGGGSGGGGSGGGGQVQL VESGGDSVKPGGSLRLSCAASGFTFSDSYMTWIR QAPGKGLEWVSFISSSGSTIYYADSVKGRFTISRDNVKKSLYLQMNRLRAEDTAVYYCA REEPGNYVYYGMDVWGQGTTVTVSSTTTPAPRPPTPAPTIASQPLSLRPEACRPAAGGA VHTRGLDFACDIYIWAPLAGTCGVLLLSLVITLYCKRGRKKLLYIFKQPFMRPVQTTQEE DGCSCRFPEEEEGGCELRVKFSRSADAPAYQQGQNQLYNELNLGRREEYDVLDKRRGR DPEMGGKPQRRKNPQEGLYNELQKDKMAEAYSEIGMKGERRRGKGHDGLYQGLSTAT KDTYDALHMQALPPR), a 9C11-CD8-BBz polypeptide comprising the following domains in order: a 9C11 binding domain, a CD8α hinge and transmembrane domain, a 4-1BB costimulation endodomain, and a CD3ζ signaling domain.

In some embodiments, the isolated nucleic acid encodes SEQ ID NO: 44 (MSVPT QVLGLLLLWLTDARCEIVMTQSPATLSVSPGERTTLSCRASQSV SSNLAWYLQKPGQAPRLLIYGASTRATGIPARFSGSGSGTEFIL TISSLQSEDFAVYYCQQYNNWPITFGQGTRLEIKGGGGSGGGG SGGGGEVOLVESGGGLVQPGRSLRLSCAASGFTFYDYAMHWV RQAPGKGLEWVSGISWNSGYIGYADSVKGRFTISRDNAKNSLY LQMNSLRAEDTALYYCAKDNSYGKFYYGLDVWGQGTTVTVSS TTTPAPRPPTPAPTIASQPLSLRPEACRPAAGGAVHTRGLDFAC DIYIWAPLAGTCGVLLLSLVITLYCRVKFSRSADAPAY QQGQN QLYNELNLGRREEYDVLDKRRGRDPEMGGKPQRRKNPQEGLY NELQKDKMAEAYSEIGMKGERRRGKGHDGLYQGLSTATKDTY DALHMQALPPR), a 3H7-CD3z polypeptide comprising the following domains in order: a 3H7 binding domain, a CD8α hinge and transmembrane domain, and a CD3ζ signaling domain.

In some embodiments, the isolated nucleic acid encoding a 3H7-CD8-27z polypeptide comprises the sequence of SEQ ID NO: 45 (ATGTCCGTGCCTACCCAGGTGCTGGGCCTGCTGCTGCTGTGGCTGACCGACGCCAG ATGCGAAATAGTGATGACGCAGTCTCCAGCCACCCTGTCTGTGTCTCCAGGGGAAAG AACCACCCTCTCCTGCAGGGCCAGTCAGAGTGTTAGCAGCAACTTAGCCTGGTACCT TCAGAAACCTGGCCAGGCTCCCAGGCTCCTCATCTATGGTGCATCCACCAGGGCCAC TGGTATCCCAGCCAGGTTCAGTGGCAGTGGGTCTGGGACAGAGTTCATTCTCACCAT CAGCAGCCTGCAGTCTGAAGATTTTGCAGTTTATTACTGTCAGCAGTATAATAACTG GCCGATCACCTTCGGCCAAGGGACACGGCTGGAGATTAAAGGTGGAGGTGGATCTG GAGGAGGAGGATCCGGTGGAGGAGGTGAAGTGCAACTGGTGGAGTCTGGGGGAGG CTTGGTACAGCCTGGCAGGTCCCTGAGACTCTCCTGTGCAGCCTCTGGATTCACCTTT TATGATTATGCCATGCACTGGGTCCGGCAAGCTCCAGGGAAGGGCCTGGAGTGGGT CTCAGGTATTAGTTGGAATAGTGGTTACATAGGCTATGCGGACTCTGTGAAGGGCCG ATTCACCATCTCCAGAGACAACGCCAAGAACTCCCTGTATCTGCAAATGAACAGTCT GAGAGCTGAGGACACGGCCTTGTATTACTGTGCAAAAGATAACAGCTATGGAAAGT TCTACTACGGTTTGGACGTCTGGGGCCAAGGGACCACGGTCACCGTCTCCTCAACCA CGACGCCAGCGCCGCGACCACCAACACCGGCGCCCACCATCGCGTCGCAGCCCCTG TCCCTGCGCCCAGAGGCGTGCCGGCCAGCGGCGGGGGGCGCAGTGCACACGAGGGG GCTGGACTTCGCCTGTGATATCTACATCTGGGCGCCCTTGGCCGGGACTTGTGGGGT CCTTCTCCTGTCACTGGTTATCACCCTTTACTGCCAACGACGCAAGTACCGCTCCAAT AAAGGAGAGTCACCAGTAGAACCCGCCGAACCTTGTCACTATTCATGTCCACGCGA AGAGGAGGGTTCAACGATCCCTATTCAGGAAGATTACAGAAAGCCGGAACCTGCTT GTAGCCCCAGAGTGAAGTTCAGCCGCAGCGCCGACGCCCCTGCCTACCAGCAGGGC CAGAACCAGCTGTATAACGAGCTGAACCTGGGCAGGCGGGAGGAATACGACGTGCT GGACAAGCGCAGAGGCCGGGACCCTGAGATGGGCGGCAAGCCCCAGAGGCGGAAG AACCCCCAGGAAGGCCTGTATAACGAACTGCAGAAAGACAAGATGGCCGAGGCCTA CAGCGAGATCGGCATGAAGGGCGAGCGGCGACGCGGCAAGGGCCACGACGGCCTG TACCAGGGCCTGTCCACCGCCACCAAGGACACCTACGACGCCCTGCACATGCAGGC CCTGCCTCCCCGTTAG).

In some embodiments, the isolated nucleic acid encoding a 3H7-CD8-BBz polypeptide comprises the sequence of 46 SEQ ID NO: (ATGTCCGTGCCTACCCAGGTGCTGGGCCTGCTGCTGCTGTGGCTGACCGACGCCAG ATGCGAAATAGTGATGACGCAGTCTCCAGCCACCCTGTCTGTGTCTCCAGGGGAAAG AACCACCCTCTCCTGCAGGGCCAGTCAGAGTGTTAGCAGCAACTTAGCCTGGTACCT TCAGAAACCTGGCCAGGCTCCCAGGCTCCTCATCTATGGTGCATCCACCAGGGCCAC TGGTATCCCAGCCAGGTTCAGTGGCAGTGGGTCTGGGACAGAGTTCATTCTCACCAT CAGCAGCCTGCAGTCTGAAGATTTTGCAGTTTATTACTGTCAGCAGTATAATAACTG GCCGATCACCTTCGGCCAAGGGACACGGCTGGAGATTAAAGGTGGAGGTGGATCTG GAGGAGGAGGATCCGGTGGAGGAGGTGAAGTGCAACTGGTGGAGTCTGGGGGAGG CTTGGTACAGCCTGGCAGGTCCCTGAGACTCTCCTGTGCAGCCTCTGGATTCACCTTT TATGATTATGCCATGCACTGGGTCCGGCAAGCTCCAGGGAAGGGCCTGGAGTGGGT CTCAGGTATTAGTTGGAATAGTGGTTACATAGGCTATGCGGACTCTGTGAAGGGCCG ATTCACCATCTCCAGAGACAACGCCAAGAACTCCCTGTATCTGCAAATGAACAGTCT GAGAGCTGAGGACACGGCCTTGTATTACTGTGCAAAAGATAACAGCTATGGAAAGT TCTACTACGGTTTGGACGTCTGGGGCCAAGGGACCACGGTCACCGTCTCCTCAACCA CGACGCCAGCGCCGCGACCACCAACACCGGCGCCCACCATCGCGTCGCAGCCCCTG TCCCTGCGCCCAGAGGCGTGCCGGCCAGCGGCGGGGGGCGCAGTGCACACGAGGGG GCTGGACTTCGCCTGTGATATCTACATCTGGGCGCCCTTGGCCGGGACTTGTGGGGT CCTTCTCCTGTCACTGGTTATCACCCTTTACTGCAAACGGGGCAGAAAGAAACTCCT GTATATATTCAAACAACCATTTATGAGACCAGTACAAACTACTCAAGAGGAAGATG GCTGTAGCTGCCGATTTCCAGAAGAAGAAGAAGGAGGATGTGAACTGAGAGTGAAG TTCAGCAGGAGCGCAGACGCCCCCGCGTACCAGCAGGGCCAGAACCAGCTCTATAA CGAGCTCAATCTAGGACGAAGAGAGGAGTACGATGTTTTGGACAAGAGACGTGGCC GGGACCCTGAGATGGGGGGAAAGCCGCAGAGAAGGAAGAACCCTCAGGAAGGCCT GTACAATGAACTGCAGAAAGATAAGATGGCGGAGGCCTACAGTGAGATTGGGATGA AAGGCGAGCGCCGGAGGGGCAAGGGGCACGATGGCCTTTACCAGGGTCTCAGTACA GCCACCAAGGACACCTACGACGCCCTTCACATGCAGGCCCTGCCCCCTCGCTAA).

In some embodiments, the isolated nucleic acid encoding a 3B9-CD8-BBz polypeptide comprises the sequence of SEQ ID NO: 47 (ATGAGCGTTCCAACCCAAGTTCTGGGACTGCTTCTGCTCTGGTTGACTGACGCTAGG TGCGAAATAGTAATGACCCAATCCCCAGCCACTCTCTCCGTTAGCCCAGGTGAAAGA GCCACTCTTAGTTGCAGGGCTAGTCAATCCGTATCTAGCAACCTGGCCTGGTACCAG CAAAAGCCCGGACAAGCGCCGCGGTTGTTGATCTATGGGACGAGCACACGAGCTAC GGGTATTCCGGCCAGGTTCTCAGGGTCTGGCTCCGGAACCGAATTTACATTGACGAT CAGTAGTCTGCAATCAGAGGATTTCGCCGTTTACTATTGCCAACAGTACAATAATTG GCCGCTCACATTCGGGGGAGGAACCAAGGTCGAGATTAAGGGAGGTGGGGGTAGTG GGGGCGGGGGGTCAGGAGGTGGAGGAGAGGTACAGTTGGTAGAAAGCGGCGGGGG GTTGGTTCAACCTGGACGGAGTCTGAGATTGTCTTGCGTGGCTTCCGGCTTTACTTTC AATGATTACGCCATGCACTGGGTACGCCAGGCGCCTGGAAAGGGTCTGGAGTGGGT TTCCGTGATATCCTGGAATAGTGATAGTATAGGCTATGCCGATAGTGTAAAAGGAAG GTTTACAATCTCTAGGGATAACGCTAAGAACAGCCTGTACCTTCAAATGCATAGTCT CCGGGCTGAGGACACAGCCTTGTACTATTGTGCTAAGGACAATCATTATGGAAGCG GGTCATATTATTACTATCAATATGGGATGGATGTGTGGGGTCAGGGAACGACCGTTA CGGTATCCTCAACCACCACCCCTGCACCAAGGCCCCCGACTCCCGCGCCCACCATCG CGTCACAGCCTCTTAGCCTGCGACCGGAAGCATGCAGACCAGCTGCCGGGGGGGCC GTGCATACGAGAGGTTTGGACTTCGCCTGCGATATCTACATCTGGGCGCCCTTGGCC GGGACTTGTGGGGTCCTTCTCCTGTCACTGGTTATCACCCTTTACTGCAAACGGGGC AGAAAGAAACTCCTGTATATATTCAAACAACCATTTATGAGACCAGTACAAACTACT CAAGAGGAAGATGGCTGTAGCTGCCGATTTCCAGAAGAAGAAGAAGGAGGATGTG AACTGAGAGTGAAGTTCAGCAGGAGCGCAGACGCCCCCGCGTACCAGCAGGGCCAG AACCAGCTCTATAACGAGCTCAATCTAGGACGAAGAGAGGAGTACGATGTTTTGGA CAAGAGACGTGGCCGGGACCCTGAGATGGGGGGAAAGCCGCAGAGAAGGAAGAAC CCTCAGGAAGGCCTGTACAATGAACTGCAGAAAGATAAGATGGCGGAGGCCTACAG TGAGATTGGGATGAAAGGCGAGCGCCGGAGGGGCAAGGGGCACGATGGCCTTTACC AGGGTCTCAGTACAGCCACCAAGGACACCTACGACGCCCTTCACATGCAGGCCCTG CCCCCTCGCTAA).

In some embodiments, the isolated nucleic acid encoding a 2B7-CD8-BBz polypeptide comprises the sequence of SEQ ID NO: 48 (ATGTCCGTACCTACCCAGGTGCTGGGCCTGCTGCTGCTGTGGCTGACCGACGCCAG ATGCGAAATTGTGTTGACACAGTCTCCAGCCACCCTGTCTTTGTCTCCAGGGGAAAG AGCCGCCCTCTCCTGCAGGGCCAGTCAGAGTGTTAGCAACTACTTAGCCTGGTACCA ACAGAAACCTGGCCAGGCTCCCAGGCTCCTCATCTATGATGCATCCAACAGGGCCA CTGGCATCCCAGCCAGGTTCAGTGGCAGTGGGTCTGGGACAGACTTCACTCTCACCA TCAGCAGCCTAGAGCCTGAAGATTTTGCAGTTTATTACTGTCAGCAGCGTAGCAACT GGCCGCTCACTTTCGGCGGAGGGACCAAGGTGGAGATCAGAGGTGGAGGTGGATCT GGAGGAGGAGGATCCGGTGGAGGAGGTGAAGTGCAGCTGGTGGAGTCTGGGGGAG GCTTGGTACAGCCTGGCAGGTCCCTGCGACTCTCCTGTGCAGCCTCTGGATTCACCT TTCGAGATTATACCATGCACTGGGTCCGGCAAGGTCCAGGGAAGGGCCTGGAATGG GTCTCAGGTATTAGTTGGAATAGTGATTACATAGGCTATGCGGACTCTGTGAAGGGC CGATTCACCATCTCCAGAGACAACGCCAAGAACTCCCTGTATCTGCAAATGAACAGT CTGAGAGTTGAGGACACGGCCTTGTATTACTGTGCAAAGCTCAGTGGGACCTACAG GGACTACTTCTACGGAGTGGACGTCTGGGGCCAAGGGACCACGGTCACCGTCTCCTC AACCACCACCCCTGCACCAAGGCCCCCGACTCCCGCGCCCACCATCGCGTCACAGCC TCTTAGCCTGCGACCGGAAGCATGCAGACCAGCTGCCGGGGGGGCCGTGCATACGA GAGGTTTGGACTTCGCCTGCGATATCTACATCTGGGCGCCCTTGGCCGGGACTTGTG GGGTCCTTCTCCTGTCACTGGTTATCACCCTTTACTGCAAACGGGGCAGAAAGAAAC TCCTGTATATATTCAAACAACCATTTATGAGACCAGTACAAACTACTCAAGAGGAAG ATGGCTGTAGCTGCCGATTTCCAGAAGAAGAAGAAGGAGGATGTGAACTGAGAGTG AAGTTCAGCAGGAGCGCAGACGCCCCCGCGTACCAGCAGGGCCAGAACCAGCTCTA TAACGAGCTCAATCTAGGACGAAGAGAGGAGTACGATGTTTTGGACAAGAGACGTG GCCGGGACCCTGAGATGGGGGGAAAGCCGCAGAGAAGGAAGAACCCTCAGGAAGG CCTGTACAATGAACTGCAGAAAGATAAGATGGCGGAGGCCTACAGTGAGATTGGGA TGAAAGGCGAGCGCCGGAGGGGCAAGGGGCACGATGGCCTTTACCAGGGTCTCAGT ACAGCCACCAAGGACACCTACGACGCCCTTCACATGCAGGCCCTGCCCCCTCGCTAA).

In some embodiments, the isolated nucleic acid encoding a 9C11-CD8-BBz polypeptide comprises the sequence of SEQ ID NO: 49 (ATGTCCGTGCCTACCCAGGTGCTGGGCCTGCTGCTGCTGTGGCTGACCGACGCCAG ATGCGAAATTGTGGTGACACAGTCTCCAGCCACCCTGTCTTTGTCTCCAGGGGAAAG AGCCACCCTCTCCTGCAGGACCAGTCAGACTACTACCAGCTACTTAGCCTGGTACCG ACAGAAACCTGGCCAGGCTCCCAGGCTCCTCATCTATGATGCATCCAACAGGGCCG CTGGCATCCCAGCCAGGTTCAGTGGCAGTGGGTCTGGGACAGACTTCACTCTCACCA TCAACAGCCTGGAGCCTGAAGATTTTGCAGTTTATTACTGTCAGCTGCGTACCAACT GGATCACCTTCGGCCAAGGGACACGACTGGAGATTAAAGGTGGAGGTGGATCTGGA GGAGGAGGATCCGGTGGAGGAGGTCAGGTGCAGCTGGTGGAGTCTGGGGGAGACTC GGTCAAGCCTGGAGGGTCCCTGAGACTCTCCTGTGCAGCCTCTGGATTCACCTTCAG TGACTCCTACATGACTTGGATCCGCCAGGCTCCAGGGAAGGGGCTGGAGTGGGTTTC ATTCATTAGTAGTAGTGGAAGTACCATATATTATGCAGACTCTGTGAAGGGCCGATT CACCATTTCCAGGGACAACGTCAAGAAGTCATTGTATCTGCAGATGAACAGACTGA GAGCCGAGGACACGGCCGTGTATTACTGTGCGAGAGAAGAACCAGGAAACTACGTC TATTACGGTATGGACGTCTGGGGCCAAGGGACCACGGTCACCGTCTCCTCAACCACC ACCCCTGCACCAAGGCCCCCGACTCCCGCGCCCACCATCGCGTCACAGCCTCTTAGC CTGCGACCGGAAGCATGCAGACCAGCTGCCGGGGGGGCCGTGCATACGAGAGGTTT GGACTTCGCCTGCGATATCTACATCTGGGCGCCCTTGGCCGGGACTTGTGGGGTCCT TCTCCTGTCACTGGTTATCACCCTTTACTGCAAACGGGGCAGAAAGAAACTCCTGTA TATATTCAAACAACCATTTATGAGACCAGTACAAACTACTCAAGAGGAAGATGGCT GTAGCTGCCGATTTCCAGAAGAAGAAGAAGGAGGATGTGAACTGAGAGTGAAGTTC AGCAGGAGCGCAGACGCCCCCGCGTACCAGCAGGGCCAGAACCAGCTCTATAACGA GCTCAATCTAGGACGAAGAGAGGAGTACGATGTTTTGGACAAGAGACGTGGCCGGG ACCCTGAGATGGGGGGAAAGCCGCAGAGAAGGAAGAACCCTCAGGAAGGCCTGTA CAATGAACTGCAGAAAGATAAGATGGCGGAGGCCTACAGTGAGATTGGGATGAAA GGCGAGCGCCGGAGGGGCAAGGGGCACGATGGCCTTTACCAGGGTCTCAGTACAGC CACCAAGGACACCTACGACGCCCTTCACATGCAGGCCCTGCCCCCTCGCTAA).

In some embodiments, the isolated nucleic acid comprises a codon optimized sequence encoding a CD8α hinge region. Exemplary codon optimized CD8α hinge region nucleic acid sequences include, without limitation, SEQ ID NO: 50 (ACCACCACCCCTGCACCAAGGCCCCCGACTCCCGCGCCCACCATCGCGTCA CAGCCTCTTAGCCTGCGACCGGAAGCATGCAGACCAGCTGCCGGGGGGGCCGTGCA TACGAGAGGTTTGGACTTCGCCTGCGAT). In some embodiments, the CD8α hinge region is encoded by the following sequence SEQ ID NO: 51 (ACCACGACGCCAGCGCCGCGACCACCAACACCGGCGCCCACCATCGCGTCGCAGCC CCTGTCCCTGCGCCCAGAGGCGTGCCGGCCAGCGGCGGGGGGCGCAGTGCACACGA GGGGGCTGGACTTCGCCTGTGAT).

In some embodiments, the isolated nucleic acid encodes a 3B9 binding domain and comprises the following sequence encoding a CD8α hinge domain SEQ ID NO: 50. In some embodiments, the isolated nucleic acid encodes a 2B7 binding domain and comprises the following sequence encoding a CD8α hinge domain SEQ ID NO: 50. In some embodiments, the isolated nucleic acid encodes a 9C11 binding domain and comprises the following sequence encoding a CD8α hinge domain SEQ ID NO: 50. In some embodiments, the isolated nucleic acid encodes a 3H7 binding domain and comprises the following sequence encoding a CD8α hinge domain SEQ ID NO: 51.

In some embodiments, the isolated nucleic acid encodes SEQ ID NO: 52 (MSVPTQVLGLLLLWLTDARCEIVMTQSPATLSVSPGERTTLSCRASQSVSSNLAWYLQ KPGQAPRLLIYGASTRATGIPARFSGSGSGTEFILTISSLQSEDFAVYYCQQYNNWPITFG QGTRLEIKGGGGSGGGGSGGGGEVOLVESGGGLVQPGRSLRLSCAASGFTFYDYAMH WVRQAPGKGLEWVSGISWNSGYIGYADSVKGRFTISRDNAKNSLYLQMNSLRAEDTAL YYCAKDNSYGKFYYGLDVWGQGTTVTVSSTTTPAPRPPTPAPTIASQPLSLRPEACRPA AGGAVHTRGLDFACDIYIWAPLAGTCGVLLLSLVITLYCKRGRKKLLYIFKQPFMRPVQ TTQEEDGCSCRFPEEEEGGCELRVKFSRSADAPAYQQGQNQLYNELNLGRREEYDVLD KRRGRDPEMGGKPQRRKNPQEGLYNELQKDKMAEAYSEIGMKGERRRGKGHDGLYQ GLSTATKDTYDALHMQALPPRGSGATNFSLLKQAGDVEENPGPMALPVTALLLPLALLL HAARPNWVNVISDLKKIEDLIQSMHIDATLYTESDVHPSCKVTAMKCFLLELQVISLESG DASIHDTVENLIILANNSLSSNGNVTESGCKECEELEEKNIKEFLQSFVHIVQMFINTS*), an anti-CD20-CAR polypeptide comprising the following domains in order: a 3H7 binding domain, a CD8a hinge and transmembrane domain, a 4-1BB costimulation endodomain, a CD3ζ signaling domain, a P2A cleavage domain (GSGATNFSLLKQAGDVEENPGP, SEQ ID NO: 53), a secretion signal, and a sIL 15 domain.

In some embodiments, the isolated nucleic acid encodes SEQ ID NO: 54 (MSVPTQVLGLLLLWLTDARCEIVMTQSPATLSVSPGERTTLSCRASQSVSSNLAWYLQ KPGQAPRLLIYGASTRATGIPARFSGSGSGTEFILTISSLQSEDFAVYYCQQYNNWPITFG QGTRLEIKGGGGSGGGGSGGGGEVOL VESGGGLVQPGRSLRLSCAASGFTFYDYAMH WVRQAPGKGLEWVSGISWNSGYIGYADSVKGRFTISRDNAKNSLYLQMNSLRAEDTAL YYCAKDNSYGKFYYGLDVWGQGTTVTVSSTTTPAPRPPTPAPTIASQPLSLRPEACRPA AGGAVHTRGLDFACDIYIWAPLAGTCGVLLLSL VITLYCKRGRKKLLYIFKQPFMRPVQ TTQEEDGCSCRFPEEEEGGCELRVKFSRSADAPAYQQGQNQLYNELNLGRREEYDVLD KRRGRDPEMGGKPQRRKNPQEGLYNELQKDKMAEAYSEIGMKGERRRGKGHDGLYQ GLSTATKDTYDALHMQALPPRGSGATNFSLLKQAGDVEENPGPMRISKPHLRSISIQCYL CLLLNSHFLTEAGIHVFILGCFSAGLPKTEANWVNVISDLKKIEDLIQSMHIDATLYTESD VHPSCKVTAMKCFLLELQVISLESGDASIHDTVENLIILANNSLSSNGNVTESGCKECEEL EEKNIKEFLQSFVHIVQMFINTS*), an anti-CD20-CAR polypeptide comprising the following domains in order: a 3H7 binding domain, a CD8α hinge and transmembrane domain, a 4-1BB costimulation endodomain, a CD3ζ signaling domain, a P2A cleavage domain of SEQ ID NO: 53, a secretion signal of SEQ ID NO: 55 (MRISKPHLRSISIQCYLCLLLNSHFLTEAG IHVFILGCFSAGLPKTEA), and a sIL 15 domain.

In some embodiments, the isolated nucleic acid encoding an anti-CD20 CAR+sIL 15 polypeptide comprises the sequence of SEQ ID NO: 56 (ATGTCCGTGCCTACCCAGGTGCTGGGCCTGCTGCTGCTGTGGCTGACCGACGCCAG ATGCGAAATAGTGATGACGCAGTCTCCAGCCACCCTGTCTGTGTCTCCAGGGGAAAG AACCACCCTCTCCTGCAGGGCCAGTCAGAGTGTTAGCAGCAACTTAGCCTGGTACCT TCAGAAACCTGGCCAGGCTCCCAGGCTCCTCATCTATGGTGCATCCACCAGGGCCAC TGGTATCCCAGCCAGGTTCAGTGGCAGTGGGTCTGGGACAGAGTTCATTCTCACCAT CAGCAGCCTGCAGTCTGAAGATTTTGCAGTTTATTACTGTCAGCAGTATAATAACTG GCCGATCACCTTCGGCCAAGGGACACGGCTGGAGATTAAAGGTGGAGGTGGATCTG GAGGAGGAGGATCCGGTGGAGGAGGTGAAGTGCAACTGGTGGAGTCTGGGGGAGG CTTGGTACAGCCTGGCAGGTCCCTGAGACTCTCCTGTGCAGCCTCTGGATTCACCTTT TATGATTATGCCATGCACTGGGTCCGGCAAGCTCCAGGGAAGGGCCTGGAGTGGGT CTCAGGTATTAGTTGGAATAGTGGTTACATAGGCTATGCGGACTCTGTGAAGGGCCG ATTCACCATCTCCAGAGACAACGCCAAGAACTCCCTGTATCTGCAAATGAACAGTCT GAGAGCTGAGGACACGGCCTTGTATTACTGTGCAAAAGATAACAGCTATGGAAAGT TCTACTACGGTTTGGACGTCTGGGGCCAAGGGACCACGGTCACCGTCTCCTCAACCA CGACGCCAGCGCCGCGACCACCAACACCGGCGCCCACCATCGCGTCGCAGCCCCTG TCCCTGCGCCCAGAGGCGTGCCGGCCAGCGGCGGGGGGCGCAGTGCACACGAGGGG GCTGGACTTCGCCTGTGATATCTACATCTGGGCGCCCTTGGCCGGGACTTGTGGGGT CCTTCTCCTGTCACTGGTTATCACCCTTTACTGCAAACGGGGCAGAAAGAAACTCCT GTATATATTCAAACAACCATTTATGAGACCAGTACAAACTACTCAAGAGGAAGATG GCTGTAGCTGCCGATTTCCAGAAGAAGAAGAAGGAGGATGTGAACTGAGAGTGAAG TTCAGCAGGAGCGCAGACGCCCCCGCGTACCAGCAGGGCCAGAACCAGCTCTATAA CGAGCTCAATCTAGGACGAAGAGAGGAGTACGATGTTTTGGACAAGAGACGTGGCC GGGACCCTGAGATGGGGGGAAAGCCGCAGAGAAGGAAGAACCCTCAGGAAGGCCT GTACAATGAACTGCAGAAAGATAAGATGGCGGAGGCCTACAGTGAGATTGGGATGA AAGGCGAGCGCCGGAGGGGCAAGGGGCACGATGGCCTTTACCAGGGTCTCAGTACA GCCACCAAGGACACCTACGACGCCCTTCACATGCAGGCCCTGCCCCCTCGCGGTAGC GGGGCTACGAACTTCTCCCTTCTTAAACAAGCGGGAGACGTGGAAGAAAATCCCGG ACCTATGGCCTTACCAGTGACCGCCTTGCTCCTGCCGCTGGCCTTGCTGCTCCACGCC GCCAGGCCGAACTGGGTGAATGTAATAAGTGATTTGAAAAAAATTGAAGATCTTAT TCAATCTATGCATATTGATGCTACTTTATATACGGAAAGTGATGTTCACCCCAGTTGC AAAGTAACAGCAATGAAGTGCTTTCTCTTGGAGTTACAAGTTATTTCACTTGAGTCC GGAGATGCAAGTATTCATGATACAGTAGAAAATCTGATCATCCTAGCAAACAACAG TTTGTCTTCTAATGGGAATGTAACAGAATCTGGATGCAAAGAATGTGAGGAACTGG AGGAAAAAAATATTAAAGAATTTTTGCAGAGTTTTGTACATATTGTCCAAATGTTCA TCAACACTTCTTGA).

In some embodiments, the isolated nucleic acid encoding an anti-CD20 CAR+sIL 15 polypeptide comprises the sequence of SEQ ID NO: 57 (ATGTCCGTGCCTACCCAGGTGCTGGGCCTGCTGCTGCTGTGGCTGACCGACGCCAG ATGCGAAATAGTGATGACGCAGTCTCCAGCCACCCTGTCTGTGTCTCCAGGGGAAAG AACCACCCTCTCCTGCAGGGCCAGTCAGAGTGTTAGCAGCAACTTAGCCTGGTACCT TCAGAAACCTGGCCAGGCTCCCAGGCTCCTCATCTATGGTGCATCCACCAGGGCCAC TGGTATCCCAGCCAGGTTCAGTGGCAGTGGGTCTGGGACAGAGTTCATTCTCACCAT CAGCAGCCTGCAGTCTGAAGATTTTGCAGTTTATTACTGTCAGCAGTATAATAACTG GCCGATCACCTTCGGCCAAGGGACACGGCTGGAGATTAAAGGTGGAGGTGGATCTG GAGGAGGAGGATCCGGTGGAGGAGGTGAAGTGCAACTGGTGGAGTCTGGGGGAGG CTTGGTACAGCCTGGCAGGTCCCTGAGACTCTCCTGTGCAGCCTCTGGATTCACCTTT TATGATTATGCCATGCACTGGGTCCGGCAAGCTCCAGGGAAGGGCCTGGAGTGGGT CTCAGGTATTAGTTGGAATAGTGGTTACATAGGCTATGCGGACTCTGTGAAGGGCCG ATTCACCATCTCCAGAGACAACGCCAAGAACTCCCTGTATCTGCAAATGAACAGTCT GAGAGCTGAGGACACGGCCTTGTATTACTGTGCAAAAGATAACAGCTATGGAAAGT TCTACTACGGTTTGGACGTCTGGGGCCAAGGGACCACGGTCACCGTCTCCTCAACCA CGACGCCAGCGCCGCGACCACCAACACCGGCGCCCACCATCGCGTCGCAGCCCCTG TCCCTGCGCCCAGAGGCGTGCCGGCCAGCGGCGGGGGGCGCAGTGCACACGAGGGG GCTGGACTTCGCCTGTGATATCTACATCTGGGCGCCCTTGGCCGGGACTTGTGGGGT CCTTCTCCTGTCACTGGTTATCACCCTTTACTGCAAACGGGGCAGAAAGAAACTCCT GTATATATTCAAACAACCATTTATGAGACCAGTACAAACTACTCAAGAGGAAGATG GCTGTAGCTGCCGATTTCCAGAAGAAGAAGAAGGAGGATGTGAACTGAGAGTGAAG TTCAGCAGGAGCGCAGACGCCCCCGCGTACCAGCAGGGCCAGAACCAGCTCTATAA CGAGCTCAATCTAGGACGAAGAGAGGAGTACGATGTTTTGGACAAGAGACGTGGCC GGGACCCTGAGATGGGGGGAAAGCCGCAGAGAAGGAAGAACCCTCAGGAAGGCCT GTACAATGAACTGCAGAAAGATAAGATGGCGGAGGCCTACAGTGAGATTGGGATGA AAGGCGAGCGCCGGAGGGGCAAGGGGCACGATGGCCTTTACCAGGGTCTCAGTACA GCCACCAAGGACACCTACGACGCCCTTCACATGCAGGCCCTGCCCCCTCGCGGTAGC GGGGCTACGAACTTCTCCCTTCTTAAACAAGCGGGAGACGTGGAAGAAAATCCCGG ACCTATGAGAATTTCGAAACCACATTTGAGAAGTATTTCCATCCAGTGCTACTTGTG TTTACTTCTAAACAGTCATTTTCTAACTGAAGCTGGCATTCATGTCTTCATTTTGGGC TGTTTCAGTGCAGGGCTTCCTAAAACAGAAGCCAACTGGGTGAATGTAATAAGTGAT TTGAAAAAAATTGAAGATCTTATTCAATCTATGCATATTGATGCTACTTTATATACG GAAAGTGATGTTCACCCCAGTTGCAAAGTAACAGCAATGAAGTGCTTTCTCTTGGAG TTACAAGTTATTTCACTTGAGTCCGGAGATGCAAGTATTCATGATACAGTAGAAAAT CTGATCATCCTAGCAAACAACAGTTTGTCTTCTAATGGGAATGTAACAGAATCTGGA TGCAAAGAATGTGAGGAACTGGAGGAAAAAAATATTAAAGAATTTTTGCAGAGTTT TGTACATATTGTCCAAATGTTCATCAACACTTCTTGA).

In some embodiments, the isolated nucleic acid encodes SEQ ID NO: 58 (MSVPTQVLGLLLLWLTDARCEIVMTQSPATLSVSPGERTTLSCRASQSVSSNLAWYLQ KPGQAPRLLIYGASTRATGIPARFSGSGSGTEFILTISSLQSEDFAVYYCQQYNNWPITFG QGTRLEIKGGGGSGGGGSGGGGEVOLVESGGGLVQPGRSLRLSCAASGFTFYDYAMH WVRQAPGKGLEWVSGISWNSGYIGYADSVKGRFTISRDNAKNSLYLQMNSLRAEDTAL YYCAKDNSYGKFYYGLDVWGQGTTVTVSSTTTPAPRPPTPAPTIASQPLSLRPEACRPA AGGAVHTRGLDFACDIYIWAPLAGTCGVLLLSLVITLYCKRGRKKLLYIFKQPFMRPVQ TTQEEDGCSCRFPEEEEGGCELRVKFSRSADAPAYQQGQNQLYNELNLGRREEYDVLD KRRGRDPEMGGKPQRRKNPQEGLYNELQKDKMAEAYSEIGMKGERRRGKGHDGLYQ GLSTATKDTYDALHMQALPPR*), an anti-CD20-CAR polypeptide comprising the following domains in order: a 3H7 binding domain, a CD8α hinge and transmembrane domain, a 4-1BB costimulation endodomain, and a CD3ζ signaling domain; and, via an internal ribosome entry site (e.g., encoded by SEQ ID NO: 37) 3′of the region encoding SEQ ID NO: 58, the isolated nucleic acid further encodes SEQ ID NO: 59 (MALPVTALLLPLALLLHAARPNWVNVISDLKKIEDLIQSMHIDATLYTESDVHP SCKVTAMKCFLLELQVISLESGDASIHDTVENLIILANNSLSSNGNVTESGCKECEELEEK NIKEFLQSFVHIVQMFINTS*), a secretion signal of SEQ ID NO: 28 and a sIL15 domain.

In some embodiments, the isolated nucleic acid encoding an anti-CD20 CAR+sIL 15 polypeptide comprises the sequence of SEQ ID NO: 60 (ATGTCCGTGCCTACCCAGGTGCTGGGCCTGCTGCTGCTGTGGCTGACCGACGCCAG ATGCGAAATAGTGATGACGCAGTCTCCAGCCACCCTGTCTGTGTCTCCAGGGGAAAG AACCACCCTCTCCTGCAGGGCCAGTCAGAGTGTTAGCAGCAACTTAGCCTGGTACCT TCAGAAACCTGGCCAGGCTCCCAGGCTCCTCATCTATGGTGCATCCACCAGGGCCAC TGGTATCCCAGCCAGGTTCAGTGGCAGTGGGTCTGGGACAGAGTTCATTCTCACCAT CAGCAGCCTGCAGTCTGAAGATTTTGCAGTTTATTACTGTCAGCAGTATAATAACTG GCCGATCACCTTCGGCCAAGGGACACGGCTGGAGATTAAAGGTGGAGGTGGATCTG GAGGAGGAGGATCCGGTGGAGGAGGTGAAGTGCAACTGGTGGAGTCTGGGGGAGG CTTGGTACAGCCTGGCAGGTCCCTGAGACTCTCCTGTGCAGCCTCTGGATTCACCTTT TATGATTATGCCATGCACTGGGTCCGGCAAGCTCCAGGGAAGGGCCTGGAGTGGGT CTCAGGTATTAGTTGGAATAGTGGTTACATAGGCTATGCGGACTCTGTGAAGGGCCG ATTCACCATCTCCAGAGACAACGCCAAGAACTCCCTGTATCTGCAAATGAACAGTCT GAGAGCTGAGGACACGGCCTTGTATTACTGTGCAAAAGATAACAGCTATGGAAAGT TCTACTACGGTTTGGACGTCTGGGGCCAAGGGACCACGGTCACCGTCTCCTCAACCA CGACGCCAGCGCCGCGACCACCAACACCGGCGCCCACCATCGCGTCGCAGCCCCTG TCCCTGCGCCCAGAGGCGTGCCGGCCAGCGGCGGGGGGCGCAGTGCACACGAGGGG GCTGGACTTCGCCTGTGATATCTACATCTGGGCGCCCTTGGCCGGGACTTGTGGGGT CCTTCTCCTGTCACTGGTTATCACCCTTTACTGCAAACGGGGCAGAAAGAAACTCCT GTATATATTCAAACAACCATTTATGAGACCAGTACAAACTACTCAAGAGGAAGATG GCTGTAGCTGCCGATTTCCAGAAGAAGAAGAAGGAGGATGTGAACTGAGAGTGAAG TTCAGCAGGAGCGCAGACGCCCCCGCGTACCAGCAGGGCCAGAACCAGCTCTATAA CGAGCTCAATCTAGGACGAAGAGAGGAGTACGATGTTTTGGACAAGAGACGTGGCC GGGACCCTGAGATGGGGGGAAAGCCGCAGAGAAGGAAGAACCCTCAGGAAGGCCT GTACAATGAACTGCAGAAAGATAAGATGGCGGAGGCCTACAGTGAGATTGGGATGA AAGGCGAGCGCCGGAGGGGCAAGGGGCACGATGGCCTTTACCAGGGTCTCAGTACA GCCACCAAGGACACCTACGACGCCCTTCACATGCAGGCCCTGCCCCCTCGCTAGAGT ACTGCGGCCGCTACGTAAATTCCGCCCCTCTCCCTCCCCCCCCCCTAACGTTACTGGC CGAAGCCGCTTGGAATAAGGCCGGTGTGCGTTTGTCTATATGTTATTTTCCACCATAT TGCCGTCTTTTGGCAATGTGAGGGCCCGGAAACCTGGCCCTGTCTTCTTGACGAGCA TTCCTAGGGGTCTTTCCCCTCTCGCCAAAGGAATGCAAGGTCTGTTGAATGTCGTGA AGGAAGCAGTTCCTCTGGAAGCTTCTTGAAGACAAACAACGTCTGTAGCGACCCTTT GCAGGCAGCGGAACCCCCCACCTGGCGACAGGTGCCTCTGCGGCCAAAAGCCACGT GTATAAGATACACCTGCAAAGGCGGCACAACCCCAGTGCCACGTTGTGAGTTGGAT AGTTGTGGAAAGAGTCAAATGGCTCTCCTCAAGCGTATTCAACAAGGGGCTGAAGG ATGCCCAGAAGGTACCCCATTGTATGGGATCTGATCTGGGGCCTCGGTGCACATGCT TTACATGTGTTTAGTCGAGGTTAAAAAAACGTCTAGGCCCCCCGAACCACGGGGAC GTGGTTTTCCTTTGAAAAACACGATGATATTAATTAAGCCACCGCCATGGCCTTACC AGTGACCGCCTTGCTCCTGCCGCTGGCCTTGCTGCTCCACGCCGCCAGGCCGAACTG GGTGAATGTAATAAGTGATTTGAAAAAAATTGAAGATCTTATTCAATCTATGCATAT TGATGCTACTTTATATACGGAAAGTGATGTTCACCCCAGTTGCAAAGTAACAGCAAT GAAGTGCTTTCTCTTGGAGTTACAAGTTATTTCACTTGAGTCCGGAGATGCAAGTAT TCATGATACAGTAGAAAATCTGATCATCCTAGCAAACAACAGTTTGTCTTCTAATGG GAATGTAACAGAATCTGGATGCAAAGAATGTGAGGAACTGGAGGAAAAAAATATTA AAGAATTTTTGCAGAGTTTTGTACATATTGTCCAAATGTTCATCAACACTTCTTGA).

In some embodiments, the isolated nucleic acid is a linear nucleic acid. In some embodiments, the isolated nucleic acid is a circular nucleic acid. In some embodiments, the isolated nucleic acid is a vector, such as a plasmid vector, an adenoviral vector, an adeno-associated viral vector, a viral vector, a retroviral vector, or a lentiviral vector. In some embodiments, the isolated nucleic acid, or an, e.g., contiguous, portion thereof containing the binding domain transmembrane domain and one or more signaling and/or costimulation endodomains is integrated into the genome of a host cell, such as a host γδ T cell. In an exemplary embodiment, the isolated nucleic acid is retroviral vector.

### γδ T Cells

Aspects of the invention include γδ T cells that functionally express an isolated nucleic acid described herein, and thereby expresses a CAR on the surface of the γδ T cell.

Aspects of the invention can additionally or alternatively include γδ T cells having in vitro or in vivo cytotoxic activity against a hematological tumor cell that exhibits cell surface expression of a desired tumor associated antigen (TAA), e.g. CD20. In some cases, the cytotoxic activity is innate activity. In some cases, the cytotoxicity is at least in part, significantly (>about 25%), or entirely, due to the presence of a CAR construct having a binding domain that specifically binds the TAA expressed on the surface of the hematological tumor cell. In some cases, the γδ T cells exhibit hematological tumor cell killing activity greater than an innate level of in vitro and/or in vivo hematological tumor cell killing activity in a control γδ T cell. In some cases, the control γδ T cell does not comprise a CAR construct. In some cases, the control γδ T cell comprises a CAR construct lacking a binding domain described herein, a hinge region described herein, a transmembrane domain described herein, a signaling domain described herein, and/or a costimulation endodomain described herein.

In some cases, the cytotoxicity is at least in part, significantly (>about 25%), or entirely, due to the presence of a CAR construct having a binding domain that specifically binds CD20 or an epitope within CD20. In some cases, the γδ T cells functionally express a CD20 specific CAR encoded by an isolated nucleic acid described herein.

In some embodiments, γδ T cells described herein can exhibit HLA-restricted (e.g., HLA class I restricted) cytotoxicity. In other embodiments, most (>50%), substantially all (>90%), or all of the cytotoxic activity is not HLA-restricted (e.g., HLA class I restricted). HLA-restricted cytotoxic activity can be assessed by comparing in vitro cytotoxicity against an HLA (e.g., HLA class I) (null) tumor cell line versus in vitro cytotoxicity against an HLA+ (e.g., HLA class I*) tumor cell line. In some embodiments, the HLA-restricted cytotoxic activity is at least in part, significantly (>25%), or entirely, provided by the use of a T cell Receptor-like binding domain. T cell receptor like binding domains are binding domains that specifically recognize the antigen when presented on the surface of a cell in complex with an MHC molecule. T cell Receptor-like binding domains are further described, e.g., in WO 2016/199141.

γδ T cells described herein can exhibit robust and/or persistent hematological tumor cell killing activity. In some cases, the hematological tumor cell killing activity can persist for at least about 6 days to 120 days, or for at least about 6 days to 180 days, from first contact with a hematological tumor cell. In some cases, the hematological tumor cell killing activity of a γδ T cell described herein, or a progeny thereof, can persist for at least about 6 days to 120 days, or for at least about 6 days to 180 days, from first contact with a hematological tumor cell, or from administration of the γδ T cell described herein. This persistent hematological tumor cell killing activity can be exhibited in vitro, in vivo, or both in vitro and in vivo.

Aspects of the invention can additionally or alternatively include γδ T cells that proliferate in response to contact with cells that exhibit cell surface expression, or overexpression, of the tumor associated antigen (TAA) (e.g., CD20). The cells that exhibit cell surface expression of the tumor associated antigen (TAA) can be normal hematological cells, such as normal B cells. The cells that exhibit cell surface expression, or overexpression, of the tumor associated antigen (TAA) can be hematological tumor cells. In some cases, the proliferation is an innate activity. In some cases, the proliferation is at least in part, significantly (>about 20% or >about 25%), or entirely, due to the presence of a CAR construct having a binding domain that specifically binds the TAA expressed on the surface of the hematological cell or hematological tumor cell. In some cases, the γδ T cells exhibit a greater level of in vitro and/or in vivo proliferation as compared to a control γδ T cell. In some cases, the control γδ T cell does not comprise a CAR construct. In some cases, the control γδ T cell comprises a CAR construct lacking a binding domain described herein, a hinge region described herein, a transmembrane domain described herein, a signaling domain described herein, and/or a costimulation endodomain described herein.

In some cases, the proliferation is at least in part, significantly (>about 20 or >about 25%), or entirely, due to the presence of a CAR construct having a binding domain that specifically binds CD20 or an epitope within CD20. In some cases, γδ T cells exhibiting proliferation in response to contact with a hematological cell or hematological tumor cell that exhibits cell surface expression of CD20 functionally express a CD20 specific CAR encoded by an isolated nucleic acid described herein.

γδ T cells described herein can exhibit robust and/or persistent proliferation in a host organism that comprises the hematological cell or hematological tumor cell that exhibits cell surface expression, or overexpression, of the tumor associated antigen (TAA) (e.g., CD20). In some cases, the proliferation can persist for at least about 6 days to 120 days, or for at least about 6 days to 180 days, from first contact with a hematological tumor cell or from a date of administration of the γδ T cell to the host organism. In some cases, the proliferation of a γδ T cell described herein, or a progeny thereof, in the host organism that comprises the hematological cell or hematological tumor cell that exhibits cell surface expression, or overexpression, of the tumor associated antigen (TAA) can persist for at least about 6 days to 120 days, or for at least about 6 days to 180 days, from first contact with a hematological cell or hematological tumor cell or from the date of first administration of the γδ T cell to the host organism. In some cases, the proliferation in the host organism is at least in part, significantly (>about 20% or >about 25%), or entirely, due to the presence of a CAR construct having a binding domain that specifically binds CD20 or an epitope within CD20. In some cases, γδ T cells exhibiting proliferation in the host organism comprising a hematological cell or hematological tumor cell that exhibits cell surface expression of CD20 functionally express a CD20 specific CAR encoded by an isolated nucleic acid described herein.

In some embodiments, the γδ T cells described herein express, or persistently express, pro-inflammatory cytokines such as, but not limited to, tumor necrosis factor alpha or interferon gamma after contact with the hematological cell or hematological tumor cell. In some embodiments, the γδ T cells described herein, or progeny thereof, express, or persistently express, pro-inflammatory cytokines such as tumor necrosis factor alpha or interferon gamma after contact with the hematological cell or hematological tumor cell, e.g., in a host organism comprising the hematological cell or hematological tumor cell.

In some embodiments, the γδ T cell, or a pharmaceutical composition containing the γδ T cell, exhibits essentially no, or no graft versus host response when introduced into an allogeneic host. In some embodiments, the γδ T cell, or a pharmaceutical composition containing the γδ T cell, exhibits a clinically acceptable level of graft versus host response when introduced into an allogeneic host. In some embodiments, a clinically acceptable level is an amount of graft versus host response that does not require cessation of a γδ T cell treatment to achieve a therapeutically effective treatment. In some embodiments, a clinically acceptable level of graft versus host response (GvHD) is an acute response that is less severe than Grade C according to an applicable IBMTR grading scale. The severity of acute graft versus host response is determined by an assessment of the degree of involvement of the skin, liver, and gastrointestinal tract. The stages of individual organ involvement are combined to produce an overall grade, which has prognostic significance. Grade I (A) GvHD is characterized as mild disease, grade II (B) GvHD as moderate, grade III (C) as severe, and grade IV (D) life-threatening. The IBMTR grading system defines the severity of acute GvHD as follows (Rowlings et al., Br J Haematol 1997; 97:855):
Grade A—Stage 1 skin involvement alone (maculopapular rash over <25 percent of the body) with no liver or gastrointestinal involvement
Grade B—Stage 2 skin involvement; Stage 1 to 2 gut or liver involvement
Grade C—Stage 3 involvement of any organ system (generalized erythroderma; bilirubin 6.1 to 15.0 mg/dL; diarrhea 1500 to 2000 mL/day)
Grade D—Stage 4 involvement of any organ system (generalized erythroderma with bullous formation; bilirubin >15 mg/dL; diarrhea>2000 mL/day OR pain OR ileus). See also, Schoemans et al., Bone Marrow Transplantation volume 53, pages1401-1415 (2018), e.g., at Tables 1 and 2, which discloses criteria for assessing and grading acute GvHD.

In some embodiments, the γδ T cell, or a pharmaceutical composition containing the γδ T cell, exhibits reduced or substantially reduced graft versus host response when introduced into an allogeneic host as compared to a graft versus host response exhibited by control αβ T cells, or a control pharmaceutical composition comprising the control αβ T cells, administered to an allogeneic host. In some cases, the control αβ T cell is an allogeneic non-engineered control αβ T cell. In some cases, the control αβ T cell does not comprise a CAR or does not comprise the same CAR as a reference γδ T cell.

The γδ T cells described herein can be δ1, δ2, δ3, or δ4 γδ T cells, or combinations thereof. In some cases, the γδ T cells are mostly (>50%), substantially (>90%), essentially all, or entirely δ2⁻γδ T cells. In some cases, the γδ T cells are mostly (>50%), substantially (>90%), essentially all, or entirely δ1 γδ T cells.

γδ T cells can be obtained from an allogeneic or an autologous donor. The γδ T cells can be, partially or entirely purified, or not purified, and expanded ex vivo. Methods and compositions for ex vivo expansion include, without limitation, those described in WO 2017/197347. The expansion may be performed before or after, or before and after, a CAR construct is introduced into the γδ T cell(s).

γδ T cells described herein can be stored, e.g., cryopreserved, for use in adoptive cell transfer.

### Biomarkers, Methods of Detection, and Use Thereof

Biomarkers are biological indicators of disease or therapeutic effects that can be measured in vivo by biomedical/molecular imaging, as well as other in vitro or laboratory methodologies. As disclosed herein, one or more biomarkers can advantageously be relied upon to inform cell activation, treatment efficacy and/or follow-on treatment regimens. With respect to administration of anti-CD20 CAR γδ T cells to a subject in need thereof as herein described, one or more biomarkers can be relied upon as indicator(s) of effectiveness, potential for effectiveness, or lack thereof in terms of, e.g., promoting an anti-tumor effect in the subject. In embodiments, one or more biomarkers can be relied upon to determine, for example, whether to administer one or more additional dosing regimens, and if so, whether to adjust a dosage level (e.g., increase, decrease, or maintain the same dosage of anti-CD20 CAR γδ T cells), to include one or more additional or alternative therapies, to adjust a previously planned dosing schedule, to administer anti-CD20 CAR γδ T cells derived from a same or a different donor, or whether to halt/postpone treatment or discontinue treatment altogether, and the like.

In embodiments, activation and/or expansion of an administered anti-CD20 CAR γδ T cells can be monitored by way of flow cytometry detection of CAR+γδ T cells and/or via quantitative polymerase chain reaction (qPCR) detection of the anti-CD20 CAR transgene. Preferably, such methodology(s) are conducted at a number of time points following administration of the anti-CD20 CAR γδ T cells, for example daily, every other day, every 3 days, every 4 days, etc., up to e.g., 14 days, 28 days, 2 months, 3 months, or more, as it is well known that the presence and the status of CAR-T cells in peripheral blood can vary over time (Shah et al. (2020) Nat Med., 26:1569-75). In the art, PCR results have been reported to correlate with CAR surface expression as monitored by flow cytometry, however flow cytometry has advantages in that it allows for identification and characterization of CAR-T cell subpopulations and of a patient's immune cells in a fast way and at a single cell level. In addition, flow cytometry detects the CAR at the proteomic level and thus can provide information regarding CAR cell functionality. The experimental manner in how to rely on one or more of flow cytometry, qPCR and/or other detection methodologies to monitor activation and/or expansion of anti-CD20 CAR γδ T cells is readily determined by the skilled artisan, as shown and described, e.g., in Example 1 below, and as described e.g., by Hu and Huang (2020) Front. Immunol., 11(1770).

In embodiments, anti-CD20 CAR γδ T cells administered to a subject can induce release of one or more cytokines. In embodiments, the one or more cytokines are secreted from the anti-CD20 CAR γδ T cells. In additional or alternative embodiments, the one or more cytokines are secreted from cells other than the anti-CD20 CAR γδ T cells including, e.g., T cells, NK cells, dendritic cells, and macrophages. In embodiments, induction of one or more inflammatory cytokines mitigates immunosuppression caused by a tumor microenvironment, and can in turn improve clinical response to the anti-CD20 CAR γδ T cell therapy.

In embodiments, one or more cytokines are biomarkers of cell activation and/or therapeutic efficacy of an anti-CD20 CAR γδ T cell therapy as herein disclosed. Relevant cytokines can include but are not limited to INFγ, GM-CSF, IL-2, IL-7, IL-15, TNFa, IL-1B, IL-6, IL-8, IL-10, MIP1α, MIP1β, CRP, ferritin, monocyte chemotactic protein-1 (MCP-1), CXCL9, CXCL10, CXCL11, CCL5, IL-5, IL-IRA, IL-18, soluble MICA, IL-10, IL-4, IL-13, IL-17, CCL2, CXCL12, CCL17, and CCL22. In preferred embodiments, the one or more cytokines comprise or consist of IL-2 and IL-8.

In embodiments, induction of cytokines for use as biomarkers of therapeutic efficacy occurs within a timeframe between one day or less and 28 days following administration of anti-CD20 CAR γδ T cells. In embodiments, said timeframe is between one day or less and 21 days, or 18 days, or 14 days, or 10 days following administration of anti-CD20 CAR γδ T cells. In some embodiments, a cytokine biomarker comprises IL-8 and induction of IL-8 occurs between one day or less and 28 days, for example between one day or less and 21 days, for example between one day or less and 14 days following administration of anti-CD20 CAR γδ T cells. In some additional or alternative embodiments, a cytokine biomarker comprises IL-2 and induction of IL-2 occurs between one day or less and 28 days, for example between one day or less and 21 days, for example between one day or less and 14 days following administration of anti-CD20 CAR γδ T cells. In some additional or alternative embodiments, induction of cytokines for use as biomarkers occurs within a timeframe between one day or less following LD and 28 days following administration of anti-CD20 CAR γδ T cells. In embodiments, such a timeframe is between one day or less following LD and 21 days, or 18 days, or 14 days, or 10 days following administration of anti-CD20 CAR γδ T cells. In some embodiments, a cytokine biomarker comprises IL-8 and induction of IL-8 occurs between one day or less following LD and 28 days following administration of anti-CD20 CAR γδ T cells, for example between one day or less following LD and 21 days following administration of anti-CD20 CAR γδ T cells, for example between one day or less following LD and 14 days following administration of anti-CD20 CAR γδ T cells. In some additional or alternative embodiments, a cytokine biomarker comprises IL-2 and induction of IL-2 occurs between one day or less following LD and 28 days following administration of anti-CD20 CAR γδ T cells, for example between one day or less following LD and21 days following administration of anti-CD20 CAR γδ T cells, for example between one day or less following LD and 14 days following administration of anti-CD20 CAR γδ T cells.

Measurement of serum levels of single cytokines are commonly performed using enzyme-linked immunosorbent assay (ELISA) and/or chemiluminesent assays, and multiplex bead-based assays can be used to determine serum levels of a plurality of cytokines in a single test (Knight et al. (2020) Archives of Pathology & Laboratory Medicine, 144(10)). In embodiments, serum levels of one or more cytokines are measured before administration of anti-CD20 CAR γδ T cells, e.g., before lymphodepletion and/or following/during lymphodepletion but prior to anti-CD20 CAR γδ T cells. In additional or alternative embodiments, serum levels of one or more cytokines are measured following administration of anti-CD20 CAR γδ T cells. In embodiments, serum levels of one or more cytokines for use as biomarkers are measured before administration of anti-CD20 CAR γδ T cells (e.g., between 1 and 7 days prior to administration), and/or are measured one or more times following administration of anti-CD20 CAR γδ T cells up to about 28 days. In embodiments, a plurality of measurements of serum levels of one or more cytokines encompassing a timeframe before and/or following administration of anti-CD20 CAR γδ T cells provides a time course of induction of the one or more cytokines. Such a time course can be used to establish peak serum levels of said one or more cytokines and/or the time course can be used to establish approximate total levels of cytokine induction during the time course. It is within the scope of this disclosure that peak levels of one or more cytokines are used as a biomarker metric. Additionally or alternatively, it is within the scope of this disclosure that total levels of release of one or more cytokines are used as a biomarker metric.

In embodiments, a presence of a biomarker (e.g., a cytokine) is confirmed in response to said biomarker being measured above some predetermined threshold, for example following administration of anti-CD20 CAR γδ T cells. In embodiments, a biomarker is IL-8, and the presence of the biomarker is confirmed responsive to serum levels of IL-8 reaching or exceeding about 100 pg/mL, or about 125 pg/mL, or about 150 pg/mL, or about 175 pg/mL, or about 200 pg/mL within a predetermined timeframe (e.g., 21 days or less) following administration of anti-CD20 CAR γδ T cells. In additional or alternative embodiments, a biomarker is IL-2 and the presence of the biomarker is confirmed responsive to serum levels of IL-2 reaching or exceeding about 75 pg/mL, or about 80 pg/mL, or about 85 pg/mL within a predetermined timeframe (e.g., 21 days or less) following administration of anti-CD20 CAR γδ T cells.

In embodiments, confirmation of the presence of one or more cytokine biomarkers as herein described is used to inform follow-on treatments. For example, in response to cytokine biomarker confirmation following administration of a first dose of anti-CD20 CAR γδ T cells, a second dose may be optional, or a dosage of the corresponding second dose may be adjusted accordingly (e.g., maintained the same as the first dose or decreased). In additional or alternative embodiments, a lack of cytokine biomarker confirmation following administration of a first dose of anti-CD20 CAR γδ T cells may indicate a need for a second dose (e.g., with or without another lymphodepletion step), that a cell dosage amount be increased for said second dose, and/or that the second dose comprise anti-CD20 CAR γδ T cells derived from a different donor as compared to the first dose. Similar logic additionally or alternatively applies to an indication of presence or absence of biomarkers indicative of in vivo activation and/or expansion of administered anti-CD20 CAR γδ T cells as described above.

In additional or alternative embodiments, indicators of minimal residual disease (MRD) can serve as a biomarker for informing follow-on treatment regimens. Discussed herein, MRD refers to some amount of cancer cells remaining in the body of a subject following a course of treatment (e.g., administration of one or more doses of anti-CD20 CAR γδ T cells). In embodiments, MRD analysis is conducted some predetermined time duration following a last administration of anti-CD20 CAR γδ T cells. In embodiments, said time duration is at least 20 days, for example at least 25 days, for example at least 28 days, for example at least 30 days following a last administration of anti-CD20 CAR γδ T cells. In embodiments, an MRD positive test is indicative of disease continuing to be detected following treatment, whereas an MRD negative test is indicative of disease not being detected following treatment. In embodiments, an MRD positive test can indicate a need for an additional treatment regimen, for example a second course of treatment comprising administration of another round of anti-CD20 CAR γδ T cells, preferably at a higher cell dosage, preferably including an additional lymphodepletion step. In some embodiments, a first course of treatment comprising administration of anti-CD20 CAR γδ T cells may follow a standard course of lymphodepletion (e.g., comprising or consisting of fludarabine at 30 mg/m²/day plus cyclosporamide at 500 mg/m²/day for three days), and responsive to an MRD positive test, a second course of treatment may comprise an enhanced lymphodepletion step (e.g., comprising or consisting of fludarabine at 30 mg/m²/day for four days plus cyclosporamide at 1000 mg/m²/day for three days).

In embodiments, MRD analysis is conducted via one or more of multiparametric flow cytometry and immunosequencing as known in the art (see, e.g., Wood et al. (2018) Blood, 131(12): 1350-1359).

### Methods of Inhibiting or Killing Tumor Cells

One or multiple non-engineered, γδ T-cell populations, engineered, γδ T-cell populations, and/or admixtures thereof, having cytotoxic activity against a hematological tumor cell can be administered to a subject in any order or simultaneously. If simultaneously, the multiple non-engineered, γδ T-cell population, engineered, γδ T-cell population, and/or admixtures thereof, of the invention can be provided in a single, unified form, such as an intravenous injection, or in multiple forms, for example, as multiple intravenous infusions, s.c, injections or pills. The non-engineered, γδ T-cell population, engineered, γδ T-cell population, and/or admixtures thereof, of the invention can be packed together or separately, in a single package or in a plurality of packages. One or all of the non-engineered γδ T-cell population, engineered γδ T-cell population, and/or admixtures thereof, of the invention can be given in multiple doses. If not simultaneous, the timing between the multiple doses may vary to as much as about a week, a month, two months, three months, four months, five months, six months, or about a year. In some cases, a non-engineered, enriched γδ T-cell population, an engineered, enriched γδ T-cell population, and/or admixtures thereof, of the invention can proliferate within a subject's body, in vivo, after administration to a subject. One or more non-engineered γδ T-cell populations, one or more engineered γδ T-cell populations, and/or admixtures thereof, can be frozen to provide cells for multiple treatments with the same cell preparation. One or more non-engineered γδ T-cell populations, one or more engineered γδ T-cell populations, and/or admixtures thereof, of the disclosure, and pharmaceutical compositions comprising the same, can be packaged as a kit. A kit may include instructions (e.g., written instructions) on the use of the non-engineered γδ T-cell population, the engineered γδ T-cell population, and/or admixtures thereof, and compositions comprising the same.

In some cases, a method of treating a B cell malignancy comprises administering to a subject a therapeutically-effective amount of a non-engineered γδ T-cell population, an engineered γδ T-cell population, and/or admixtures thereof, wherein the administration treats the B cell malignancy (e.g., expressing CD20 on the cell surface). In some embodiments the therapeutically-effective amount of the non-engineered, γδ T-cell population, the engineered γδ T-cell population, and/or admixtures thereof, is administered for at least about 10 seconds, 30 seconds, 1 minute, 10 minutes, 30 minutes, 1 hour, 2 hours, 3 hours, 4 hours, 5 hours, 8 hours, 12 hours, 24 hours, 2 days, 3 days, 4 days, 5 days, 8 days, 1 week, 2 weeks, 3 weeks, 1 month, 2 months, 3 months, 4 months, 5 months, 6 months, or 1 year. In some embodiments the therapeutically-effective amount of the non-engineered γδ T-cell population, the engineered γδ T-cell population, and/or admixtures thereof, is administered for at least one week. In some embodiments the therapeutically-effective amount of the non-engineered γδ T-cell population, the engineered γδ T-cell population, and/or admixtures thereof, is administered for at least two weeks.

A non-engineered γδ T-cell population, an engineered γδ T-cell population, and/or admixtures thereof, described herein can be administered before, during, or after the occurrence of a disease or condition, and the timing of administering a pharmaceutical composition containing the γδ T-cell population can vary. For example, the γδ T-cell population can be used as a prophylactic and can be administered continuously to subjects with a propensity to conditions or diseases in order to lessen a likelihood of the occurrence of the disease or condition. The initial administration can be via any route practical, such as by any route described herein using any formulation described herein. In some examples, the administration of a γδ T-cell population of the disclosure is an intravenous administration. One or multiple dosages of the γδ T-cell population can be administered as soon as is practicable after the onset of a hematological cancer and for a length of time necessary for the treatment of the immune disease, such as, for example, from about 24 hours to about 48 hours, from about 48 hours to about 1 week, from about 1 week to about 2 weeks, from about 2 weeks to about 1 month, from about 1 month to about 3 months. In some embodiments, one or multiple dosages of the γδ T-cell population can be administered years after onset of the cancer and before or after other treatments.

In some embodiments, the γδ T-cell population is administered simultaneously or sequentially with one or more methods to elevate common gamma chain cytokine(s). As used herein, “one or more methods to elevate common gamma chain cytokine(s): refers to a method, or combination of methods, that alters the physiological state of a subject, such that at least one common gamma chain cytokine level is elevated in the subject. In some embodiments, the method elevates the level of one or more common gamma chain cytokine(s) selected from the group consisting of IL-2, IL-7, and IL-15, preferably wherein the method elevates the level of IL-15 in the subject. In some embodiments, the method comprises lymphodepletion. In some embodiments, the method comprises administering one or more common gamma chain cytokine(s) to the subject. In some cases, IL-2, IL-7, and/or IL-15, preferably IL-15, are administered. In some embodiments, the method comprises secreting common gamma chain cytokine(s) from an administered, e.g., γδ T cell. In some cases, IL-2, IL-7, and/or IL-15, preferably IL-15, are secreted.

In some embodiments, the administering one or more methods to elevate common gamma chain cytokine(s) comprises lymphodepletion before introducing the γδ T cell(s). In some embodiments, the administering one or more methods to elevate common gamma chain cytokine(s) comprises administering simultaneously with introducing the γδ T cell(s) or sequentially an amount of common gamma chain cytokine(s) effective to increase proliferation, cytotoxic activity, persistence, or the combination thereof of the introduced γδ T cell(s), preferably wherein the method comprises administering IL-2 or one or more mimetics thereof, more preferably wherein the method comprises administering IL-15 or one or more mimetics thereof. The amount of administered common gamma chain cytokine(s) can be an amount effective to increase proliferation, cytotoxic activity, persistence, or the combination thereof of the introduced γδ T cell(s) before and/or after introducing the γδ T cell(s). Exemplary amounts of IL-15 include, without limitation between 0.01-10 ug/kg/dose every 24 hours for IL-15. Exemplary amounts of IL-2 include, without limitation, between about 3x106 and about 22×106 units every 8-48 hours. For example, the dosing regimen for IL2 in RCC is 600,000 International Units/kg (0.037 mg/kg) IV q8hr infused over 15 minutes for a maximum 14 doses.

In some embodiments, the administering one or more methods to elevate common gamma chain cytokine(s) comprises lymphodepletion before administering the γδ T cell(s) and administering simultaneously with introducing the γδ T cell(s) or sequentially an amount of common gamma chain cytokine(s) effective to increase proliferation, cytotoxic activity, persistence, or the combination thereof of the introduced γδ T cell(s).

It should be understood that other suitable lymphodepletion methods can be utilized in the methods of the present disclosure. Exemplary lymphodepletion methods are disclosed in, for example, Amini, et al., “Preparing for CAR T cell therapy: patient selection, bridging therapies and lymphodepletion,” Nat. Rev. Clin. Oncol. 19(5): 342-355 (May 2022) and Bechman, N. and Maher, J., “Lymphodepletion strategies to potentiate adoptive T-cell immunotherapy—what are we doing; where are we going,” Expert Opin. Biol. Ther. 21(5): 627-637 (May 2021), each of which is incorporated herein by reference in its entirety.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Phase I anti-CD20 γδ CAR T Cells in Humans

This Example demonstrates safety profile, evidence of cell expansion, and pharmacodynamics engagement in regards to anti-CD20 allogeneic γδ CAR T cell therapy.

A first-in-human (FIH) study was conducted on a set of NHL patients relapsing from at least three prior lines of treatment. Six patients were enrolled. Five of the patients were dosed at 3×10⁷ CAR+ cells, but two of the five patients enrolled were non-dose limiting toxicity evaluable and hence were excluded from further analysis. Of the six patients initially enrolled, one patient was dosed at 1×10⁸ CAR+ cells. Thus, in the context of the study, a safety subset included 6 patients (all patients that received the anti-CD20 allogeneic γδ CAR T cell therapy), and an efficacy subset included 4 patients that completed at least one response assessment.

The study design was comprised of four stages. The first stage (day −5 to day 0) included a step of lymphodepletion (LD), followed by treatment in the second stage (day 0 to day 28). The second stage comprised a dose escalation study with the anti-CD20 allogeneic γδ CAR T cell therapy. At the end of the second stage, patient response and a safety assessment was conducted. The third stage (day 28 to month 12) included an initial follow-up stage (response and safety assessment at months 3, 6, 9, and 12). The fourth stage included a long-term follow-up study. LD in the first stage comprised either standard LD (sLD), or enhanced LD (eLD). sLD included administration of fludarabine at 30 mg/m²/day plus cyclosporamide at 500 mg/m²/day for three days. eLD included fludarabine at 30 mg/m²/day for four days plus cyclosporamide at 1000 mg/m²/day for three days.

### Patient Characteristics

Subject 101-104-001 is a female, age 62 with transformed DLBCL (from CLL). This subject was previously treated with five prior lines of therapy with progressive disease (PD) as the best response, the five prior lines of therapy including 1) R-CHOP, 2) rituximab-abbs, gemcitabine, and CDDP, 3) rituximab-abbs, gemcitabine, carboplatin, 4) polatuzumab+BR×2, and 5) obinutuzumab-hyper cyclophosphamide and dexamethasone. For the study, this subject received sLD and was administered 3×10⁷ anti-CD20 allogeneic γδ CAR T cells.

Subject 101-108-009 is a female, age 66 with transformed high grade B cell tumor (from FL). This subject was previously treated with four prior lines of therapy that included 1) R-CHOP, 2) ibrutinib, 3) bendamustine/rituximab, and 4) rituximab. For the study, this subject received sLD and was administered 3×10⁷ anti-CD20 allogeneic γδ CAR T cells.

Subject 101-108-010 is a male, age 75 with DLBCL. This subject was previously treated with five prior lines of therapy that included 1) R-CHOP; IT MTX, 2) liso-cel, 3) liso-cel (reinfusion), 4) revlimid, and 5) tafasitamab-cxix. For the study, this subject received eLD and was administered 3×10⁷ anti-CD20 allogeneic γδ CAR T cells.

Subject 101-108-012 is a male, age 62 with MCL. This subject was previously treated with five prior lines of therapy that included 1) bendamustine/rituximab, 2) zanubrutinib, 3) bendamustine/obinutuzumab, 4) bendamustine/rituximab, and 5) rituximab/gemcitabine/dex/carboplatin. For the study, this subject received eLD and was administered 1×10⁸ anti-CD20 allogeneic γδ CAR T cells.

All of the above subjects completed the DLT protocol. The two patients below did not complete the DLT period, but are included herein for reference.

Subject 101-104-002 is a male, age 29 with Primary Refractory Burkitt Lymphoma. This subject was previously treated with three prior lines of therapy that included 1) R-CODOC-M/R-IVAC, 2) cy/flu/rituximab+NK cell trial (FK516), and 3) R-EPOCH. For the study, this subject received sLD and was administered 3×10⁷ anti-CD20 allogeneic γδ CAR T cells.

Subject 101-102-004 is a male, age 52 with Double hit DLBCL. This subject was previously treated with five prior lines of therapy that included 1) DA-EPOCH R with IT MTX/ARA-C, 2) R-ICE, 3) polatuzumab/rituximab, 4) tisagenlecleucel, and 5) gemcitabine/oxliplatin. For the study, this subject received sLD and was administered 3×10⁷ anti-CD20 allogeneic γδ CAR T cells.

Table 1 below illustrates the safety profile data corresponding to the study.

TABLE 1
FIH Study Safety Profile

| Treatment-emergent | | |
|---|---|---|
| adverse events (TEAE) | All grades (%) | Grade 3+ (%) |
| Infusion-related reaction | (0%) | (0%) |
| (IRR) | | |
| Cytokine release syndrome | 3 (50%) | (0%) |
| (CRS) | | |
| Immune effector cell | (0%) | (0%) |
| associated neurotoxicity | | |
| (ICANS) | | |
| Graft vs Host Disease | (0%) | (0%) |
| (GvHD) | | |
| Infection | 2 (33%) | 1 (17%) |

With regard to the data depicted at Table 1, the data corresponds to an N of 6 (all enrolled patients). No DLTs were observed, no ICANs were observed, no GvHD was observed, on no grade 3+ CRS was observed. In terms of the infection sub-category, one subject had COVID-19 and pneumonia, and one subject had candida.

Table 2 below illustrates the responses obtained in efficacy-evaluable patients.

TABLE 2
FIH Study Responses in Efficacy-Evaluable Patients

| Subject | Best Response |
|---|---|
| 101-104-001 | Progressive disease (PD) |
| 101-108-009 | Partial Response (PR) (near complete response |
| | (CR)) |
| 101-108-010 | CR |
| 101-108-012 | CR |

Hence, the data depicted in Table 2 illustrates an overall response rate (ORR) of 75% (3/4 patients) and a CR of 50% (2/4 patients).

### Anti-Tumor Response

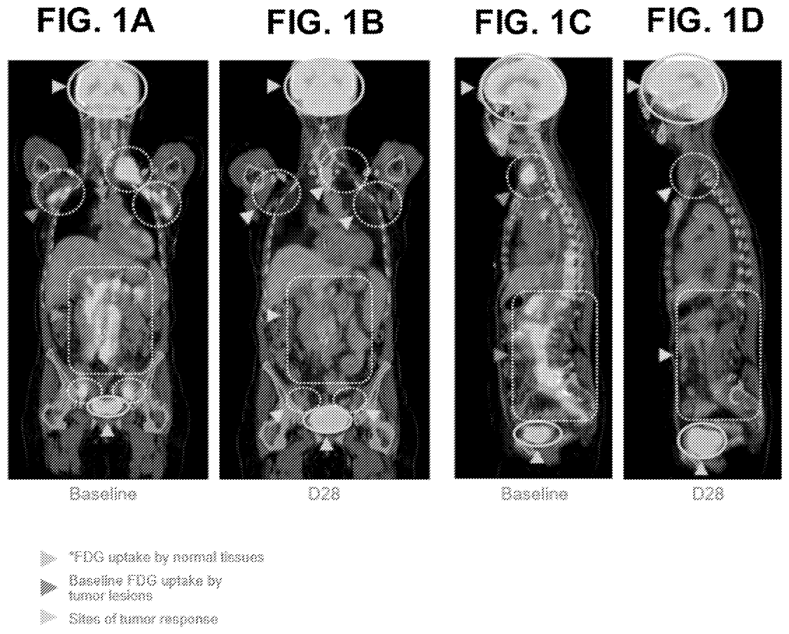

Fluorodeoxyglucose (FDG)-positron emission tomography (PET) was used to monitor cancerous lesions before and after treatment of subjects enrolled in the study with the anti-CD20 allogeneic γδ CAR T cells. FIGS. 1A-1D illustrate data obtained from subject 101-108-009. FIGS. 1A and 1C illustrate baseline FDG uptake by tumor lesions as imaged from a front and side view, respectively, and FIGS. 1B and 1D illustrate sites of tumor response as obtained on day 28 following administration of the anti-CD20 allogeneic γδ CAR T cells, from a front and side view, respectively. Each of FIGS. 1A-1D illustrate FDG uptake by normal tissues as well. Tumor response was assessed as immune-related response (PR) per Lugano 2014. This subject had a near complete response (refer to Table 2 above) from administration of 3×10⁷ anti-CD20 allogeneic γδ CAR T cells.

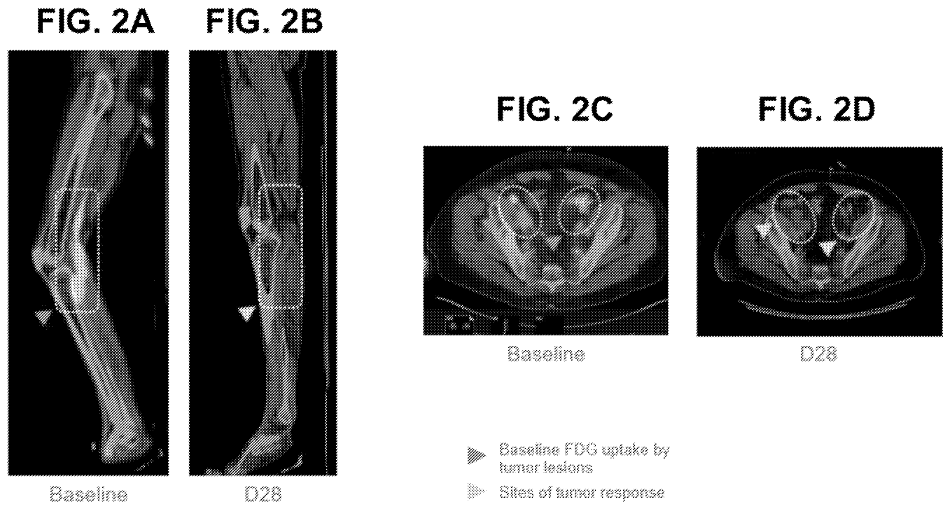

FIGS. 2A-2D illustrate FDG-PET data obtained from subject 101-108-010. FIGS. 2A and 2C illustrate baseline FDG uptake by tumor lesions as shown from a sagittal view of the right leg, and transverse view of the pelvis, respectively. FIGS. 2B and 2D illustrate sites of tumor response as obtained on day 28 following administration of the anti-CD20 allogeneic γδ CAR T cells from a sagittal view of the right leg, and transverse view of the pelvis, respectively. This subject had a complete response (refer to Table 2 above) from administration of 3×10⁷ anti-CD20 allogeneic γδ CAR T cells.

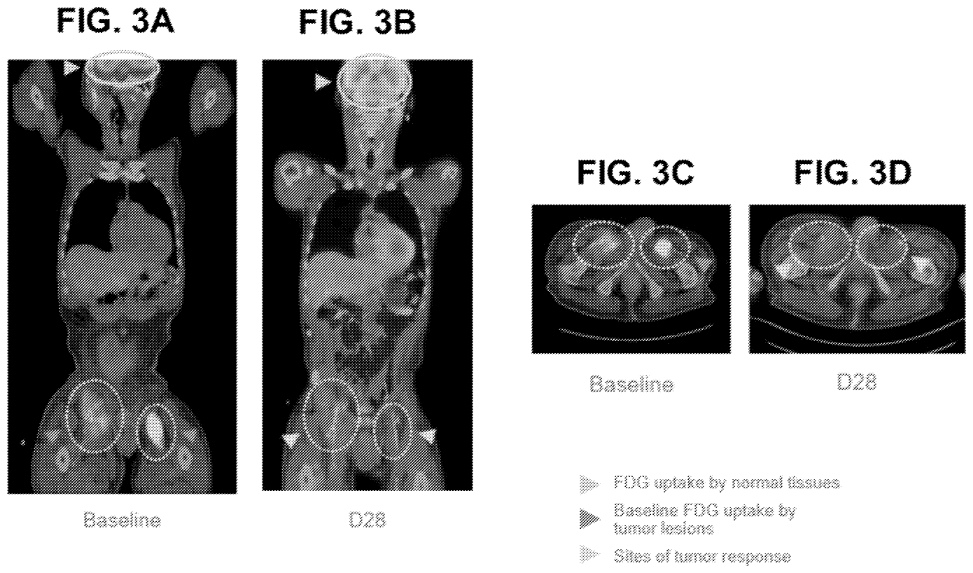

FIGS. 3A-3D illustrate FDG-PET data obtained from subject 101-108-012. FIGS. 3A and 3C illustrate baseline FDG uptake by tumor lesions as shown from a front view, and a transverse view of the pelvis, respectively. FIGS. 3B and 3D illustrate sites of tumor response as obtained on day 28 following administration of the anti-CD20 allogeneic γδ CAR T cells from a front view, and transverse view of the pelvis, respectively. FIGS. 3A-3B illustrate FDG uptake by normal tissues as well. This subject had a complete response (refer to Table 2 above) from administration of 1×10⁸ anti-CD20 allogeneic γδ CAR T cells.

### In Vivo Expansion and Pharmacodynamic Biomarkers

### Pharmacodynamic Biomarkers

Blood samples for serum cytokine analysis was collected on Day-5 prior to lymphodepletion, on Day −1 after lymphodepletion, on Day 1 prior to infusion and 2 hours and 6 hours post-infusion (+/−15 minutes), and on Days 2, 3, 5, 7, 10, 11, 1, 21, 28, and month 3 (+/−1 day) after administration of the anti-CD20 allogeneic γδ CAR T cells. For the analysis, the collected samples were shipped to a central laboratory.

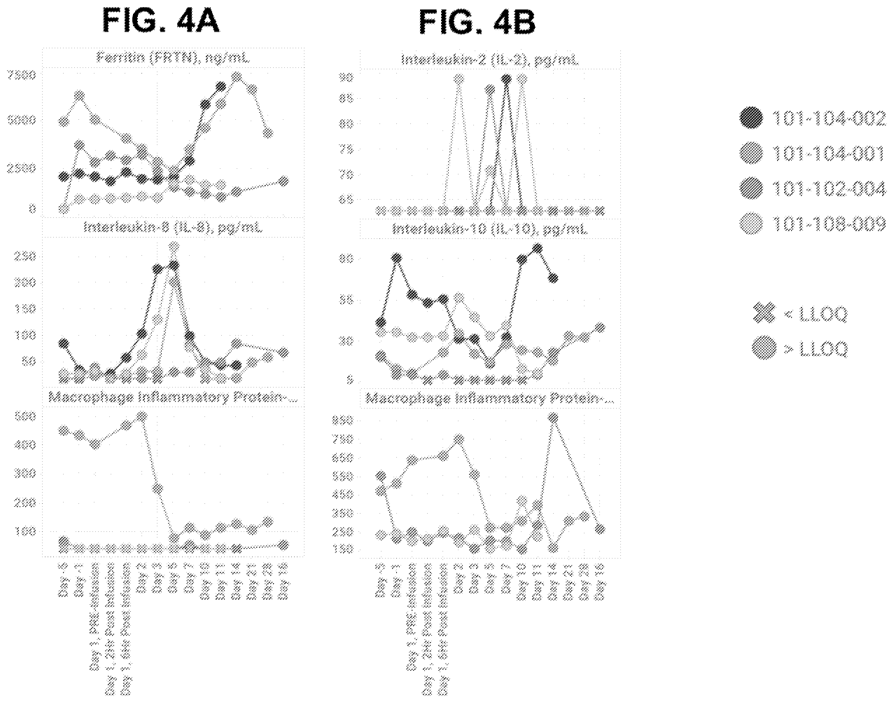

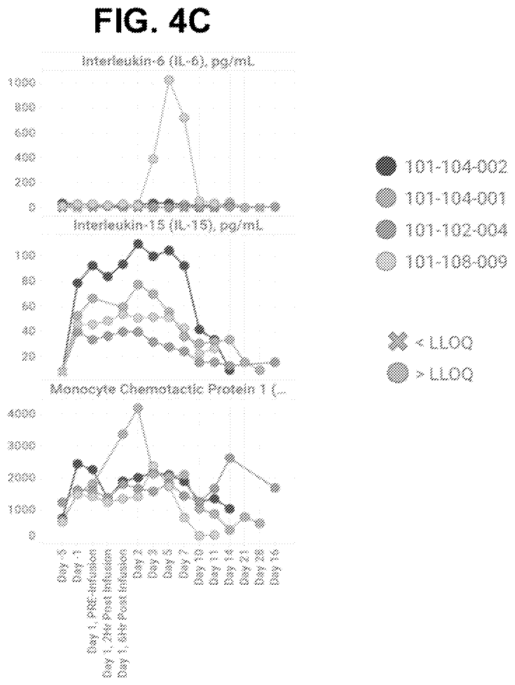

The results are shown graphically in FIGS. 4A-4C for subjects 101-104-002, 101-104-001, 101-102-004, and 101-108-009. Data points in the shape of an “X” were below a lower level of quantitation (<LLOQ).

### In Vivo Expansion

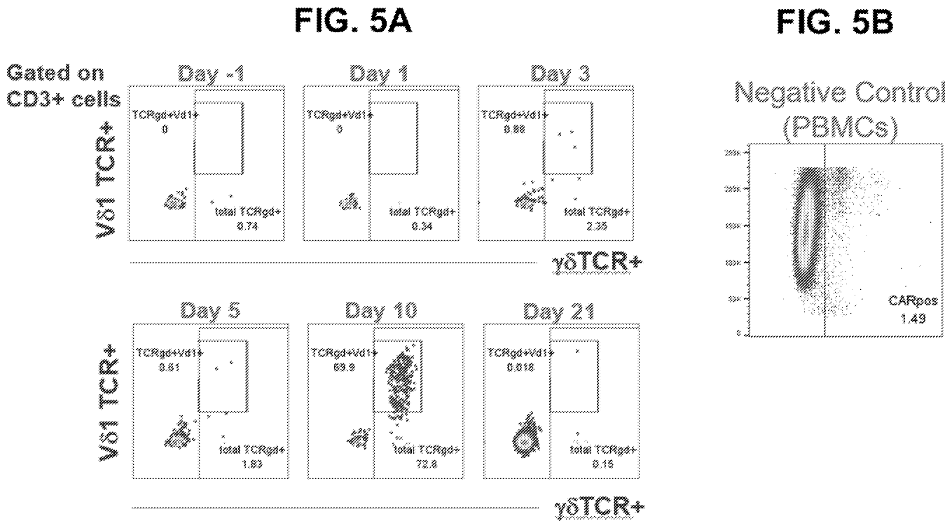

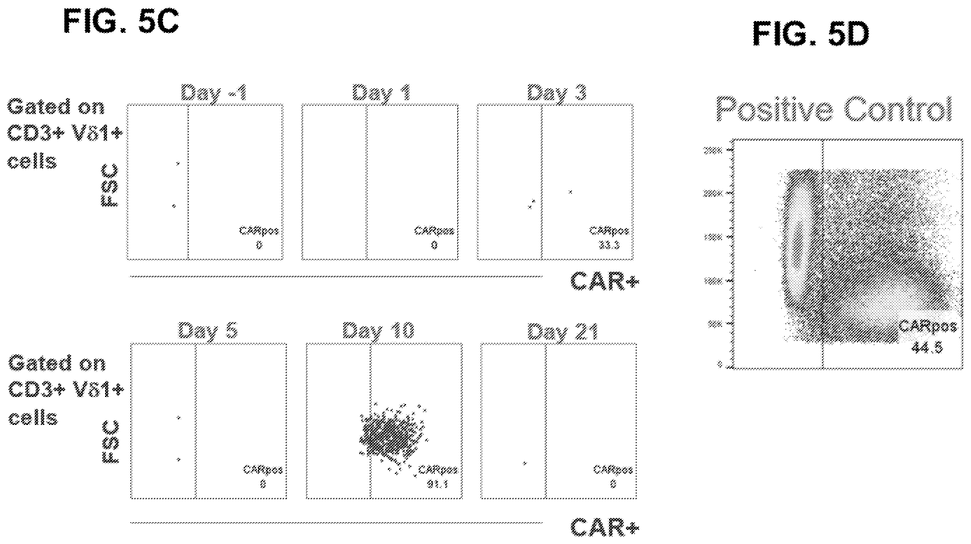

Anti-CD20 allogeneic γδ CAR T cell levels in peripheral blood were measured via flow cytometry on Day −1, Day 1, Day 3, Day 5, Day 10, and Day 21. FIG. 5A illustrates flow cytometry data gated on CD3+ cells, and FIG. 5B illustrates a negative control using PBMCs. FIG. 5C illustrates flow cytometry data gated on CD3+ and Vδ1+ cells, and FIG. 5D illustrates a positive control using CAR+Jurkat cells spiked into PBMCs (gated on CD3+ cells). The data is indicative of in vivo expansion of CAR+γδ T cells.

### Clinical Trial Update

Methods—This multicenter phase 1 clinical trial is evaluating ADI-001 in adults with relapsed/refractory B-cell lymphoma. Eligibility criteria included the presence of measurable lesions, expression of CD20 on tumor cells and ≥2 prior systemic therapies. All patients received conditioning therapy with fludarabine and cyclophosphamide. ADI-001 can be administered at four dose levels (DL) (DL1: 3E7, DL2: 1E8, DL3: 3E8 and DL4: 1E9 CAR+ cells) in a 3+3 dose-escalation scheme. Patients who completed the 28-day DLT period were considered evaluable. In DL3, patients could receive a second course of conditioning therapy and be re-dosed with ADI-001 if there was no DLT during the first 28 days, no progressive disease on PET/CT assessment on Day 28, and have recovered from cytopenias. Treatment-emergent adverse events were graded by CTCAE v5.0, and Immune Effector Cell Associated Neurologic Syndrome (ICANS) and Cytokine Release Syndrome (CRS) assessments were performed per ASTCT criteria. Objective response rates (ORR) were evaluated by independent radiographic review per Lugano 2014 criteria.

Results—As of 15 Jul. 2022, 11 patients were enrolled and nine were evaluable. Of these nine patients, six (67%) were male and the median age was 62 years (range 45-75). Eight patients had large B-cell lymphoma (LBCL) and one had mantle cell lymphoma (MCL). Of the eight patients with LBCL, five had diffuse-large B-cell lymphoma (DLBCL), two had high-grade B-cell lymphoma (HGBCL) with double/triple hit, and one had HGBCL not otherwise specified. At baseline, the median tumor burden was 2,974 (150-7,919) mm2, and 89% (8/9) had stage III/IV disease. The median number of prior therapies was four (range 2-5). Four patients had prior anti-CD19 CAR T cell therapy (two Liso-cel and two Axi-cel). Among nine evaluable patients, three patients were treated at each of DL1, DL2, and DL3. Two patients at DL3 were re-dosed with a second course of ADI-001.

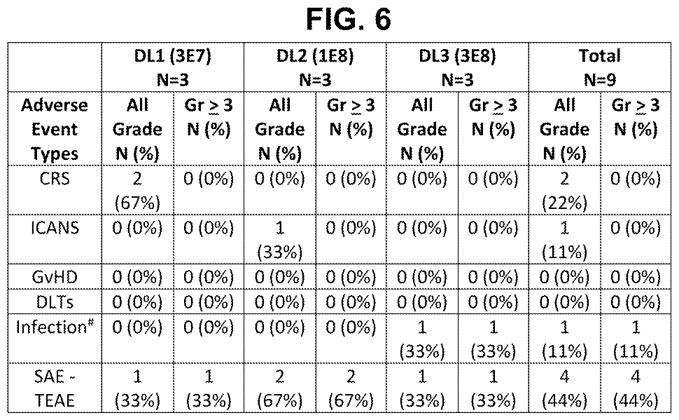

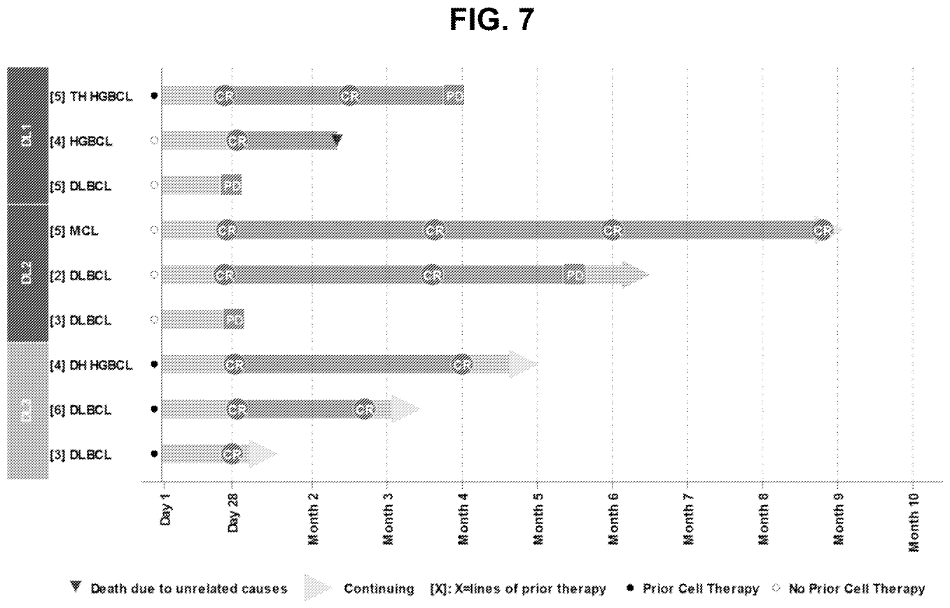

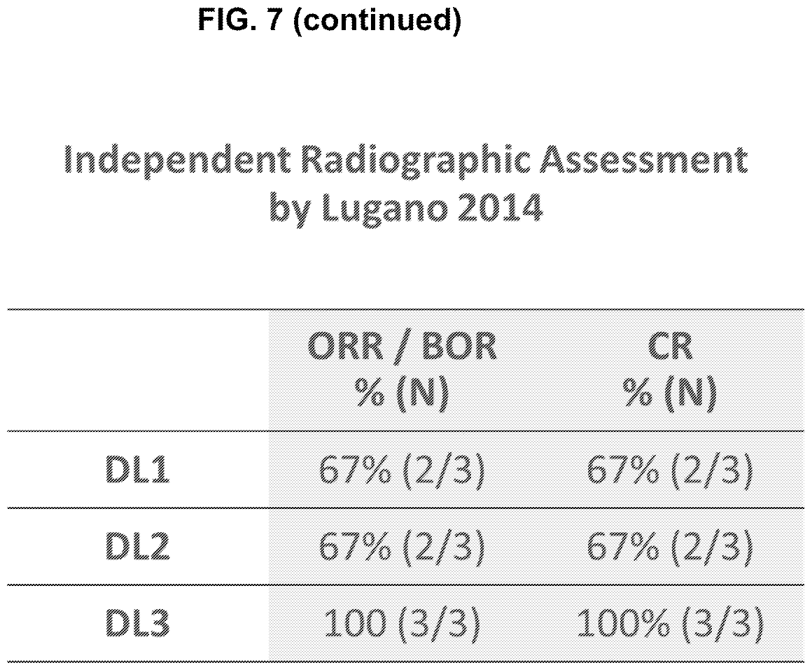

As shown in FIG. 6, two patients developed CRS: one Grade 1 and one Grade 2. One patient developed a Grade 1 ICANS which resolved within 24 hours. There were no ≥Grade 3 CRS or ICANS. The only related SAEs were Grade 2 CRS, Grade 1 ICANS and Grade 3 adenoviraemia. There was no reported GvHD or protocol-defined DLT events. As shown in FIG. 7, the best ORR was 78% (7/9), and the complete response (CR) rate was 78% (7/9). For the four patients who had prior CD19 CAR T therapies, the ORR was 100% (4/4) and CR rate was also 100%. As of the data cut-off date, of the seven patients who had achieved CR, two patients progressed, one died while in complete remission and four were still in CR and in active follow-up, with a range of follow-up time between 1.2 and 8.8 months. CAR+γδ T cell kinetics improved in a dose-dependent manner with peak cell expansion occurring between Days 7 and 10 at DL3 based on flow cytometry.

Conclusions-ADI-001 γδ CAR T cells maintained a favorable safety profile. Preliminary efficacy showed encouraging CR rate and sustained durability in patients, including those previously exposed to CAR T therapy.

Sequence S information as herein disclosed is summarized below in Table 3.

TABLE 3
Sequence Information

| SEQ | | |
|---|---|---|
| ID | | |
| No. | Name | Sequence |
| 1 | HCDR3 | AKDPSYGSGSYHSYYGMDV |
| | polypeptide | |
| 2 | LCDR3 | QQRFNWPLT |
| | polypeptide | |
| 3 | HCDR3 | VKDFHYGSGSNYGMDV |
| | polypeptide | |
| 4 | LCDR3 | QQSNDWPLT |
| | polypeptide | |
| 5 | HCDR3 | TKDGSYGHFYSGLDV |
| | polypeptide | |
| 6 | LCDR3 | QQRYYWPLT |
| | polypeptide | |
| 7 | HCVR | EEQLVESGGDLVQPGRSLRLSCAAS |
| | polypeptide | GFTFHDYTMHWVRQAPGKGLEWVSG |
| | | ISWNSGSLGYADSVKGRFTISRDNA |
| | | KKSLYLQMNSLRAEDTALYYCAKDP |
| | | SYGSGSYHSYYGMDVWGQGTTVTVS |
| | | S |
| 8 | LCVR | EIVLTQSPATLSLSPGERATLSCWA |
| | polypeptide | SQSISRYLVWYQQKCGQAPRLLIYE |
| | | ASKRATGIPVRFSGSGSGTDFTLTI |
| | | SSLESEDFAVYYCQQRFNWPLTFGG |
| | | GTKVEIK |
| 9 | HCVR | EVQLAESGGDLVQSGRSLRLSCAAS |
| | polypeptide | GITFHDYAMHWVRQPPGKGLEWVSG |
| | | ISWNSDYIGYADSVKGRFTISRDNA |
| | | KKSLYLQMNSLRPDDTALYYCVKDF |
| | | HYGSGSNYGMDVWGQGTTVTVSP |
| 10 | LCVR | EIVMTQSPATLSMSPGERATLSCRA |
| | polypeptide | SQSVSRNLAWYQQKVGQAPRLLISG |
| | | ASTRATGIPARFSGSGSGTEFTLTI |
| | | NSLQSEDFAVYYCQQSNDWPLTFGQ |
| | | GTRLEIK |
| 11 | HCVR | EVQLVESGGGLVQPGRSLRLSCAAS |
| | polypeptide | GFTFYDYAMHWVRQAPGKGLEWVSG |
| | | ISWNSDTIGYADSVKGRFTISRDNA |
| | | KNSLYLQMNSLRAEDTALYYCTKDG |
| | | SYGHFYSGLDVWGQGTTVTVSS |
| 12 | LCVR | EIVLTQSPATLSLSPGERATLSCRA |
| | polypeptide | SQSVSSYLAWYQQKPGQAPRLLIYV |
| | | ASNRATGIPARFSGSGSGTDFTLTI |
| | | SSLEPDDFAVYYCQQRYYWPLTFGG |
| | | GTKVEIK |
| 13 | HCVR | EVQLVESGGGLVQPGRSLRLSCAAS |
| | polypeptide | GFTFYDYAMHWVRQAPGKGLEWVSG |
| | | ISWNSGYIGYADSVKGRFTISRDNA |
| | | KNSLYLQMNSLRAEDTALYYCAKDN |
| | | SYGKFYYGLDVWGQGTTVTVSS |
| 14 | LCVR | EIVMTQSPATLSVSPGERTTLSCRA |
| | polypeptide | SQSVSSNLAWYLQKPGQAPRLLIYG |
| | | ASTRATGIPARFSGSGSGTEFILTI |
| | | SSLQSEDFAVYYCQQYNNWPITFGQ |
| | | GTRLEIK |
| 15 | HCDR1 | GFTFYDYA |
| | polypeptide | |
| 16 | HCDR2 | ISWNSGYI |
| | polypeptide | |
| 17 | HCDR3 | AKDNSYGKFYYGLDV |
| | polypeptide | |
| 18 | LCDR1 | QSVSSN |
| | polypeptide | |
| 19 | LCDR2 | GAS |
| | polypeptide | |
| 20 | LCDR3 | QQYNNWPIT |
| | polypeptide | |
| 21 | CD8α hinge | PTPAPTIASQPLSLRPEACRPAAGG |
| | domain | AVHTRGLDFACDIY |
| | polypeptide | |
| 22 | CD8α hinge | TTTPAPRPPTPAPTIASQPLSLRPE |
| | domain | ACRPAAGGAVHTRGLDFACDIY |
| | polypeptide | |
| 23 | CD8α | IWAPLAGTCGVLLLSLVITLYC |
| | transmembrane | |
| | domain | |
| | polypeptide | |
| 24 | CD3ζ signaling | RVKFSRSADAPAYQQGQNQLYNELN |
| | domain | LGRREEYDVLDKRRGRDPEMGGKP |
| | polypeptide | QRRKNPQEGLYNELQKDKMAEAYSE |
| | | IGMKGERRRGKGDGLYQGLSTATKD |
| | | TYDALHMQALPPR |
| 25 | CD3ζ signaling | RVKFSRSADAPAYQQGQNQLYNELN |
| | domain | LGRREEYDVLDKRRGRDPEMGGKPR |
| | polypeptide | RKNPQEGLYNELQKDKMAEAYSEIG |
| | | MKGERRRGKGHDGLYQGLSTATKDT |
| | | YDALHMQALPPR |
| 26 | 4-1BB | KRGRKKLLYIFKQPFMRPVQTTQEE |
| | costimulatory | DGCSCRFPEEEEGGCEL |
| | domain | |
| | polypeptide | |
| 27 | CD27 | QRRKYRSNKGESPVEPAEPCHYSCP |
| | costimulatory | REEEGSTIPIQEDYRKPEPACSP |
| | domain | |
| | polypeptide | |
| 28 | secretion | MALPVTALLLPLALLLHAARP |
| | signal | |
| 29 | IL-15 | NWVNVISDLKKIEDLIQSMHIDATL |
| | polypeptide | YTESDVHPSCKVTAMKCFLLELQVI |
| | | SLESGDASIHDTVENLIILANNSLS |
| | | SNGNVTESGCKECEELEEKNIKEFL |
| | | QSFVHIVQMFINTS |
| 30 | P2A cleavage | SGSGATNFSLLKQAGDVEENPGP |
| | sequence | |
| | polypeptide | |
| 31 | furin cleavage | RAKR |
| | sequence | |
| | polypeptide | |
| 32 | P2A + furin | RAKRSGSGATNFSLLKQAGDVEEN |
| | cleavage | PGP |
| | sequence | |
| | polypeptide | |
| 33 | P2A cleavage | ATNFSLLKQAGDVEENPGP |
| | sequence | |
| | polypeptide | |
| 34 | F2A cleavage | VKQTLNNFDLLKLAGDVESNPGP |
| | sequence | |
| | polypeptide | |
| 35 | E2A cleavage | QCTNYALLKLAGDVESNPGP |
| | sequence | |
| | polypeptide | |
| 36 | T2A cleavage | EGRSLLTCGDVEENPGP |
| | sequence | |
| | polypeptide | |
| 37 | IRES | CTAACGTTACTGGCCGAAGCCGCTT |
| | | GGAATAAGGCCGGTGTGCGTTTGTC |
| | | TATATGTTATTTTCCACCATATTGC |
| | | CGTCTTTTGGCAATGTGAGGGCCCG |
| | | GAAACCTGGCCCTGTCTTCTTGACG |
| | | AGCATTCCTAGGGGTCTTTCCCCTC |
| | | TCGCCAAAGGAATGCAAGGTCTGTT |
| | | GAATGTCGTGAAGGAAGCAGTTCCT |
| | | CTGGAAGCTTCTTGAAGACAAACAA |
| | | CGTCTGTAGCGACCCTTTGCAGGCA |
| | | GCGGAACCCCCCACCTGGCGACAGG |
| | | TGCCTCTGCGGCCAAAAGCCACGTG |
| | | TATAAGATACACCTGCAAAGGCGGC |
| | | ACAACCCCAGTGCCACGTTGTGAGT |
| | | TGGATAGTTGTGGAAAGAGTCAAAT |
| | | GGCTCTCCTCAAGCGTATTCAACAA |
| | | GGGGCTGAAGGATGCCCAGAAGGTA |
| | | CCCCATTGTATGGGATCTGATCTGG |
| | | GGCCTCGGTGCACATGCTTTACATG |
| | | TGTTTAGTCGAGGTTAAAAAAACGT |
| | | CTAGGCCCCCCGAACCACGGGGACG |
| | | TGGTTTTCCTTTGAAAAACACGATG |
| | | ATA |
| 38 | IRES | AGCAGGTTTCCCCAACTGACACAAA |
| | | ACGTGCAACTTGAAACTCCGCCTGG |
| | | TCTTTCCAGGTCTAGAGGGGTAACA |
| | | CTTTGTACTGCGTTTGGCTCCACGC |
| | | TCGATCCACTGGCGAGTGTTAGTAA |
| | | CAGCACTGTTGCTTCGTAGCGGAGC |
| | | ATGACGGCCGTGGGAACTCCTCCTT |
| | | GGTAACAAGGACCCACGGGGCCAAA |
| | | AGCCACGCCCACACGGGCCCGTCAT |
| | | GTGTGCAACCCCAGCACGGCGACTT |
| | | TACTGCGAAACCCACTTTAAAGTGA |
| | | CATTGAAACTGGTACCCACACACTG |
| | | GTGACAGGCTAAGGATGCCCTTCAG |
| | | GTACCCCGAGGTAACACGCGACACT |
| | | CGGGATCTGAGAAGGGGACTGGGGC |
| | | TTCTATAAAAGCGCTCGGTTTAAAA |
| | | AGCTTCTATGCCTGAATAGGTGACC |
| | | GGAGGTCGGCACCTTTCCTTTGCAA |
| | | TTACTGACCAC |
| 39 | 3H7-CD8- | MSVPTQVLGLLLLWLTDARCEIVMT |
| | CD27z | QSPATLSVSPGERTTLSCRASQSVS |
| | polypeptide | SNLAWYLQKPGQAPRLLIYGASTRA |
| | | TGIPARFSGSGSGTEFILTISSLQS |
| | | EDFAVYYCQQYNNWPITFGQGTRLE |
| | | IKGGGGSGGGGSGGGGEVQLVESGG |
| | | GLVQPGRSLRLSCAASGFTFYDYAM |
| | | HWVRQAPGKGLEWVSGISWNSGYIG |
| | | YADSVKGRFTISRDNAKNSLYLQMN |
| | | SLRAEDTALYYCAKDNSYGKFYYGL |
| | | DVWGQGTTVTVSSTTTPAPRPPTPA |
| | | PTIASQPLSLRPEACRPAAGGAVHT |
| | | RGLDFACDIYIWAPLAGTCGVLLLS |
| | | LVITLYCQRRKYRSNKGESPVEPAE |
| | | PCHYSCPREEEGSTIPIQEDYRKPE |
| | | PACSPRVKFSRSADAPAYQQGQNQL |
| | | YNELNLGRREEYDVLDKRRGRDPEM |
| | | GGKPQRRKNPQEGLYNELQKDKMAE |
| | | AYSEIGMKGERRRGKGHDGLYQGLS |
| | | TATKDTYDALHMQALPPR |
| 40 | 3B9-CD8-BBz | MSVPTQVLGLLLLWLTDARCEIVMT |
| | polypeptide | QSPATLSVSPGERATLSCRASQSVS |
| | | SNLAWYQQKPGQAPRLLIYGTSTRA |
| | | TGIPARFSGSGSGTEFTLTISSLQS |
| | | EDFAVYYCQQYNNWPLTFGGGTKVE |
| | | IKGGGGSGGGGSGGGGEVQLVESGG |
| | | GLVQPGRSLRLSCVASGFTFNDYAM |
| | | HWVRQAPGKGLEWVSVISWNSDSIG |
| | | YADSVKGRFTISRDNAKNSLYLQMH |
| | | SLRAEDTALYYCAKDNHYGSGSYYY |
| | | YQYGMDVWGQGTTVTVSSTTTPAPR |
| | | PPTPAPTIASQPLSLRPEACRPAAG |
| | | GAVHTRGLDFACDIYIWAPLAGTCG |
| | | VLLLSLVITLYCKRGRKKLLYIFKQ |
| | | PFMRPVQTTQEEDGCSCRFPEEEEG |
| | | GCELRVKFSRSADAPAYQQGQNQLY |
| | | NELNLGRREEYDVLDKRRGRDPEMG |
| | | GKPQRRKNPQEGLYNELQKDKMAEA |
| | | YSEIGMKGERRRGKGHDGLYQGLST |
| | | ATKDTYDALHMQALPPR |
| 41 | 3H7-CD8-BBz | MSVPTQVLGLLLLWLTDARCEIVMT |
| | polypeptide | QSPATLSVSPGERTTLSCRASQSVS |
| | | SNLAWYLQKPGQAPRLLIYGASTRA |
| | | TGIPARFSGSGSGTEFILTISSLQS |
| | | EDFAVYYCQQYNNWPITFGQGTRLE |
| | | IKGGGGSGGGGSGGGGEVQLVESGG |
| | | GLVQPGRSLRLSCAASGFTFYDYAM |
| | | HWVRQAPGKGLEWVSGISWNSGYIG |
| | | YADSVKGRFTISRDNAKNSLYLQMN |
| | | SLRAEDTALYYCAKDNSYGKFYYGL |
| | | DVWGQGTTVTVSSTTTPAPRPPTPA |
| | | PTIASQPLSLRPEACRPAAGGAVHT |
| | | RGLDFACDIYIWAPLAGTCGVLLLS |
| | | LVITLYCKRGRKKLLYIFKQPFMRP |
| | | VQTTQEEDGCSCRFPEEEEGGCELR |
| | | VKFSRSADAPAYQQGQNQLYNELNL |
| | | GRREEYDVLDKRRGRDPEMGGKPQR |
| | | RKNPQEGLYNELQKDKMAEAYSEIG |
| | | MKGERRRGKGHDGLYQGLSTATKDT |
| | | YDALHMQALPPR |
| 42 | 2B7-CD8-BBz | MSVPTQVLGLLLLWLTDARCEIVLT |
| | polypeptide | QSPATLSLSPGERAALSCRASQSVS |
| | | NYLAWYQQKPGQAPRLLIYDASNRA |
| | | TGIPARFSGSGSGTDFTLTISSLEP |
| | | EDFAVYYCQQRSNWPLTFGGGTKVE |
| | | IRGGGGSGGGGSGGGGEVQLVESGG |
| | | GLVQPGRSLRLSCAASGFTFRDYTM |
| | | HWVRQGPGKGLEWVSGISWNSDYIG |
| | | YADSVKGRFTISRDNAKNSLYLQMN |
| | | SLRVEDTALYYCAKLSGTYRDYFYG |
| | | VDVWGQGTTVTVSSTTTPAPRPPTP |
| | | APTIASQPLSLRPEACRPAAGGAVH |
| | | TRGLDFACDIYIWAPLAGTCGVLLL |
| | | SLVITLYCKRGRKKLLYIFKQPFMR |
| | | PVQTTQEEDGCSCRFPEEEEGGCEL |
| | | RVKFSRSADAPAYQQGQNQLYNELN |
| | | LGRREEYDVLDKRRGRDPEMGGKPQ |
| | | RRKNPQEGLYNELQKDKMAEAYSEI |
| | | GMKGERRRGKGHDGLYQGLSTATKD |
| | | TYDALHMQALPPR |
| 43 | 9C11-CD8- | MSVPTQVLGLLLLWLTDARCEIVVT |
| | BBz | QSPATLSLSPGERATLSCRTSQTTT |
| | polypeptide | SYLAWYRQKPGQAPRLLIYDASNRA |
| | | AGIPARFSGSGSGTDFTLTINSLEP |
| | | EDFAVYYCQLRTNWITFGQGTRLEI |
| | | KGGGGSGGGGSGGGGQVQLVESGGD |
| | | SVKPGGSLRLSCAASGFTFSDSYMT |
| | | WIRQAPGKGLEWVSFISSSGSTIYY |
| | | ADSVKGRFTISRDNVKKSLYLQMNR |
| | | LRAEDTAVYYCAREEPGNYVYYGMD |
| | | VWGQGTTVTVSSTTTPAPRPPTPAP |
| | | TIASQPLSLRPEACRPAAGGAVHTR |
| | | GLDFACDIYIWAPLAGTCGVLLLSL |
| | | VITLYCKRGRKKLLYIFKQPFMRPV |
| | | QTTQEEDGCSCRFPEEEEGGCELRV |
| | | KFSRSADAPAYQQGQNQLYNELNLG |
| | | RREEYDVLDKRRGRDPEMGGKPQRR |
| | | KNPQEGLYNELQKDKMAEAYSEIGM |
| | | KGERRRGKGHDGLYQGLSTATKDTY |
| | | DALHMQALPPR |
| 44 | 3H7-CD3z | M S V P T Q V L G L L L |
| | polypeptide | L W L T D A R C E I V M |
| | | T Q S P A T L S V S P G |
| | | E R T T L S C R A S Q S |
| | | V S S N L A W Y L Q K P |
| | | G Q A P R L L I Y G A S |
| | | T R A T G I P A R F S G |
| | | S G S G T E F I L T I S |
| | | S L Q S E D F A V Y Y C |
| | | Q Q Y N N W P I T F G Q |
| | | G T R L E I K G G G G S |
| | | G G G G S G G G G E V Q |
| | | L V E S G G G L V Q P G |
| | | R S L R L S C A A S G F |
| | | T F Y D Y A M H W V R Q |
| | | A P G K G L E W V S G I |
| | | S W N S G Y I G Y A D S |
| | | V K G R F T I S R D N A |
| | | K N S L Y L Q M N S L R |
| | | A E D T A L Y Y C A K D |
| | | N S Y G K F Y Y G L D V |
| | | W G Q G T T V T V S S T |
| | | T T P A P R P P T P A P |
| | | T I A S Q P L S L R P E |
| | | A C R P A A G G A V H T |
| | | R G L D F A C D I Y I W |
| | | A P L A G T C G V L L L |
| | | S L V I T L Y C R V K F |
| | | S R S A D A P A Y Q Q G |
| | | Q N Q L Y N E L N L G R |
| | | R E E Y D V L D K R R G |
| | | R D P E M G G K P Q R R |
| | | K N P Q E G L Y N E L Q |
| | | K D K M A E A Y S E I G |
| | | M K G E R R R G K G H D |
| | | G L Y Q G L S T A T K D |
| | | T Y D A L H M Q A L P P |
| | | R |
| 45 | 3H7-CD8-27z | ATGTCCGTGCCTACCCAGGTGCTGG |
| | nucleotide | GCCTGCTGCTGCTGTGGCTGACCGA |
| | | CGCCAGATGCGAAATAGTGATGACG |
| | | CAGTCTCCAGCCACCCTGTCTGTGT |
| | | CTCCAGGGGAAAGAACCACCCTCTC |
| | | CTGCAGGGCCAGTCAGAGTGTTAGC |
| | | AGCAACTTAGCCTGGTACCTTCAGA |
| | | AACCTGGCCAGGCTCCCAGGCTCCT |
| | | CATCTATGGTGCATCCACCAGGGCC |
| | | ACTGGTATCCCAGCCAGGTTCAGTG |
| | | GCAGTGGGTCTGGGACAGAGTTCAT |
| | | TCTCACCATCAGCAGCCTGCAGTCT |
| | | GAAGATTTTGCAGTTTATTACTGTC |
| | | AGCAGTATAATAACTGGCCGATCAC |
| | | CTTCGGCCAAGGGACACGGCTGGAG |
| | | ATTAAAGGTGGAGGTGGATCTGGAG |
| | | GAGGAGGATCCGGTGGAGGAGGTGA |
| | | AGTGCAACTGGTGGAGTCTGGGGGA |
| | | GGCTTGGTACAGCCTGGCAGGTCCC |
| | | TGAGACTCTCCTGTGCAGCCTCTGG |
| | | ATTCACCTTTTATGATTATGCCATG |
| | | CACTGGGTCCGGCAAGCTCCAGGGA |
| | | AGGGCCTGGAGTGGGTCTCAGGTAT |
| | | TAGTTGGAATAGTGGTTACATAGGC |
| | | TATGCGGACTCTGTGAAGGGCCGAT |
| | | TCACCATCTCCAGAGACAACGCCAA |
| | | GAACTCCCTGTATCTGCAAATGAAC |
| | | AGTCTGAGAGCTGAGGACACGGCCT |
| | | TGTATTACTGTGCAAAAGATAACAG |
| | | CTATGGAAAGTTCTACTACGGTTTG |
| | | GACGTCTGGGGCCAAGGGACCACGG |
| | | TCACCGTCTCCTCAACCACGACGCC |
| | | AGCGCCGCGACCACCAACACCGGCG |
| | | CCCACCATCGCGTCGCAGCCCCTGT |
| | | CCCTGCGCCCAGAGGCGTGCCGGCC |
| | | AGCGGCGGGGGGCGCAGTGCACACG |
| | | AGGGGGCTGGACTTCGCCTGTGATA |
| | | TCTACATCTGGGCGCCCTTGGCCGG |
| | | GACTTGTGGGGTCCTTCTCCTGTCA |
| | | CTGGTTATCACCCTTTACTGCCAAC |
| | | GACGCAAGTACCGCTCCAATAAAGG |
| | | AGAGTCACCAGTAGAACCCGCCGAA |
| | | CCTTGTCACTATTCATGTCCACGCG |
| | | AAGAGGAGGGTTCAACGATCCCTAT |
| | | TCAGGAAGATTACAGAAAGCCGGAA |
| | | CCTGCTTGTAGCCCCAGAGTGAAGT |
| | | TCAGCCGCAGCGCCGACGCCCCTGC |
| | | CTACCAGCAGGGCCAGAACCAGCTG |
| | | TATAACGAGCTGAACCTGGGCAGGC |
| | | GGGAGGAATACGACGTGCTGGACAA |
| | | GCGCAGAGGCCGGGACCCTGAGATG |
| | | GGCGGCAAGCCCCAGAGGCGGAAGA |
| | | ACCCCCAGGAAGGCCTGTATAACGA |
| | | ACTGCAGAAAGACAAGATGGCCGAG |
| | | GCCTACAGCGAGATCGGCATGAAGG |
| | | GCGAGCGGCGACGCGGCAAGGGCCA |
| | | CGACGGCCTGTACCAGGGCCTGTCC |
| | | ACCGCCACCAAGGACACCTACGACG |
| | | CCCTGCACATGCAGGCCCTGCCTCC |
| | | CCGTTAG |
| 46 | 3H7-CD8-BBz | ATGTCCGTGCCTACCCAGGTGCTGG |
| | nucleotide | GCCTGCTGCTGCTGTGGCTGACCGA |
| | | CGCCAGATGCGAAATAGTGATGACG |
| | | CAGTCTCCAGCCACCCTGTCTGTGT |
| | | CTCCAGGGGAAAGAACCACCCTCTC |
| | | CTGCAGGGCCAGTCAGAGTGTTAGC |
| | | AGCAACTTAGCCTGGTACCTTCAGA |
| | | AACCTGGCCAGGCTCCCAGGCTCCT |
| | | CATCTATGGTGCATCCACCAGGGCC |
| | | ACTGGTATCCCAGCCAGGTTCAGTG |
| | | GCAGTGGGTCTGGGACAGAGTTCAT |
| | | TCTCACCATCAGCAGCCTGCAGTCT |
| | | GAAGATTTTGCAGTTTATTACTGTC |
| | | AGCAGTATAATAACTGGCCGATCAC |
| | | CTTCGGCCAAGGGACACGGCTGGAG |
| | | ATTAAAGGTGGAGGTGGATCTGGAG |
| | | GAGGAGGATCCGGTGGAGGAGGTGA |
| | | AGTGCAACTGGTGGAGTCTGGGGGA |
| | | GGCTTGGTACAGCCTGGCAGGTCCC |
| | | TGAGACTCTCCTGTGCAGCCTCTGG |
| | | ATTCACCTTTTATGATTATGCCATG |
| | | CACTGGGTCCGGCAAGCTCCAGGGA |
| | | AGGGCCTGGAGTGGGTCTCAGGTAT |
| | | TAGTTGGAATAGTGGTTACATAGGC |
| | | TATGCGGACTCTGTGAAGGGCCGAT |
| | | TCACCATCTCCAGAGACAACGCCAA |
| | | GAACTCCCTGTATCTGCAAATGAAC |
| | | AGTCTGAGAGCTGAGGACACGGCCT |
| | | TGTATTACTGTGCAAAAGATAACAG |
| | | CTATGGAAAGTTCTACTACGGTTTG |
| | | GACGTCTGGGGCCAAGGGACCACGG |
| | | TCACCGTCTCCTCAACCACGACGCC |
| | | AGCGCCGCGACCACCAACACCGGCG |
| | | CCCACCATCGCGTCGCAGCCCCTGT |
| | | CCCTGCGCCCAGAGGCGTGCCGGCC |
| | | AGCGGCGGGGGGCGCAGTGCACACG |
| | | AGGGGGCTGGACTTCGCCTGTGATA |
| | | TCTACATCTGGGCGCCCTTGGCCGG |
| | | GACTTGTGGGGTCCTTCTCCTGTCA |
| | | CTGGTTATCACCCTTTACTGCAAAC |
| | | GGGGCAGAAAGAAACTCCTGTATAT |
| | | ATTCAAACAACCATTTATGAGACCA |
| | | GTACAAACTACTCAAGAGGAAGATG |
| | | GCTGTAGCTGCCGATTTCCAGAAGA |
| | | AGAAGAAGGAGGATGTGAACTGAGA |
| | | GTGAAGTTCAGCAGGAGCGCAGACG |
| | | CCCCCGCGTACCAGCAGGGCCAGAA |
| | | CCAGCTCTATAACGAGCTCAATCTA |
| | | GGACGAAGAGAGGAGTACGATGTTT |
| | | TGGACAAGAGACGTGGCCGGGACCC |
| | | TGAGATGGGGGGAAAGCCGCAGAGA |
| | | AGGAAGAACCCTCAGGAAGGCCTGT |
| | | ACAATGAACTGCAGAAAGATAAGAT |
| | | GGCGGAGGCCTACAGTGAGATTGGG |
| | | ATGAAAGGCGAGCGCCGGAGGGGCA |
| | | AGGGGCACGATGGCCTTTACCAGGG |
| | | TCTCAGTACAGCCACCAAGGACACC |
| | | TACGACGCCCTTCACATGCAGGCCC |
| | | TGCCCCCTCGCTAA |
| 47 | 3B9-CD8-BBz | ATGAGCGTTCCAACCCAAGTTCTGG |
| | nucleotide | GACTGCTTCTGCTCTGGTTGACTGA |
| | | CGCTAGGTGCGAAATAGTAATGACC |
| | | CAATCCCCAGCCACTCTCTCCGTTA |
| | | GCCCAGGTGAAAGAGCCACTCTTAG |
| | | TTGCAGGGCTAGTCAATCCGTATCT |
| | | AGCAACCTGGCCTGGTACCAGCAAA |
| | | AGCCCGGACAAGCGCCGCGGTTGTT |
| | | GATCTATGGGACGAGCACACGAGCT |
| | | ACGGGTATTCCGGCCAGGTTCTCAG |
| | | GGTCTGGCTCCGGAACCGAATTTAC |
| | | ATTGACGATCAGTAGTCTGCAATCA |
| | | GAGGATTTCGCCGTTTACTATTGCC |
| | | AACAGTACAATAATTGGCCGCTCAC |
| | | ATTCGGGGGAGGAACCAAGGTCGAG |
| | | ATTAAGGGAGGTGGGGGTAGTGGGG |
| | | GGGGGGGGTCAGGAGGTGGAGGAGA |
| | | GGTACAGTTGGTAGAAAGCGGCGGG |
| | | GGGTTGGTTCAACCTGGACGGAGTC |
| | | TGAGATTGTCTTGCGTGGCTTCCGG |
| | | CTTTACTTTCAATGATTACGCCATG |
| | | CACTGGGTACGCCAGGCGCCTGGAA |
| | | AGGGTCTGGAGTGGGTTTCCGTGAT |
| | | ATCCTGGAATAGTGATAGTATAGGC |
| | | TATGCCGATAGTGTAAAAGGAAGGT |
| | | TTACAATCTCTAGGGATAACGCTAA |
| | | GAACAGCCTGTACCTTCAAATGCAT |
| | | AGTCTCCGGGCTGAGGACACAGCCT |
| | | TGTACTATTGTGCTAAGGACAATCA |
| | | TTATGGAAGCGGGTCATATTATTAC |
| | | TATCAATATGGGATGGATGTGTGGG |
| | | GTCAGGGAACGACCGTTACGGTATC |
| | | CTCAACCACCACCCCTGCACCAAGG |
| | | CCCCCGACTCCCGCGCCCACCATCG |
| | | CGTCACAGCCTCTTAGCCTGCGACC |
| | | GGAAGCATGCAGACCAGCTGCCGGG |
| | | GGGGCCGTGCATACGAGAGGTTTGG |
| | | ACTTCGCCTGCGATATCTACATCTG |
| | | GGCGCCCTTGGCCGGGACTTGTGGG |
| | | GTCCTTCTCCTGTCACTGGTTATCA |
| | | CCCTTTACTGCAAACGGGGCAGAAA |
| | | GAAACTCCTGTATATATTCAAACAA |
| | | CCATTTATGAGACCAGTACAAACTA |
| | | CTCAAGAGGAAGATGGCTGTAGCTG |
| | | CCGATTTCCAGAAGAAGAAGAAGGA |
| | | GGATGTGAACTGAGAGTGAAGTTCA |
| | | GCAGGAGCGCAGACGCCCCCGCGTA |
| | | CCAGCAGGGCCAGAACCAGCTCTAT |
| | | AACGAGCTCAATCTAGGACGAAGAG |
| | | AGGAGTACGATGTTTTGGACAAGAG |
| | | ACGTGGCCGGGACCCTGAGATGGGG |
| | | GGAAAGCCGCAGAGAAGGAAGAACC |
| | | CTCAGGAAGGCCTGTACAATGAACT |
| | | GCAGAAAGATAAGATGGCGGAGGCC |
| | | TACAGTGAGATTGGGATGAAAGGCG |
| | | AGCGCCGGAGGGGCAAGGGGCACGA |
| | | TGGCCTTTACCAGGGTCTCAGTACA |
| | | GCCACCAAGGACACCTACGACGCCC |
| | | TTCACATGCAGGCCCTGCCCCCTCG |
| | | CTAA |
| 48 | 2B7-CD8-BBz | ATGTCCGTACCTACCCAGGTGCTGG |
| | nucleotide | GCCTGCTGCTGCTGTGGCTGACCGA |
| | | CGCCAGATGCGAAATTGTGTTGACA |
| | | CAGTCTCCAGCCACCCTGTCTTTGT |
| | | CTCCAGGGGAAAGAGCCGCCCTCTC |
| | | CTGCAGGGCCAGTCAGAGTGTTAGC |
| | | AACTACTTAGCCTGGTACCAACAGA |
| | | AACCTGGCCAGGCTCCCAGGCTCCT |
| | | CATCTATGATGCATCCAACAGGGCC |
| | | ACTGGCATCCCAGCCAGGTTCAGTG |
| | | GCAGTGGGTCTGGGACAGACTTCAC |
| | | TCTCACCATCAGCAGCCTAGAGCCT |
| | | GAAGATTTTGCAGTTTATTACTGTC |
| | | AGCAGCGTAGCAACTGGCCGCTCAC |
| | | TTTCGGCGGAGGGACCAAGGTGGAG |
| | | ATCAGAGGTGGAGGTGGATCTGGAG |
| | | GAGGAGGATCCGGTGGAGGAGGTGA |
| | | AGTGCAGCTGGTGGAGTCTGGGGGA |
| | | GGCTTGGTACAGCCTGGCAGGTCCC |
| | | TGCGACTCTCCTGTGCAGCCTCTGG |
| | | ATTCACCTTTCGAGATTATACCATG |
| | | CACTGGGTCCGGCAAGGTCCAGGGA |
| | | AGGGCCTGGAATGGGTCTCAGGTAT |
| | | TAGTTGGAATAGTGATTACATAGGC |
| | | TATGCGGACTCTGTGAAGGGCCGAT |
| | | TCACCATCTCCAGAGACAACGCCAA |
| | | GAACTCCCTGTATCTGCAAATGAAC |
| | | AGTCTGAGAGTTGAGGACACGGCCT |
| | | TGTATTACTGTGCAAAGCTCAGTGG |
| | | GACCTACAGGGACTACTICTACGGA |
| | | GTGGACGTCTGGGGCCAAGGGACCA |
| | | CGGTCACCGTCTCCTCAACCACCAC |
| | | CCCTGCACCAAGGCCCCCGACTCCC |
| | | GCGCCCACCATCGCGTCACAGCCTC |
| | | TTAGCCTGCGACCGGAAGCATGCAG |
| | | ACCAGCTGCCGGGGGGGCCGTGCAT |
| | | ACGAGAGGTTTGGACTTCGCCTGCG |
| | | ATATCTACATCTGGGCGCCCTTGGC |
| | | CGGGACTTGTGGGGTCCTTCTCCTG |
| | | TCACTGGTTATCACCCTTTACTGCA |
| | | AACGGGGCAGAAAGAAACTCCTGTA |
| | | TATATTCAAACAACCATTTATGAGA |
| | | CCAGTACAAACTACTCAAGAGGAAG |
| | | ATGGCTGTAGCTGCCGATTTCCAGA |
| | | AGAAGAAGAAGGAGGATGTGAACTG |
| | | AGAGTGAAGTTCAGCAGGAGCGCAG |
| | | ACGCCCCCGCGTACCAGCAGGGCCA |
| | | GAACCAGCTCTATAACGAGCTCAAT |
| | | CTAGGACGAAGAGAGGAGTACGATG |
| | | TTTTGGACAAGAGACGTGGCCGGGA |
| | | CCCTGAGATGGGGGGAAAGCCGCAG |
| | | AGAAGGAAGAACCCTCAGGAAGGCC |
| | | TGTACAATGAACTGCAGAAAGATAA |
| | | GATGGCGGAGGCCTACAGTGAGATT |
| | | GGGATGAAAGGCGAGCGCCGGAGGG |
| | | GCAAGGGGCACGATGGCCTTTACCA |
| | | GGGTCTCAGTACAGCCACCAAGGAC |
| | | ACCTACGACGCCCTTCACATGCAGG |
| | | CCCTGCCCCCTCGCTAA |
| 49 | 9C11-CD8- | ATGTCCGTGCCTACCCAGGTGCTGG |
| | BBz nucleotide | GCCTGCTGCTGCTGTGGCTGACCGA |
| | | CGCCAGATGCGAAATTGTGGTGACA |
| | | CAGTCTCCAGCCACCCTGTCTTTGT |
| | | CTCCAGGGGAAAGAGCCACCCTCTC |
| | | CTGCAGGACCAGTCAGACTACTACC |
| | | AGCTACTTAGCCTGGTACCGACAGA |
| | | AACCTGGCCAGGCTCCCAGGCTCCT |
| | | CATCTATGATGCATCCAACAGGGCC |
| | | GCTGGCATCCCAGCCAGGTTCAGTG |
| | | GCAGTGGGTCTGGGACAGACTTCAC |
| | | TCTCACCATCAACAGCCTGGAGCCT |
| | | GAAGATTTTGCAGTTTATTACTGTC |
| | | AGCTGCGTACCAACTGGATCACCTT |
| | | CGGCCAAGGGACACGACTGGAGATT |
| | | AAAGGTGGAGGTGGATCTGGAGGAG |
| | | GAGGATCCGGTGGAGGAGGTCAGGT |
| | | GCAGCTGGTGGAGTCTGGGGGAGAC |
| | | TCGGTCAAGCCTGGAGGGTCCCTGA |
| | | GACTCTCCTGTGCAGCCTCTGGATT |
| | | CACCTTCAGTGACTCCTACATGACT |
| | | TGGATCCGCCAGGCTCCAGGGAAGG |
| | | GGCTGGAGTGGGTTTCATTCATTAG |
| | | TAGTAGTGGAAGTACCATATATTAT |
| | | GCAGACTCTGTGAAGGGCCGATTCA |
| | | CCATTTCCAGGGACAACGTCAAGAA |
| | | GTCATTGTATCTGCAGATGAACAGA |
| | | CTGAGAGCCGAGGACACGGCCGTGT |
| | | ATTACTGTGCGAGAGAAGAACCAGG |
| | | AAACTACGTCTATTACGGTATGGAC |
| | | GTCTGGGGCCAAGGGACCACGGTCA |
| | | CCGTCTCCTCAACCACCACCCCTGC |
| | | ACCAAGGCCCCCGACTCCCGCGCCC |
| | | ACCATCGCGTCACAGCCTCTTAGCC |
| | | TGCGACCGGAAGCATGCAGACCAGC |
| | | TGCCGGGGGGGCCGTGCATACGAGA |
| | | GGTTTGGACTTCGCCTGCGATATCT |
| | | ACATCTGGGCGCCCTTGGCCGGGAC |
| | | TTGTGGGGTCCTTCTCCTGTCACTG |
| | | GTTATCACCCTTTACTGCAAACGGG |
| | | GCAGAAAGAAACTCCTGTATATATT |
| | | CAAACAACCATTTATGAGACCAGTA |
| | | CAAACTACTCAAGAGGAAGATGGCT |
| | | GTAGCTGCCGATTTCCAGAAGAAGA |
| | | AGAAGGAGGATGTGAACTGAGAGTG |
| | | AAGTTCAGCAGGAGCGCAGACGCCC |
| | | CCGCGTACCAGCAGGGCCAGAACCA |
| | | GCTCTATAACGAGCTCAATCTAGGA |
| | | CGAAGAGAGGAGTACGATGTTTTGG |
| | | ACAAGAGACGTGGCCGGGACCCTGA |
| | | GATGGGGGGAAAGCCGCAGAGAAGG |
| | | AAGAACCCTCAGGAAGGCCTGTACA |
| | | ATGAACTGCAGAAAGATAAGATGGC |
| | | GGAGGCCTACAGTGAGATTGGGATG |
| | | AAAGGCGAGCGCCGGAGGGGCAAGG |
| | | GGCACGATGGCCTTTACCAGGGTCT |
| | | CAGTACAGCCACCAAGGACACCTAC |
| | | GACGCCCTTCACATGCAGGCCCTGC |
| | | CCCCTCGCTAA |
| 50 | Codon- | ACCACCACCCCTGCACCAAGGCCCC |
| | optimized | CGACTCCCGCGCCCACCATCGCGTC |
| | CD8α hinge | ACAGCCTCTTAGCCTGCGACCGGAA |
| | region | GCATGCAGACCAGCTGCCGGGGGGG |
| | nucleotide | CCGTGCATACGAGAGGTTTGGACTT |
| | | CGCCTGCGAT |
| 51 | Codon- | ACCACGACGCCAGCGCCGCGACCAC |
| | optimized | CAACACCGGCGCCCACCATCGCGTC |
| | CD8α hinge | GCAGCCCCTGTCCCTGCGCCCAGAG |
| | region | GCGTGCCGGCCAGCGGCGGGGGGCG |
| | nucleotide | CAGTGCACACGAGGGGGCTGGACTT |
| | | CGCCTGTGAT |
| 52 | anti-CD20- | MSVPTQVLGLLLLWLTDARCEIVMT |
| | CAR | QSPATLSVSPGERTTLSCRASQSVS |
| | polypeptide | SNLAWYLQKPGQAPRLLIYGASTRA |
| | | TGIPARFSGSGSGTEFILTISSLQS |
| | | EDFAVYYCQQYNNWPITFGQGTRLE |
| | | IKGGGGSGGGGSGGGGEVQLVESGG |
| | | GLVQPGRSLRLSCAASGFTFYDYAM |
| | | HWVRQAPGKGLEWVSGISWNSGYIG |
| | | YADSVKGRFTISRDNAKNSLYLQMN |
| | | SLRAEDTALYYCAKDNSYGKFYYGL |
| | | DVWGQGTTVTVSSTTTPAPRPPTPA |
| | | PTIASQPLSLRPEACRPAAGGAVHT |
| | | RGLDFACDIYIWAPLAGTCGVLLLS |
| | | LVITLYCKRGRKKLLYIFKQPFMRP |
| | | VQTTQEEDGCSCRFPEEEEGGCELR |
| | | VKFSRSADAPAYQQGQNQLYNELNL |
| | | GRREEYDVLDKRRGRDPEMGGKPQR |
| | | RKNPQEGLYNELQKDKMAEAYSEIG |
| | | MKGERRRGKGHDGLYQGLSTATKDT |
| | | YDALHMQALPPRGSGATNFSLLKQA |
| | | GDVEENPGPMALPVTALLLPLALLL |
| | | HAARPNWVNVISDLKKIEDLIQSMH |
| | | IDATLYTESDVHPSCKVTAMKCFLL |
| | | ELQVISLESGDASIHDTVENLIILA |
| | | NNSLSSNGNVTESGCKECEELEEKN |
| | | IKEFLQSFVHIVQMFINTS* |
| 53 | P2A cleavage | GSGATNFSLLKQAGDVEENPGP |
| | domain | |
| | polypeptide | |
| 54 | anti-CD20- | MSVPTQVLGLLLLWLTDARCEIVMT |
| | CAR | QSPATLSVSPGERTTLSCRASQSVS |
| | polypeptide | SNLAWYLQKPGQAPRLLIYGASTRA |
| | | TGIPARFSGSGSGTEFILTISSLQS |
| | | EDFAVYYCQQYNNWPITFGQGTRLE |
| | | IKGGGGSGGGGSGGGGEVQLVESGG |
| | | GLVQPGRSLRLSCAASGFTFYDYAM |
| | | HWVRQAPGKGLEWVSGISWNSGYIG |
| | | YADSVKGRFTISRDNAKNSLYLQMN |
| | | SLRAEDTALYYCAKDNSYGKFYYGL |
| | | DVWGQGTTVTVSSTTTPAPRPPTPA |
| | | PTIASQPLSLRPEACRPAAGGAVHI |
| | | RGLDFACDIYIWAPLAGTCGVLLLS |
| | | LVITLYCKRGRKKLLYIFKQPFMRP |
| | | VQTTQEEDGCSCRFPEEEEGGCELR |
| | | VKFSRSADAPAYQQGQNQLYNELNL |
| | | GRREEYDVLDKRRGRDPEMGGKPQR |
| | | RKNPQEGLYNELQKDKMAEAYSEIG |
| | | MKGERRRGKGHDGLYQGLSTATKDT |
| | | YDALHMQALPPRGSGATNFSLLKQA |
| | | GDVEENPGPMRISKPHLRSISIQCY |
| | | LCLLLNSHFLTEAGIHVFILGCFSA |
| | | GLPKTEANWVNVISDLKKIEDLIQS |
| | | MHIDATLYTESDVHPSCKVTAMKCF |
| | | LLELQVISLESGDASIHDTVENLII |
| | | LANNSLSSNGNVTESGCKECEELEE |
| | | KNIKEFLQSFVHIVQMFINTS* |
| 55 | Secretion signal | MRISKPHLRSISIQCYLCLLLNSHF |
| | polypeptide | LTEAGIHVFILGCFSAGLPKTEA |
| 56 | anti-CD20 | ATGTCCGTGCCTACCCAGGTGCTGG |
| | CAR + sIL 15 | GCCTGCTGCTGCTGTGGCTGACCGA |
| | nucleotide | CGCCAGATGCGAAATAGTGATGACG |
| | | CAGTCTCCAGCCACCCTGTCTGTGT |
| | | CTCCAGGGGAAAGAACCACCCTCTC |
| | | CTGCAGGGCCAGTCAGAGTGTTAGC |
| | | AGCAACTTAGCCTGGTACCTTCAGA |
| | | AACCTGGCCAGGCTCCCAGGCTCCT |
| | | CATCTATGGTGCATCCACCAGGGCC |
| | | ACTGGTATCCCAGCCAGGTTCAGTG |
| | | GCAGTGGGTCTGGGACAGAGTTCAT |
| | | TCTCACCATCAGCAGCCTGCAGTCT |
| | | GAAGATTTTGCAGTTTATTACTGTC |
| | | AGCAGTATAATAACTGGCCGATCAC |
| | | CTTCGGCCAAGGGACACGGCTGGAG |
| | | ATTAAAGGTGGAGGTGGATCTGGAG |
| | | GAGGAGGATCCGGTGGAGGAGGTGA |
| | | AGTGCAACTGGTGGAGTCTGGGGGA |
| | | GGCTTGGTACAGCCTGGCAGGTCCC |
| | | TGAGACTCTCCTGTGCAGCCTCTGG |
| | | ATTCACCTTTTATGATTATGCCATG |
| | | CACTGGGTCCGGCAAGCTCCAGGGA |
| | | AGGGCCTGGAGTGGGTCTCAGGTAT |
| | | TAGTTGGAATAGTGGTTACATAGGC |
| | | TATGCGGACTCTGTGAAGGGCCGAT |
| | | TCACCATCTCCAGAGACAACGCCAA |
| | | GAACTCCCTGTATCTGCAAATGAAC |
| | | AGTCTGAGAGCTGAGGACACGGCCT |
| | | TGTATTACTGTGCAAAAGATAACAG |
| | | CTATGGAAAGTTCTACTACGGTTTG |
| | | GACGTCTGGGGCCAAGGGACCACGG |
| | | TCACCGTCTCCTCAACCACGACGCC |
| | | AGCGCCGCGACCACCAACACCGGCG |
| | | CCCACCATCGCGTCGCAGCCCCTGT |
| | | CCCTGCGCCCAGAGGCGTGCCGGCC |
| | | AGCGGCGGGGGGCGCAGTGCACACG |
| | | AGGGGGCTGGACTTCGCCTGTGATA |
| | | TCTACATCTGGGCGCCCTTGGCCGG |
| | | GACTTGTGGGGTCCTTCTCCTGTCA |
| | | CTGGTTATCACCCTTTACTGCAAAC |
| | | GGGGCAGAAAGAAACTCCTGTATAT |
| | | ATTCAAACAACCATTTATGAGACCA |
| | | GTACAAACTACTCAAGAGGAAGATG |
| | | GCTGTAGCTGCCGATTTCCAGAAGA |
| | | AGAAGAAGGAGGATGTGAACTGAGA |
| | | GTGAAGTTCAGCAGGAGCGCAGACG |
| | | CCCCCGCGTACCAGCAGGGCCAGAA |
| | | CCAGCTCTATAACGAGCTCAATCTA |
| | | GGACGAAGAGAGGAGTACGATGTTT |
| | | TGGACAAGAGACGTGGCCGGGACCC |
| | | TGAGATGGGGGGAAAGCCGCAGAGA |
| | | AGGAAGAACCCTCAGGAAGGCCTGT |
| | | ACAATGAACTGCAGAAAGATAAGAT |
| | | GGCGGAGGCCTACAGTGAGATTGGG |
| | | ATGAAAGGCGAGCGCCGGAGGGGCA |
| | | AGGGGCACGATGGCCTTTACCAGGG |
| | | TCTCAGTACAGCCACCAAGGACACC |
| | | TACGACGCCCTTCACATGCAGGCCC |
| | | TGCCCCCTCGCGGTAGCGGGGCTAC |
| | | GAACTTCTCCCTTCTTAAACAAGCG |
| | | GGAGACGTGGAAGAAAATCCCGGAC |
| | | CTATGGCCTTACCAGTGACCGCCTT |
| | | GCTCCTGCCGCTGGCCTTGCTGCTC |
| | | CACGCCGCCAGGCCGAACTGGGTGA |
| | | ATGTAATAAGTGATTTGAAAAAAAT |
| | | TGAAGATCTTATTCAATCTATGCAT |
| | | ATTGATGCTACTTTATATACGGAAA |
| | | GTGATGTTCACCCCAGTTGCAAAGT |
| | | AACAGCAATGAAGTGCTTTCTCTTG |
| | | GAGTTACAAGTTATTTCACTTGAGT |
| | | CCGGAGATGCAAGTATTCATGATAC |
| | | AGTAGAAAATCTGATCATCCTAGCA |
| | | AACAACAGTTTGTCTTCTAATGGGA |
| | | ATGTAACAGAATCTGGATGCAAAGA |
| | | ATGTGAGGAACTGGAGGAAAAAAAT |
| | | ATTAAAGAATTTTTGCAGAGTTTTG |
| | | TACATATTGTCCAAATGTTCATCAA |
| | | CACTTCTTGA |
| 57 | anti-CD20 | ATGTCCGTGCCTACCCAGGTGCTGG |
| | CAR + sIL 15 | GCCTGCTGCTGCTGTGGCTGACCGA |
| | nucleotide | CGCCAGATGCGAAATAGTGATGACG |
| | | CAGTCTCCAGCCACCCTGTCTGTGT |
| | | CTCCAGGGGAAAGAACCACCCTCTC |
| | | CTGCAGGGCCAGTCAGAGTGTTAGC |
| | | AGCAACTTAGCCTGGTACCTTCAGA |
| | | AACCTGGCCAGGCTCCCAGGCTCCT |
| | | CATCTATGGTGCATCCACCAGGGCC |
| | | ACTGGTATCCCAGCCAGGTTCAGTG |
| | | GCAGTGGGTCTGGGACAGAGTTCAT |
| | | TCTCACCATCAGCAGCCTGCAGTCT |
| | | GAAGATTTTGCAGTTTATTACTGTC |
| | | AGCAGTATAATAACTGGCCGATCAC |
| | | CTTCGGCCAAGGGACACGGCTGGAG |
| | | ATTAAAGGTGGAGGTGGATCTGGAG |
| | | GAGGAGGATCCGGTGGAGGAGGTGA |
| | | AGTGCAACTGGTGGAGTCTGGGGGA |
| | | GGCTTGGTACAGCCTGGCAGGTCCC |
| | | TGAGACTCTCCTGTGCAGCCTCTGG |
| | | ATTCACCTTTTATGATTATGCCATG |
| | | CACTGGGTCCGGCAAGCTCCAGGGA |
| | | AGGGCCTGGAGTGGGTCTCAGGTAT |
| | | TAGTTGGAATAGTGGTTACATAGGC |
| | | TATGCGGACTCTGTGAAGGGCCGAT |
| | | TCACCATCTCCAGAGACAACGCCAA |
| | | GAACTCCCTGTATCTGCAAATGAAC |
| | | AGTCTGAGAGCTGAGGACACGGCCT |
| | | TGTATTACTGTGCAAAAGATAACAG |
| | | CTATGGAAAGTTCTACTACGGTTTG |
| | | GACGTCTGGGGCCAAGGGACCACGG |
| | | TCACCGTCTCCTCAACCACGACGCC |
| | | AGCGCCGCGACCACCAACACCGGCG |
| | | CCCACCATCGCGTCGCAGCCCCTGT |
| | | CCCTGCGCCCAGAGGCGTGCCGGCC |
| | | AGCGGCGGGGGGCGCAGTGCACACG |
| | | AGGGGGCTGGACTTCGCCTGTGATA |
| | | TCTACATCTGGGCGCCCTTGGCCGG |
| | | GACTTGTGGGGTCCTTCTCCTGTCA |
| | | CTGGTTATCACCCTTTACTGCAAAC |
| | | GGGGCAGAAAGAAACTCCTGTATAT |
| | | ATTCAAACAACCATTTATGAGACCA |
| | | GTACAAACTACTCAAGAGGAAGATG |
| | | GCTGTAGCTGCCGATTTCCAGAAGA |
| | | AGAAGAAGGAGGATGTGAACTGAGA |
| | | GTGAAGTTCAGCAGGAGCGCAGACG |
| | | CCCCCGCGTACCAGCAGGGCCAGAA |
| | | CCAGCTCTATAACGAGCTCAATCTA |
| | | GGACGAAGAGAGGAGTACGATGTTT |
| | | TGGACAAGAGACGTGGCCGGGACCC |
| | | TGAGATGGGGGGAAAGCCGCAGAGA |
| | | AGGAAGAACCCTCAGGAAGGCCTGT |
| | | ACAATGAACTGCAGAAAGATAAGAT |
| | | GGCGGAGGCCTACAGTGAGATTGGG |
| | | ATGAAAGGCGAGCGCCGGAGGGGCA |
| | | AGGGGCACGATGGCCTTTACCAGGG |
| | | TCTCAGTACAGCCACCAAGGACACC |
| | | TACGACGCCCTTCACATGCAGGCCC |
| | | TGCCCCCTCGCGGTAGCGGGGCTAC |
| | | GAACTTCTCCCTTCTTAAACAAGCG |
| | | GGAGACGTGGAAGAAAATCCCGGAC |
| | | CTATGAGAATTTCGAAACCACATTT |
| | | GAGAAGTATTTCCATCCAGTGCTAC |
| | | TTGTGTTTACTTCTAAACAGTCATT |
| | | TTCTAACTGAAGCTGGCATTCATGT |
| | | CTTCATTTTGGGCTGTTTCAGTGCA |
| | | GGGCTTCCTAAAACAGAAGCCAACT |
| | | GGGTGAATGTAATAAGTGATTTGAA |
| | | AAAAATTGAAGATCTTATTCAATCT |
| | | ATGCATATTGATGCTACTTTATATA |
| | | CGGAAAGTGATGTTCACCCCAGTTG |
| | | CAAAGTAACAGCAATGAAGTGCTTT |
| | | CTCTTGGAGTTACAAGTTATTTCAC |
| | | TTGAGTCCGGAGATGCAAGTATTCA |
| | | TGATACAGTAGAAAATCTGATCATC |
| | | CTAGCAAACAACAGTTTGTCTTCTA |
| | | ATGGGAATGTAACAGAATCTGGATG |
| | | CAAAGAATGTGAGGAACTGGAGGAA |
| | | AAAAATATTAAAGAATTTTTGCAGA |
| | | GTTTTGTACATATTGTCCAAATGTT |
| | | CATCAACACTTCTTGA |
| 58 | anti-CD20- | MSVPTQVLGLLLLWLTDARCEIVMT |
| | CAR | QSPATLSVSPGERTTLSCRASQSVS |
| | polypeptide | SNLAWYLQKPGQAPRLLIYGASTRA |
| | | TGIPARFSGSGSGTEFILTISSLQS |
| | | EDFAVYYCQQYNNWPITFGQGTRLE |
| | | IKGGGGSGGGGSGGGGEVQLVESGG |
| | | GLVQPGRSLRLSCAASGFTFYDYAM |
| | | HWVRQAPGKGLEWVSGISWNSGYIG |
| | | YADSVKGRFTISRDNAKNSLYLQMN |
| | | SLRAEDTALYYCAKDNSYGKFYYGL |
| | | DVWGQGTTVTVSSTTTPAPRPPTPA |
| | | PTIASQPLSLRPEACRPAAGGAVHT |
| | | RGLDFACDIYIWAPLAGTCGVLLLS |
| | | LVITLYCKRGRKKLLYIFKQPFMRP |
| | | VQTTQEEDGCSCRFPEEEEGGCELR |
| | | VKFSRSADAPAYQQGQNQLYNELNL |
| | | GRREEYDVLDKRRGRDPEMGGKPQR |
| | | RKNPQEGLYNELQKDKMAEAYSEIG |
| | | MKGERRRGKGHDGLYQGLSTATKDT |
| | | YDALHMQALPPR* |
| 59 | Secretion | MALPVTALLLPLALLLHAARPNWVN |
| | signal | VISDLKKIEDLIQSMHIDATLYTES |
| | and sIL-15 | DVHPSCKVTAMKCFLLELQVISLES |
| | domain | GDASIHDTVENLIILANNSLSSNGN |
| | polypeptide | VTESGCKECEELEEKNIKEFLQSFV |
| | | HIVQMFINTS* |
| 60 | anti-CD20 | ATGTCCGTGCCTACCCAGGTGCTGG |
| | CAR + SIL 15 | GCCTGCTGCTGCTGTGGCTGACCGA |
| | nucleotide | CGCCAGATGCGAAATAGTGATGACG |
| | | CAGTCTCCAGCCACCCTGTCTGTGT |
| | | CTCCAGGGGAAAGAACCACCCTCTC |
| | | CTGCAGGGCCAGTCAGAGTGTTAGC |
| | | AGCAACTTAGCCTGGTACCTTCAGA |
| | | AACCTGGCCAGGCTCCCAGGCTCCT |
| | | CATCTATGGTGCATCCACCAGGGCC |
| | | ACTGGTATCCCAGCCAGGTTCAGTG |
| | | GCAGTGGGTCTGGGACAGAGTTCAT |
| | | TCTCACCATCAGCAGCCTGCAGTCT |
| | | GAAGATTTTGCAGTTTATTACTGTC |
| | | AGCAGTATAATAACTGGCCGATCAC |
| | | CTTCGGCCAAGGGACACGGCTGGAG |
| | | ATTAAAGGTGGAGGTGGATCTGGAG |
| | | GAGGAGGATCCGGTGGAGGAGGTGA |
| | | AGTGCAACTGGTGGAGTCTGGGGGA |
| | | GGCTTGGTACAGCCTGGCAGGTCCC |
| | | TGAGACTCTCCTGTGCAGCCTCTGG |
| | | ATTCACCTTTTATGATTATGCCATG |
| | | CACTGGGTCCGGCAAGCTCCAGGGA |
| | | AGGGCCTGGAGTGGGTCTCAGGTAT |
| | | TAGTTGGAATAGTGGTTACATAGGC |
| | | TATGCGGACTCTGTGAAGGGCCGAT |
| | | TCACCATCTCCAGAGACAACGCCAA |
| | | GAACTCCCTGTATCTGCAAATGAAC |
| | | AGTCTGAGAGCTGAGGACACGGCCT |
| | | TGTATTACTGTGCAAAAGATAACAG |
| | | CTATGGAAAGTTCTACTACGGTTTG |
| | | GACGTCTGGGGCCAAGGGACCACGG |
| | | TCACCGTCTCCTCAACCACGACGCC |
| | | AGCGCCGCGACCACCAACACCGGCG |
| | | CCCACCATCGCGTCGCAGCCCCTGT |
| | | CCCTGCGCCCAGAGGCGTGCCGGCC |
| | | AGCGGCGGGGGGCGCAGTGCACACG |
| | | AGGGGGCTGGACTTCGCCTGTGATA |
| | | TCTACATCTGGGCGCCCTTGGCCGG |
| | | GACTTGTGGGGTCCTTCTCCTGTCA |
| | | CTGGTTATCACCCTTTACTGCAAAC |
| | | GGGGCAGAAAGAAACTCCTGTATAT |
| | | ATTCAAACAACCATTTATGAGACCA |
| | | GTACAAACTACTCAAGAGGAAGATG |
| | | GCTGTAGCTGCCGATTTCCAGAAGA |
| | | AGAAGAAGGAGGATGTGAACTGAGA |
| | | GTGAAGTTCAGCAGGAGCGCAGACG |
| | | CCCCCGCGTACCAGCAGGGCCAGAA |
| | | CCAGCTCTATAACGAGCTCAATCTA |
| | | GGACGAAGAGAGGAGTACGATGTTT |
| | | TGGACAAGAGACGTGGCCGGGACCC |
| | | TGAGATGGGGGGAAAGCCGCAGAGA |
| | | AGGAAGAACCCTCAGGAAGGCCTGT |
| | | ACAATGAACTGCAGAAAGATAAGAT |
| | | GGCGGAGGCCTACAGTGAGATTGGG |
| | | ATGAAAGGCGAGCGCCGGAGGGGCA |
| | | AGGGGCACGATGGCCTTTACCAGGG |
| | | TCTCAGTACAGCCACCAAGGACACC |
| | | TACGACGCCCTTCACATGCAGGCCC |
| | | TGCCCCCTCGCTAGAGTACTGCGGC |
| | | CGCTACGTAAATTCCGCCCCTCTCC |
| | | CTCCCCCCCCCCTAACGTTACTGGC |
| | | CGAAGCCGCTTGGAATAAGGCCGGT |
| | | GTGCGTTTGTCTATATGTTATTTTC |
| | | CACCATATTGCCGTCTTTTGGCAAT |
| | | GTGAGGGCCCGGAAACCTGGCCCTG |
| | | TCTTCTTGACGAGCATTCCTAGGGG |
| | | TCTTTCCCCTCTCGCCAAAGGAATG |
| | | CAAGGTCTGTTGAATGTCGTGAAGG |
| | | AAGCAGTTCCTCTGGAAGCTTCTTG |
| | | AAGACAAACAACGTCTGTAGCGACC |
| | | CTTTGCAGGCAGCGGAACCCCCCAC |
| | | CTGGCGACAGGTGCCTCTGCGGCCA |
| | | AAAGCCACGTGTATAAGATACACCT |
| | | GCAAAGGCGGCACAACCCCAGTGCC |
| | | ACGTTGTGAGTTGGATAGTTGTGGA |
| | | AAGAGTCAAATGGCTCTCCTCAAGC |
| | | GTATTCAACAAGGGGCTGAAGGATG |
| | | CCCAGAAGGTACCCCATTGTATGGG |
| | | ATCTGATCTGGGGCCTCGGTGCACA |
| | | TGCTTTACATGTGTTTAGTCGAGGT |
| | | TAAAAAAACGTCTAGGCCCCCCGAA |
| | | CCACGGGGACGTGGTTTTCCTTTGA |
| | | AAAACACGATGATATTAATTAAGCC |
| | | ACCGCCATGGCCTTACCAGTGACCG |
| | | CCTTGCTCCTGCCGCTGGCCTTGCT |
| | | GCTCCACGCCGCCAGGCCGAACTGG |
| | | GTGAATGTAATAAGTGATTTGAAAA |
| | | AAATTGAAGATCTTATTCAATCTAT |
| | | GCATATTGATGCTACTTTATATACG |
| | | GAAAGTGATGTTCACCCCAGTTGCA |
| | | AAGTAACAGCAATGAAGTGCTTTCT |
| | | CTTGGAGTTACAAGTTATTTCACTT |
| | | GAGTCCGGAGATGCAAGTATTCATG |
| | | ATACAGTAGAAAATCTGATCATCCT |
| | | AGCAAACAACAGTTTGTCTTCTAAT |
| | | GGGAATGTAACAGAATCTGGATGCA |
| | | AAGAATGTGAGGAACTGGAGGAAAA |
| | | AAATATTAAAGAATTTTTGCAGAGT |
| | | TTTGTACATATTGTCCAAATGTTCA |
| | | TCAACACTTCTTGA |

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

What is claimed is:

1. A method of treating a relapsed/refractory (R/R) B cell malignancy in a patient in need thereof, the method comprising administering to the subject a therapeutically effective amount of anti-CD20 CAR γδ T cells that express a chimeric antigen receptor (CAR) comprising a binding domain that specifically binds to CD20 on a malignant B cell, thereby treating the patient;
wherein the patient has been previously treated with at least one, at least two, or at least three, previous therapies.

2. The method of claim 1, wherein the patient is relapsed from and/or refractory to a previous therapy comprising an anti-CD20 monoclonal antibody; optionally wherein the anti-CD20 monoclonal antibody is rituximab.

3. The method of claim 1, wherein the patient is relapsed from and/or refractory to a previous therapy comprising an anti-CD19 CAR αβ T cell therapy; optionally wherein the anti-CD19 CAR αβ T cell therapy is selected from axicabtagene ciloleucel and tisagenlecleucel.

4. The method of claim 2, wherein the binding domain specifically binds to a CD20 epitope that is different from the CD20 epitope recognized by the anti-CD20 monoclonal antibody.

5. The method of any one of claims 1-4, wherein the therapeutically effective amount of said anti-CD20 CAR γδ T cells is between about 3×10⁷ and about 1×10⁹ γδ T cells.

6. The method of any one of claims 1-4, wherein the therapeutically effective amount of said anti-CD20 CAR γδ T cells is about 3×10⁷ γδ T cells.

7. The method of any one of claims 1-4, wherein the therapeutically effective amount of said anti-CD20 CAR γδ T cells is about 1×10⁸ γδ T cells.

8. The method of any one of claims 1-4, wherein the therapeutically effective amount of said anti-CD20 CAR γδ T cells is about 3×10⁸ γδ T cells.

9. The method of any one of claims 1-4, wherein the therapeutically effective amount of said anti-CD20 CAR γδ T cells is about 1×10⁹ γδ T cells.

10. The method of any preceding claim, further comprising administering to the subject a lymphodepletion (LD) regimen prior to administering to the subject a first dose of the therapeutically effective amount of anti-CD20 CAR γδ T cells.

11. The method of claim 10, wherein the LD regimen comprises administration of fludarabine at about 30 mg/m²/day plus cyclophosphamide at about 500 mg/m²/day for three days, optionally further comprising an anti-CD52 antibody and/or an anti-CD19 antibody.

12. The method of claim 10, wherein the LD regimen comprises administration of fludarabine at about 30 mg/m²/day for four days, plus cyclophosphamide at about 1000 mg/m²/day for three days, optionally further comprising an anti-CD52 antibody and/or an anti-CD19 antibody.

13. The method of any one of claims 1-12, the method further comprising administering one or more additional doses of anti-CD20 CAR γδ T cells at least 5 days, at least 7 days, at least 10 days, at least 14 days, at least 21 days, at least 28 days, or at least one month after the first dose.

14. The method of claim 13, wherein the one or more additional doses of anti-CD20 CAR γδ T cells are administered without an additional LD regimen.

15. The method of claim 13, wherein the one or more additional doses of anti-CD20 CAR γδ T cells are administered following an additional LD regimen.

16. The method of any one of claims 13-15, wherein the one or more additional doses comprise an increased amount of anti-CD20 CAR γδ T cells, a decreased amount of anti-CD20 CAR γδ T cells, or the same amount of anti-CD20 CAR γδ T cells.

17. The method of any one of claims 13-16, wherein the one or more additional doses comprise anti-CD20 CAR γδ T cells derived from the same donor.

18. The method of any one of claims 13-16, wherein the one or more additional doses comprise anti-CD20 CAR γδ T cells derived from a different donor.

19. The method of any preceding claim, wherein the anti-CD20 CAR γδ T cells are γδ1 T cells, γδ2 T cells, γδ3 T cells, or γβ4 T cells, preferably wherein the γδ T cells are γδ1 T cells.

20. The method of any one of claims 1-19, further comprising:
monitoring the subject for one or more pharmacodynamics/pharmacokinetics biomarkers following administration of the therapeutically effective amount of anti-CD20 CAR γδ T cells, wherein the biomarkers are selected from the group comprising or consisting of CAR transgene expression level, quantitative measurement of CAR+γδ T cells, serum level of one or more cytokines and/or serum proteins, and minimal residual disease (MRD).

21. The method of claim 20, wherein the one or more cytokines/serum proteins are selected from the group comprising or consisting of INFγ, GM-CSF, IL-2, IL-7, IL-15, TNFα, IL-1β, IL-6, IL-8, IL-10, MIP1α, MIP1β, CRP, ferritin, monocyte chemotactic protein-1 (MCP-1), CXCL9, CXCL10, CXCL11, CCL5, IL-5, IL-IRA, IL-18, soluble MICA, IL-10, IL-4, IL-13, IL-17, CCL2, CXCL12, CCL17, and CCL22.

22. The method of claim 21, wherein the one or more cytokines are IL-2 and/or IL-8.

23. The method of claim 20, further comprising measuring CAR transgene expression level via quantitative polymerase chain reaction (qPCR).

24. The method of claim 20, wherein the quantitative measurement of CAR+γδ T cells is determined via flow cytometry.

25. The method of claim 20, further comprising conducting analysis of MRD via immunosequencing methodology.

26. The method of any one of claims 20-25, further comprising administering a secondary treatment regimen based at least in part on monitoring one or more of said biomarkers.

27. The method of claim 26, wherein the secondary treatment regimen comprises one or more additional doses of anti-CD20 CAR γδ T cells.

28. The method of claim 27, wherein the one or more additional doses of anti-CD20 CAR γδ T cells are administered without an additional LD regimen.

29. The method of claim 27, wherein the one or more additional doses of anti-CD20 CAR γδ T cells are administered following an additional LD regimen.

30. The method of any one of claims 27-29, wherein the one or more additional doses comprise an increased amount of anti-CD20 CAR γδ T cells a decreased amount of anti-CD20 CAR γδ T cells, or the same amount of anti-CD20 CAR γδ T cells.

31. The method of any one of claims 27-29, wherein the one or more additional doses comprise anti-CD20 CAR γδ T cells derived from a different donor.

32. The method of any one of claims 26-31, wherein the follow-on treatment regimen further comprises administration of cyclophosphamide, doxorubicin hydrochloride (hydroxydaunorubicin), vincristine sulfate, and prednisone (CHOP).

33. The method of any one of claims 1-32, wherein said B cell malignancy is selected from the group comprising or consisting of diffuse large B cell lymphoma (DLBCL), primary mediastinal large B cell lymphoma (PMBCL), mantle cell lymphoma (MCL), and transformed follicular lymphoma (tFL).

34. A method of treating a relapsed/refractory (R/R) B cell malignancy in a patient in need thereof, the method comprising administering to the subject a lymphodepletion (LD) regimen at least five days prior to administering to the subject a first dose of anti-CD20 CAR γδ T cells, followed by administering to the subject the first dose comprising a therapeutically effective amount of anti-CD20 CAR γδ T cells that express a chimeric antigen receptor (CAR) comprising a binding domain that specifically binds to CD20 on a malignant B cell, thereby treating the patient; wherein the patient has been previously treated with at least one, at least two, or at least three, previous therapies.

35. The method of claim 34, further comprising administering a second dose comprising a therapeutically effective dose of anti-CD20 CAR γδ T cells to the subject at least 5 days, at least 7 days, at least 10 days, at least 14 days, at least 21 days, at least 28 days, or at least one month after the first dose, with or without administering an additional LD regimen.

36. The method of claim 35, wherein the second therapeutically effective dose comprises an increased amount of anti-CD20 CAR γδ T cells, a decreased amount of anti-CD20 CAR γδ T cells, or the same amount of anti-CD20 CAR γδ T cells as the first dose.

37. The method of claim 36, wherein the second dose is administered seven days after the first dose, the therapeutically effective amount of said anti-CD20 CAR γδ T cells in the first and second doses is about 3×10⁸ γδ T cells, and the second dose is administered without administering an additional LD regimen.

38. The method of any one of claims 35-37, further comprising administering a third dose comprising a therapeutically effective amount of anti-CD20 CAR γδ T cells to the subject at least 5 days, at least 7 days, at least 10 days, at least 14 days, at least 21 days, at least 28 days, or at least one month after the second dose, with or without administering an additional LD regimen.

39. The method of claim 38, wherein the third therapeutically effective dose comprises an increased amount of anti-CD20 CAR γδ T cells, a decreased amount of anti-CD20 CAR γδ T cells, or the same amount of anti-CD20 CAR γδ T cells as the first and/or second doses.

40. The method of claim 39, wherein the third dose is administered seven days after the second dose, the therapeutically effective amount of said anti-CD20 CAR γδ T cells in the first, second and third doses is about 3×10⁸ γδ T cells, and the second and third doses are administered without administering an additional LD regimen.

41. The method of any of the preceding claims, wherein the CAR comprises the amino acid sequence of SEQ ID NO: 41.

42. The method of 41, wherein the anti-CD20 γδ T cells further comprise an isolated nucleic acid encoding the CAR.

43. The method of any of claims 1-41, wherein the anti-CD20 CAR γδ T cells further comprise an isolated nucleic acid encoding the CAR having the sequence of SEQ ID NO: 46.
